(19) United States
(12) Patent Application Publication
(10) Pub. No.: US 2008/0280974 A1
(43) Pub. Date: Nov. 13, 2008

(54) Spiro compounds for treatment of inflammatory disorders

(76) Inventors: M. David Weingarten, Cumming, GA (US); Liming Ni, Duluth, GA (US); Zhihong Ye, Lilburn, GA (US); Charles Q. Meng, Duluth, GA (US); Raymond Ng, Alpharetta, GA (US); Jim A. Sikorski, Atlanta, GA (US)

Correspondence Address: KING & SPALDING LLP, 1180 PEACHTREE STREET, ATLANTA, GA, 30309-3521, US

(21) Appl. No.: 12/151,919

(22) Filed: May 9, 2008

Related U.S. Application Data
(60) Provisional application No. 60/928,477, filed on May 9, 2007.

(51) Int. Cl. A61K 31/352; C07D 493/10; A61P 29/00; A61P 11/00

(52) U.S. Cl. 514/453; 549/344

(57) ABSTRACT

Provided are compounds, pharmaceutical compositions and methods of treatment or prophylaxis of an inflammatory condition, in particular asthma. The compounds are of the general Formula I, or a pharmaceutically acceptable salt, ester, prodrug or derivative thereof:

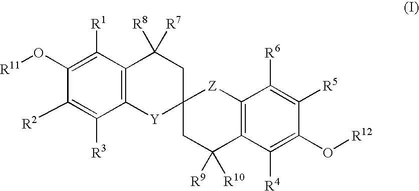

wherein Y, Z and R¹-R¹² are defined herein.

### CROSS-REFERENCE TO RELATED APPLICATIONS

This application claims priority to U.S. provisional application No. 60/928,477, filed May 9, 2007, the disclosure of which is hereby incorporated by reference.

### FIELD OF THE INVENTION

The present invention is in the area of methods and compositions for the treatment and prophylaxis of inflammatory disorders and, in particular, for the treatment or prophylaxis of respiratory inflammatory diseases such as asthma.

### BACKGROUND OF THE INVENTION

A growing consensus among scientists is that common disorders such as asthma, atherosclerosis, colon cancer, and Alzheimer's disease are all caused in part by a chronic inflammatory syndrome. Generally, chronic inflammation is involved in diseases as diverse as allergy, anemia, aortic valve stenosis, arthritis, atherosclerosis, cancer, heart valve dysfunction, obesity, diabetes, congestive heart failure, digestive system diseases, and Alzheimer's disease (Brouqui et al. 1994; Devaux et al. 1997; De Keyser et al. 1998). Chronic inflammation inevitably causes tissue damage and is accompanied by simultaneous attempts at healing and repair. The exact nature, extent and time course of chronic inflammation is variable, and depends on a balance between the causative agent and the attempts of the body to remove it. Disorders associated with inflammation are debilitating to individuals suffering from them and cost billions in reduced productivity and increased medical expenses.

Asthma is one of the most common chronic health conditions and is on the rise due to irritants such as pollution and chronic exposure to indoor allergens such as cigarette smoke, cockroaches, dust mites, mold, animals, pollen, cold air, exercise, stress, and respiratory infections. Asthma and related respiratory disorders such as chronic obstructive pulmonary disease (COPD) are chronic or recurring inflammatory conditions in which the airway develops increased responsiveness to various stimuli, characterized by bronchial hyper-responsiveness, inflammation, increased mucus production, and intermittent airway obstruction.

Coronary heart disease (CHD) remains the leading cause of death in the industrialized countries. Cardiovascular disease has been linked to several causative factors, which include hypercholesterolemia, hyperlipidemia, and the expression of VCAM-1 in vascular endothelial cells. The primary cause of CHD is atherosclerosis, a disease characterized by the deposition of lipids in the arterial vessel wall, resulting in a narrowing of the vessel passages and ultimately hardening the vascular system, which is likely mediated by the expression of certain inflammatory cytokines and VCAM-1. Atherosclerosis as manifested in its major clinical complication, ischemic heart disease, continues to be a major cause of death in industrialized countries. It is now well accepted that atherosclerosis can begin with local injury to the arterial endothelium followed by proliferation of arterial smooth muscle cells from the medial layer to the intimal layer along the deposition of lipid and accumulation of foam cells in the lesion. As the atherosclerotic plaque develops it progressively occludes more and more of the affected blood vessel and can eventually lead to ischaemia or infarction.

Cells that are chronically exposed to higher than normal levels of polyunsaturated fatty acids or their oxidized counterparts can initiate an immune response that is not normal and which is out of proportion to the threat presented, leading to a diseased state. The oversensitization of vascular endothelial cells to PUFAs and ox-PUFAs can accelerate the formation, for example, of atherosclerotic plaque.

Many inflammatory disorders are mediated by certain cytokines. These include the IL-6 and IL-8 families. Regulation of these and other related cytokines can be a strategy when overstimmulation of the immune responses leads to adverse events.

Cytokines are produced predominantly by activated immune cells such as microglia and are involved in the amplification of inflammatory reactions. These include IL-1, IL-6, TNF-α, and TGF-β.

### Spiro Compounds

A spiro compound is a bicyclic organic compound with rings connected through just one atom. The rings can be different in nature or identical. The connecting atom is also called the spiroatom, most often a quaternary carbon (“spiro carbon”).

Some spiro compounds exhibit axial chirality. Spiroatoms can be centers of chirality even when they lack the four different substituents normally observed in chirality. When two rings are identical, the priority is determined by a slight modification of the CIP system assigning a higher priority to one ring extension and a lower priority to an extension in the other ring. When rings are dissimilar the regular rules apply.

Spiro forms of lactones and oxazines are frequently used as leuco dyes, frequently displaying chromism—reversible change between their colorless and color form.

Spiroketals, or spiroacetals, are substructures in many naturally occurring substances, including insects, microbes, plants, fingi, and marine organisms (F. Perron and K. F. Albizati, Chem. Rev. 1989, 89, pp. 1617-1661). Most naturally occurring spiroketal compounds include one of the substructures (A) 1,7-Dioxaspiro[5.5]undecane, (B) 1,6-Dioxaspiro[4.5]decane, or (C) 1,7-Dioxaspiro[4.4]nonane.

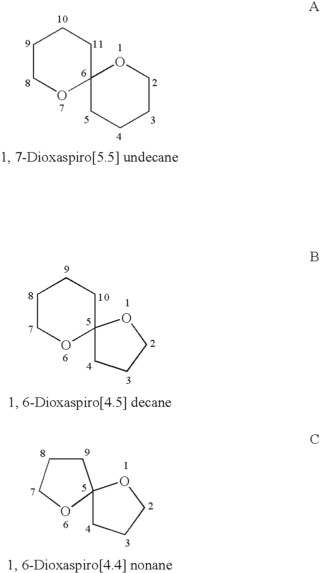

Reveromycin A is a naturally occurring spiroketal compound isolated from the soil actinomycete genus Streptomyces sp. Reveromycin A is known to inhibits bone resorption in vitro and in vivo by inducing apoptosis specifically in ostesoclasts, suggesting that RM-A might be useful in the treatment of bone disorders, including osteoporosis. (J.-T. Woo, et al. PNAS, Mar. 21, 2006; 103(12): 4729-4734.) Reveromycin A, is also known to have antifungal and antiproliferative properties. (Takahashi, H. et al. (1992) J. Antibiot. 45, 1414-1419; Miyamoto, Y. et al., J. Biol. Chem., Vol. 277, Issue 32, 28810-28814, Aug. 9, 2002.) The structure of reveromycin A includes a [6,6] spiroketal core. The enantioselective sysnthesis of the spiroketal core of reveromycin A has recently been achieved. (K. Drouet, et al., Org. Lett. 2000, Vol. 2., No. 2, pp. 207-210).

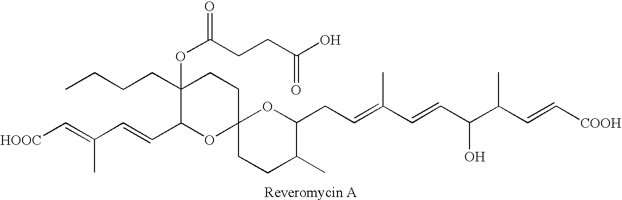

Spongistatin is a potent tubulin depolymerizing natural product isolated from an Eastern Indian Ocean sponge in the genus Spongia. (Bai et al. Mol. Pharmacol. 1993; 44: 757-766).

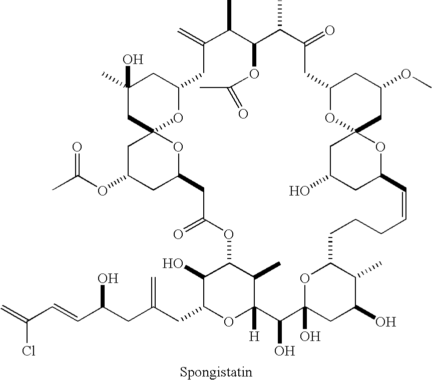

U.S. Pat. No. 6,335,364 to Parker Hughes Institute describes spiroketal pyrane compounds as effective agents for inhibiting cellular proliferation, and as effective anti-cancer agents. The '364 patent describes spiroketal pyrane compounds including compounds of the formula:

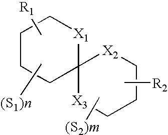

wherein X₁, X₂, and X₃ are the same or different, and are each independently O, C, or S;

R₁ and R₂ are the same or different and are each independently H, provided both R₁ and R₂ are not H, or (C1-C8)alkyl, (C1-C8)cycloalkyl, (C1-C8)alkoxy, (C1-C8)aryloxy, (C1-C8)arylthio, (C1-C8)aryl, (C1-C8)heteroaryl, C═NRaRb or NRaRb; wherein Ra and Rb are each independently hydrogen, acyl, (C1-C8)alkyl, (C3-C7)cycloalkyl, (C6-C10)aryl, or (C6-C10)heteroaryl, or Ra and Rb together with the nitrogen to which they are attached form a ring such as pyrrolidino, piperidino, morpholino, or thiomorpholino; n and m are the same or different, and are each independently 0 to 7; S₁ and S₂ can be the same or different, and are each independently OH, SH, CO₂H, halogen, CN, acyl, thioacyl, ester, thioester, (C1-C6)alkoxy, (C1-C6)aryloxy, (C1-C6)alkylthio, (C1-C6)arylthio, (C1-C6)alkyl, (C1-C6)alkenyl, (C1-C6)alkynyl, (C3-C7)cycloalkyl, (C6-C10)aryl, or (C6-C10)heteroaryl, C(O)NRaRb or NRaRb; wherein Ra and Rb are each independently hydrogen, acyl, (C1-C6)alkyl, (C3-C7)cycloalkyl, (C6-C10)aryl, or (C6-C10)heteroaryl, or Ra and Rb together with the nitrogen to which they are attached form a ring such as pyrrolidino, piperidino, morpholino, or thiomorpholino; and taken together, any two S1 and S2 can form a ring, and any two adjacent substituents can form a double bond between the two carbons to which they are attached. The hydrocarbon moieties of R₁, R₂, S₁, and S₂ may be substituted or unsubstituted.

U.S. Pat. Nos. 4,952,470 and 5,278,014 to Konica Corporation describe spirobichroman compounds which can be used in an electrophotographic photosensitive member to inhibit ozone-induced oxidation. For example, the '014 patent includes the spirobichroman compounds of the formula:

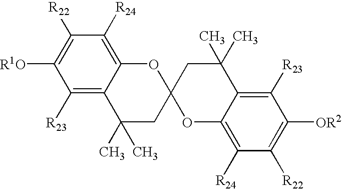

wherein R₂₂ represents an alkyl, alkenyl, aryl, alkoxy, alkenoxy, or aryloxy group; R₂₃ and R₂₄ each represent a hydrogen, halogen, alkyl, alkenyl, or alkoxy group; R¹ represents an alkyl, alkenyl, cycloalkyl, aryl, heterocyclic, R₂₅CO—, R₂₆SO₂—, or R₂₇NHCO— group; R² represents a hydrogen, alkyl, alkenyl, R₂₅CO—, R₂₆SO₂— or R₂₇NHCO— group; R₂₅, R₂₆ and R₂₇ each represent an alkyl, alkenyl, cycloalkyl, aryl, or heterocyclic group.

U.S. Pat. Nos. 4,174,220 and 4,159,910 to Konishiroku Photo Industry Co., Ltd. describes silver halide color photographic materials which may incorporate anti-discoloration agents of the formula:

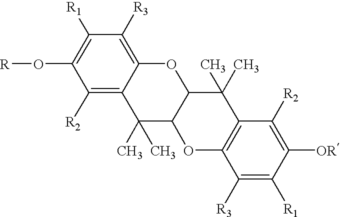

wherein R₁ represents an alkyl, alkenyl, alkoxyl, alkenoxyl or aryloxyl group; R₂ and R₃ individually represent hydrogen, halogen, alkyl, alkenyl or alkoxyl group; R represents an alkyl, alkenyl, cycloalkyl, aryl, heterocyclic, R₆CO—, R₇SO₂— or R₈NHCO—; R′ is hydrogen, R₆CO—, R₇SO₂— or R₈NHCO—; R₆, R₇ and R₈ individually represent an alkyl, alkenyl, cycloalkyl, aryl or heterocyclic; and, the respective above-mentioned groups may be substituted by halogen, alkyl, aryl, aryloxyl, cyano, acyloxyl, carboalkoxyl, acyl, sulfamoyl, hydroxyl, nitro or amino group. When R′ represents R₆CO—, R₇SO₂— or R₈NHCO—, R may be either the same with R′ or different from R′.

U.S. Pat. No. 4,713,317 to Konishiroku Photo Industry Co., Ltd. describes silver halide color photographic materials which may incorporate anti-discoloration agents of the formula:

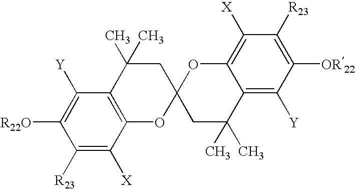

wherein R₂₂ and R_{′22} which may be the same or different and are each hydrogen, alkyl, alkenyl, cycloalkyl, aryl, a heterocyclic, —CO—V′, —SO2-V′ or —CONH—V′ wherein V′ is an alkyl of 1-20 carbons, alkoxy of 1-20 carbons, aryloxy, alkyloxycarbonyl, or aryloxycarbonyl; R₂₃ is an alkyl of 1-20 carbons, alkenyl of 2-20 carbons, aryl, alkoxy of 1-20 carbons, or aryloxy; when both R₂₂ or R_{′22} and R₂₃ are alkyl, R₂₂ or R_{′22} and R₂₃ may be fused to form a 5- to 7-membered ring; X and Y each represent a hydrogen, halogen, alkyl of 1-20 carbons, alkoxy of 1-20 carbons, alkenyl, aryl or aryloxy.

U.S. Pat. No. 4,396,698 to Fuji Photo Film Co., Ltd. describes polymer latex composition comprising hydrophobic substances of the formula

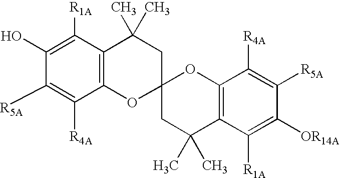

wherein R_{1A}, R_{4A} and R_{5A}, which may be the same or different, each represents a hydrogen, alkyl, aryl, alkenyl, aralkyl, alkoxy, aryloxy, alkenoxy, aralkoxy, alkylthio, arylthio, halogen, hydroxy, amino, acylamino, diacylamino, sulfonamide, alkylamino, dialkylamino, arylamino, heterocyclic amino, sulfo, arylsulfonyl, arylsulfinyl, acyloxy, acyl, alkoxycarbonyl, and R_{14A} represents a hydrogen, alkyl, aryl, aralkyl, alkenyl, acyl, or sulfonyl group.

PCT Publication WO 02/098363 to Agouron Pharmaceuticals, Inc. describes non-peptide furanyl GnRH agents of formula I

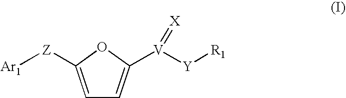

wherein Ar₁ is (un)substituted fused of spiro polycyclic cycloalkyl, heterocycloalkyl, aryl or heteroaryl group; R₁ is (un)substituted aryl, cycloalkyl, heterocycloalkyl, alkyl, alkenyl, etc.; Z is O, S, SO₂, or NR₂; V is SO, S, or C; X is O, N, or S; Y is O, or NR₂; R₂ is H, alkyl or alkoxy. These compounds are described as useful for inhibiting the effect of gonadotropin-releasing hormone and suitable for treating mammalian reproductive disorders and steroid hormone-dependent tumors, and for regulating fertility, where suppression of gonadotropin release is indicated.

There remains a need for improved compounds and methods for the treatment of chronic inflammatory disorders. In particular, there is a need for improved treatments for chronic respiratory inflammatory disorders such as asthma.

It is therefore an object of the present invention to provide new compounds, pharmaceutical compositions and methods for the treatment of inflammatory disorders.

It is a further object of the invention to provide compounds, compositions and methods of treating disorders and diseases mediated by inflammatory cytokines, including respiratory and cardiovascular inflammatory diseases.

### SUMMARY OF THE INVENTION

It has been discovered that certain spiroketal compounds are useful in the treatment or prophylaxis of inflammatory conditions. In particular, compounds described below are useful for treating respiratory inflammation such as found in asthma as well as other inflammatory disorders such as atherosclerosis or arthritis.

In one embodiment, compounds, pharmaceutical compositions and methods of treatment or prophylaxis of an inflammatory condition, and in particular asthma, comprising administering to a host in need thereof a compound of Formula I, or a pharmaceutically acceptable salt, ester, prodrug or derivative thereof are provided:

In one embodiment, pharmaceutical compositions and methods of treatment or prophylaxis of an inflammatory condition, and in particular asthma, comprising administering to a host in need thereof a compound of Formula I, or a pharmaceutically acceptable salt, ester, prodrug or derivative thereof are provided:

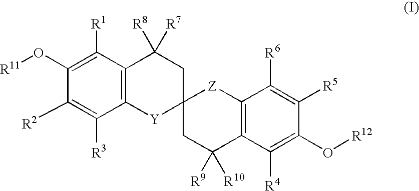

wherein:

Y and Z are independently O, S(O)_{q}, Se(O)_{q} or N(R¹³);

each q is independently 0, 1 or 2;

R¹, R², R³, R⁴, R⁵ and R⁶ are independently selected from the group consisting of hydrogen, halo, C₁-C₆ straight alkyl, C₁-C₆ branched alkyl, C₂-C₆ alkenyl, C₃-C₈ cyclic alkyl, aryl and —OR¹⁴, wherein all may be optionally substituted by a hydroxy group;

R⁷, R⁸, R⁹ and R¹⁰ are independently selected from the group consisting of hydrogen, halo, C₁-C₆ straight alkyl, C₁-C₆ branched alkyl, and C₃-C₈ cyclic alkyl, wherein all may be optionally substituted by a hydroxy group;

R¹¹ and R¹² are independently selected from the group consisting of hydrogen, C₁-C₆ straight alkyl, C₁-C₆ branched alkyl, C₂-C₆ alkenyl, C₃-C₈ cyclic alkyl, and acyl, wherein all may be optionally substituted by one or more independently selected from the group consisting of halo, alkyl, lower alkyl, alkenyl, cycloalkyl, acyl, hydroxy, hydroxyalkyl, carboxy, alkoxy, heterocyclic, heteroaryl, aryl, amino, aminoalkyl, —NR¹⁴R¹⁵, oxo, cyano, alkoxycarbonyl, —OR¹⁶, —C(O)R¹⁶, —C(O)—NH₂, —C(O)—N(H)R⁴, —C(O)—N(H)OR¹⁴, —C(O)—NR¹⁴R¹⁵, —NR¹⁵CO)R¹⁴, —NR¹⁵C(O)NR¹⁴R¹⁵, —OC(O)NR¹⁴R¹⁵, —NR¹⁵C(O)OR¹⁶, —S(O)ₙ—R¹⁶, —S(O)₂—NH₂, —S(O)₂—N(H)R¹⁴ and —S(O)₂—NR¹⁴R¹⁵;

Or R¹¹ and R¹² are independently selected from the group consisting of hydrogen, aryl, heteroaryl and heterocycle, wherein all may be optionally substituted by one or more independently selected from the group consisting of halo, alkyl, lower alkyl, alkenyl, cycloalkyl, acyl, hydroxy, hydroxyalkyl, alkoxy, heterocyclic, heteroaryl, aryl, amino, aminoalkyl, —NR¹⁴R¹⁵, oxo, cyano, carboxy, carboxyalkyl, alkoxycarbonyl, —OR¹⁶, —C(O)R¹⁶, —C(O)—NH₂, —C(O)—N(H)R¹⁴, —C(O)—N(H)OR¹⁴, —C(O)—NR¹⁴R¹⁵, —NR¹⁵C(O)R¹⁴, —NR¹⁵C(O)NR¹⁴R¹⁵, —OC(O)NR¹⁴R¹⁵, —NR¹⁵C(O)OR¹⁶, —S(O)—R¹⁶, —S(O)₂—NH₂, —S(O)₂—N(H)R¹⁴ and —S(O)₂—NR¹⁴R¹⁵;

each n is independently 0, 1, or 2;

R¹³ is independently selected from the group consisting of hydrogen, hydroxy, C₁-C₆ straight alkyl, C₁-C₆ branched alkyl, C₂-C₆ alkenyl, C₃-C₈ cyclic alkyl, heterocyclic, heteroaryl and aryl, wherein all may be optionally substituted by one or more independently selected from the group consisting of halo, alkyl, lower alkyl, acyl, oxo, hydroxy, hydroxyalkyl, alkoxy, heterocyclic, heteroaryl, cyano, amino, aminoalkyl, and carboxy;

R¹⁴ and R¹⁵ are independently selected from the group consisting of hydrogen, C₁-C₆ straight alkyl, C₁-C₆ branched alkyl, C₂-C₆ alkenyl, C₃-C₈ cyclic alkyl, aryl, heteroaryl, heterocycle, and acyl, wherein all may be optionally substituted by one or more independently selected from the group consisting of halo, alkyl, lower alkyl, alkenyl, cycloalkyl, acyl, hydroxy, hydroxyalkyl, alkoxy heterocyclic, heteroaryl, aryl, amino, aminoalkyl, oxo, cyano, carboxy, carboxyalkyl, alkoxycarbonyl, and —OR¹⁶;

R¹⁴ and R¹⁵ taken together may form a 4- to 12-membered monocyclic, bicyclic, tricyclic or benzofused ring;

R¹⁶ is independently selected from the group consisting of C₁-C₆ straight alkyl, C₁-C₆ branched alkyl, C₂-C₆ alkenyl, C₃-C₈ cyclic alkyl, heterocyclic, heteroaryl and aryl, wherein all may be substituted by one or more independently selected from the group consisting of halo, alkyl, lower alkyl, acyl, oxo, hydroxy, hydroxyalkyl, heterocyclic, heteroaryl, aryl, amino, aminoalkyl, cyano, and carboxy;

with the proviso, that when R¹¹ and R¹² are heteroaryl, R¹¹ and R¹² cannot be 2-furyl.

In one embodiment, the pharmaceutical compositions and methods of treatment or prophylaxis of an inflammatory condition, and in particular asthma, comprises administering to a host in need thereof a compound of Formula I, or a pharmaceutically acceptable salt, ester, prodrug or derivative thereof wherein R¹¹ is hydrogen.

In a separate embodiment, compounds, pharmaceutical compositions and methods of treatment or prophylaxis of an inflammatory condition, and in particular asthma, comprising administering to a host in need thereof a compound of Formula II, or a pharmaceutically acceptable salt, ester, prodrug or derivative thereof are provided:

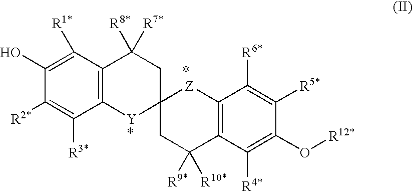

wherein:

Y* and Z* are independently O, S(O)_{q}, Se(O)_{q} or N(R^{13*});

each q is independently 0, 1 or 2;

R^{1*}, R^{2*}, R^{3*}, R^{4*}, R^{15*} and R^{16*} are independently selected from the group consisting of hydrogen, halo, C₁-C₆ straight alkyl, C₁-C₆ branched alkyl, C₂-C₆ alkenyl, C₃-C₈ cyclic alkyl, aryl and —OR¹⁴, wherein all may be optionally substituted by a hydroxy group;

R^{7*}, R^{8*}, R^{9*} and R^{10*} are independently selected from the group consisting of hydrogen, halo, C₁-C₆ straight alkyl, C₁-C₆ branched alkyl, and C₃-C₈ cyclic alkyl, wherein all may be optionally substituted by a hydroxy group;

R^{12*} is selected from the group consisting of C₁-C₆ straight alkyl, hydroxy-C₁-C₆ straight alkyl, polyhydroxy-C₁-C₆ straight alkyl, carboxy-C₁-C₆ straight alkyl, carboxy-C₃-C₆ branched alkyl, carboxy-C₂-C₆ alkenyl, carboxy-C₃-C₈ cyclic alkyl, (C(O)NHR^{13*})—C₁-C₆ straight alkyl, (C(O)NHR^{13*})—C₃-C₆ branched alkyl, (C(O)NHR^{13*})—C₂-C₆ alkenyl, (C(O)NHR^{13*})—C₃-C₈ cyclic alkyl, heteroaralkyl, heterocyclicalkyl, and aralkyl, wherein all may be optionally substituted by one or more independently selected from the group consisting of halo, alkyl, lower alkyl, alkenyl, cycloalkyl, acyl, hydroxy, hydroxyalkyl, heterocyclic, heteroaryl, aryl, amino, aminoalkyl, —NR^{14*}R^{15*}, oxo, cyano, carboxy, carboxyalkyl, alkoxycarbonyl, —OR^{16*}, —C(O)R^{16*}, —C(O)—NH₂, —C(O)—N(H)R^{14*}, —C(O)—N(H)OR^{14*}, C(O)—NR^{14*}R^{15*}, —NR^{15*}C(O)R^{14*}, —NR^{15*}, —OC(O)NR^{14*}R^{15*}, —OC(O)NR^{14*}R^{15*}, —NR^{15*}C(O)OR^{16*}, —S(O)ₙ—R^{16*}, —S(O)₂—NH₂, —S(O)₂—N(H)R^{14*} and —S(O)₂—NR^{14*}R^{15*};

each n is independently 0, 1, or 2;

R^{13*} is selected from the group consisting of hydrogen, hydroxy, C₁-C₆ straight alkyl, C₁-C₆ branched alkyl, C₂-C₆ alkenyl, C₃-C₈ cyclic alkyl, heterocyclic, heteroaryl and aryl, wherein all may be optionally substituted by one or more independently selected from the group consisting of halo, alkyl, lower alkyl, acyl, oxo, hydroxy, hydroxyalkyl, heterocyclic, heteroaryl, cyano, amino, aminoalkyl, and carboxy;

R^{14*} and R^{15*} are independently selected from the group consisting of hydrogen, C₁-C₆ straight alkyl, C₁-C₆ branched alkyl, C₂-C₆ alkenyl, C₃-C₈ cyclic alkyl, aryl, heteroaryl, heterocycle, and acyl, wherein all may be optionally substituted by one or more independently selected from the group consisting of halo, alkyl, lower alkyl, alkenyl, cycloalkyl, acyl, hydroxy, hydroxyalkyl, heterocyclic, heteroaryl, aryl, amino, aminoalkyl, oxo, cyano, carboxy, carboxyalkyl, alkoxycarbonyl, and —OR^{16*};

R^{14*} and R^{15*} taken together may form a 4- to 12-membered monocyclic, bicyclic, tricyclic or benzofused ring;

R^{16*} is independently selected from the group consisting of C₁-C₆ straight alkyl, C₁-C₆ branched alkyl, C₂-C₆ alkenyl, C₃-C₈ cyclic alkyl, heterocyclic, heteroaryl and aryl, wherein all may be substituted by one or more independently selected from the group consisting of halo, alkyl, lower alkyl, acyl, oxo, hydroxy, hydroxyalkyl, heterocyclic, heteroaryl, aryl, amino, aminoalkyl, cyano, and carboxy.

The compounds and compositions described herein can be administered for the treatment or prophylaxis of an inflammatory disorder. In certain embodiments, the inflammatory disorder is a respiratory disorder. In particular embodiments, the inflammatory disorder is asthma or COPD. In other embodiments the inflammatory disorder is a cardiovascular disorder. Cardiovascular inflammatory disorders include atherosclerosis, post-angioplasty, restenosis, coronary artery diseases, angina, and other cardiovascular diseases. In certain embodiments the disorder is a non-cardiovascular inflammatory disorder such as rheumatoid and osteoarthritis, dermatitis, psoriasis, cystic fibrosis, post transplantation late and chronic solid organ rejection, eczematous dermatitis, Kaposi's sarcoma, multiple sclerosis or is diabetes. In yet another embodiment, the compounds disclosed herein can be selected to treat inflammatory conditions that are mediated by mononuclear leucocytes. In an alternative embodiment, the compounds can be administered to treat small vessel disease that is not treatable by surgery or angioplasty, or other vessel disease in which surgery is not an option. The compounds can also be used to stabilize patients prior to revascularization therapy.

### DETAILED DESCRIPTION OF THE INVENTION

It has been discovered that compounds of Formula (I) inhibit the expression of certain inflammatory cytokines and can be used to treat an inflammatory disease in a patient. Inflammatory disorders include, but are not limited to asthma, atherosclerosis, post-angioplasty, restenosis, coronary artery diseases, angina, as well as other cardiovascular and noncardiovascular inflammatory diseases such as rheumatoid and osteoarthritis, dermatitis, psoriasis, cystic fibrosis, post transplantation late and chronic solid organ rejection, eczematous dermatitis, Kaposi's sarcoma, multiple sclerosis, or proliferative disorders of smooth muscle cells or diabetes.

### Pharmaceutical Compositions and Methods Comprising Compounds of Formula I

In one embodiment, pharmaceutical compositions and methods of treatment or prophylaxis of an inflammatory condition, and in particular asthma, comprising administering to a host in need thereof a compound of Formula I, or a pharmaceutically acceptable salt, ester, prodrug or derivative thereof are provided:

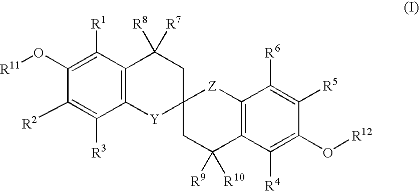

wherein:

Y and Z are independently O, S(O)_{q}, Se(O)_{q} or N(R¹³);

each q is independently 0, 1 or 2;

R¹, R², R³, R⁴, R⁵ and R⁶ are independently selected from the group consisting of hydrogen, halo, C₁-C₆ straight alkyl, C₁-C₆ branched alkyl, C₂-C₆ alkenyl, C₃-C₈ cyclic alkyl, aryl and —OR¹⁴, wherein all may be optionally substituted by a hydroxy group;

R⁷, R⁸, R⁹ and R¹⁰ are independently selected from the group consisting of hydrogen, halo, C₁-C₆ straight alkyl, C₁-C₆ branched alkyl, and C₃-C₈ cyclic alkyl, wherein all may be optionally substituted by a hydroxy group;

R¹¹ and R¹² are independently selected from the group consisting of hydrogen, C₁-C₆ straight alkyl, C₁-C₆ branched alkyl, C₂-C₆ alkenyl, C₃-C₈ cyclic alkyl, and acyl, wherein all may be optionally substituted by one or more independently selected from the group consisting of halo, alkyl, lower alkyl, alkenyl, cycloalkyl, acyl, hydroxy, hydroxyalkyl, carboxy, alkoxy, heterocyclic, heteroaryl, aryl, amino, aminoalkyl, —NR¹⁴R¹⁵, oxo, cyano, alkoxycarbonyl, —OR¹⁶, —C(O)R¹⁶, —C(O)—NH₂, —C(O)—N(H)R¹⁴, —C(O)—N(H)OR¹⁴, —C(O)—NR¹⁴R¹⁵, —NR¹⁵C(O)R¹⁴, —NR¹⁵C(O)NR¹⁴R¹⁵, —OC(O)NR¹⁴R¹⁵, NR¹⁵C(O)OR¹⁶, —S(O)ₙ—R¹⁶, —S(O)₂—NH₂, —S(O)₂—N(H)R¹⁴ and —S(O)₂—NR¹⁴R¹⁵;

Or R¹¹ and R¹² are independently selected from the group consisting of hydrogen, aryl, heteroaryl and heterocycle, wherein all may be optionally substituted by one or more independently selected from the group consisting of halo, alkyl, lower alkyl, alkenyl, cycloalkyl, acyl, hydroxy, hydroxyalkyl, alkoxy, heterocyclic, heteroaryl, aryl, amino, aminoalkyl, —NR¹⁴R¹⁵, oxo, cyano, carboxy, carboxyalkyl, alkoxycarbonyl, —OR¹⁶, —C(O)R¹⁶, —C(O)—NH₂, —C(O)—N(H)R⁴, —C(O)—N(H)OR¹⁴, —C(O)—NR¹⁴R¹⁵, —NR¹⁵C(O)R¹⁴, —NR¹⁵C(O)NR¹⁴R¹⁵, —OC(O)NR¹⁴R¹⁵, —NR¹⁵C(O)OR¹⁶, —S(O)ₙ—R¹⁶, —S(O)₂—NH₂, —S(O)₂—N(H)R¹⁴ and —S(O)₂—NR¹⁴R¹⁵;

each n is independently 0, 1, or 2;

R¹³ is independently selected from the group consisting of hydrogen, hydroxy, C₁-C₆ straight alkyl, C₁-C₆ branched alkyl, C₂-C₆ alkenyl, C₃-C₈ cyclic alkyl, heterocyclic, heteroaryl and aryl, wherein all may be optionally substituted by one or more independently selected from the group consisting of halo, alkyl, lower alkyl, acyl, oxo, hydroxy, hydroxyalkyl, alkoxy, heterocyclic, heteroaryl, cyano, amino, aminoalkyl, and carboxy;

R¹⁴ and R¹⁵ are independently selected from the group consisting of hydrogen, C₁-C₆ straight alkyl, C₁-C₆ branched alkyl, C₂-C₆ alkenyl, C₃-C₈ cyclic alkyl, aryl, heteroaryl, heterocycle, and acyl, wherein all may be optionally substituted by one or more independently selected from the group consisting of halo, alkyl, lower alkyl, alkenyl, cycloalkyl, acyl, hydroxy, hydroxyalkyl, alkoxy heterocyclic, heteroaryl, aryl, amino, aminoalkyl, oxo, cyano, carboxy, carboxyalkyl, alkoxycarbonyl, and —OR¹⁶;

R¹⁴ and R¹⁵ taken together may form a 4- to 12-membered monocyclic, bicyclic, tricyclic or benzofused ring;

R¹⁶ is independently selected from the group consisting of C₁-C₆ straight alkyl, C₁-C₆ branched alkyl, C₂-C₆ alkenyl, C₃-C₈ cyclic alkyl, heterocyclic, heteroaryl and aryl, wherein all may be substituted by one or more independently selected from the group consisting of halo, alkyl, lower alkyl, acyl, oxo, hydroxy, hydroxyalkyl, heterocyclic, heteroaryl, aryl, amino, aminoalkyl, cyano, and carboxy;

with the proviso, that when R¹¹ and R¹² are heteroaryl, R¹¹ and R¹² cannot be 2-furyl.

In one embodiment, the pharmaceutical compositions and methods of treatment or prophylaxis of an inflammatory condition, and in particular asthma, comprises administering to a host in need thereof a compound of Formula I, or a pharmaceutically acceptable salt, ester, prodrug or derivative thereof wherein R¹¹ is hydrogen.

In another subembodiment, Y and Z are independently O or S(O)_{q}. In another embodiment, Y and Z are independently O or S(O)_{q} and q is 0. In another embodiment, Y and Z are each O. In another embodiment, Y and Z are each S(O)_{q} and q is 0. In another embodiment, Y and Z are each Se(O)_{q} and q is 0. In another embodiment, Y and Z are each N(R¹³). In a subembodiment, R¹³ is H or C₁-C₆ straight or branched alkyl.

In one embodiment, R¹, R², R³, R⁴, R⁵ and R⁶ are independently selected from the group consisting of hydrogen, halo, C₁-C₄ straight alkyl, C₁-C₄ branched alkyl, C₂-C₆ alkenyl, C₃-C₆ cyclic alkyl, aryl and —OR⁴, wherein all may be optionally substituted by a hydroxy group. In another embodiment, R¹, R², R³, R⁴, R⁵ and R⁶ are independently selected from the group consisting of hydrogen, halo, C₁-C₄ straight alkyl, C₁-C₄ branched alkyl, C₂-C₆ alkenyl, and C₃-C₆ cyclic alkyl. In another embodiment, R¹, R², R³, R⁴, R⁵ and R⁶ are independently selected from the group consisting of hydrogen, halo, C₁-C₄ straight alkyl, and in particular, H or methyl. In another subembodiment, R¹, R³, R⁴ and R⁶ are hydrogen; and R², R⁵ are C₁-C₄ straight or branched alkyl, for example, methyl, ethyl, propyl, isopropyl, n-butyl, t-butyl, or s-butyl. In another subembodiment, R¹, R³, R⁴ and R⁶ are hydrogen; and R² and R⁵ are C₁-C₄ straight alkyl, C₁-C₄ branched alkyl, or C₂-C₆ alkenyl, wherein all may be optionally substituted by one or more halo groups. In another subembodiment, R¹, R³, R⁴ and R⁶ are hydrogen; and R² and R⁵ are methyl.

In one embodiment, R⁷, R⁸, R⁹ and R¹⁰ are independently selected from the group consisting of hydrogen, halo, C₁-C₄ straight alkyl and C₁-C₄ branched alkyl; wherein all may be optionally substituted by a hydroxy group. In another embodiment, R⁷, R⁸, R⁹ and R¹⁰ are independently selected from the group consisting of C₁-C₄ straight or branched alkyl, for example, methyl, ethyl, propyl, isopropyl, n-butyl, t-butyl, or s-butyl. In another subembodiment, R⁷, R⁸, R⁹ and R¹⁰ are methyl.

In one embodiment, R¹² is hydrogen. In another embodiment, both R¹¹ and R¹² are hydrogen. In another embodiment, at least one of R¹¹ and R¹² is not hydrogen.

In one embodiment, R¹² is C₁-C₆ straight alkyl, C₁-C₆ branched alkyl, C₂-C₆ alkenyl, or C₃-C₈ cyclic alkyl, wherein all may be optionally substituted by one or more independently selected from the group consisting of acyl, hydroxy, C₁-C₄ alkoxy, heterocyclic, heteroaryl, aryl, amino, —NR¹⁴R¹⁵, oxo, cyano, alkoxycarbonyl, —OR¹⁶, —C(O)R¹⁶, —C(O)—NH₂, —C(O)—N(H)R¹⁴, —C(O)—N(H)OR¹⁴, —C(O)—NR¹⁴R¹⁵, —NR¹⁵C(O)R¹⁴, —NR¹⁵C(O)NR¹⁴R¹⁵, —OC(O)NR¹⁴R¹⁵, —NR¹⁵C(O)OR¹⁶, —S(O)ₙ—R¹⁶, —S(O)₂—NH₂, —S(O)₂—N(H)R¹⁴ and —S(O)₂—NR¹⁴R¹⁵. In a particular subembodiment, R¹² is C₁-C₆ straight alkyl, C₁-C₆ branched alkyl, optionally substituted by one or more hydroxy, heteroaryl, or alkoxycarbonyl. In another particular subembodiment, R¹² is C₁-C₄ straight alkyl, C₁-C₄ branched alkyl, optionally substituted by one or more hydroxy, heteroaryl, or alkoxycarbonyl. In another particular subembodiment, R¹² is C₁-C₄ straight alkyl, C₁-C₄ branched alkyl, substituted by one or more hydroxy groups. In another particular subembodiment, R¹² is C₁-C₄ straight alkyl, C₁-C₄ branched alkyl, substituted by one or more heteroaryl which may be optionally substituted. In another particular subembodiment, R¹² is C₁-C₄ straight alkyl, C₁-C₄ branched alkyl, substituted by one or more pyrrolyl, pyrazolyl or imidazolyl, which may be optionally substituted. In another particular subembodiment, R¹² is C₁-C₄ straight alkyl, C₁-C₄ branched alkyl substituted by one or more alkoxycarbonyl.

In embodiment, R¹² is C₁-C₆ straight alkyl, C₁-C₆ branched alkyl, substituted by carboxy, for example, carboxymethyl, 1-carboxyethyl, 2-carboxyethyl, 1-carboxypropyl, 2-carboxypropyl, 3-carboxypropyl, 1-carboxylsopropyl, 2-carboxylsopropyl, 1-carboxy-n-butyl, 2-carboxy-n-butyl, 3-carboxy-n-butyl, 4-carboxy-n-butyl, or 2-carboxy-t-butyl.

In another embodiment, R¹² is hydroxymethyl, 1-hydroxyethyl, 2-hydroxyethyl, 2-hydroxy-2,2-dimethylethyl, 2-hydroxy-1,1-dimethylethyl, 1-hydroxypropyl, 2-hydroxypropyl, 3-hydroxypropyl, 1-hydroxyisopropyl, 2-hydroxyisopropyl, 1-hydroxy-n-butyl, 2-hydroxy-n-butyl, 3-hydroxy-n-butyl, 4-hydroxy-n-butyl, or 2-hydroxy-t-butyl.

In another embodiment, R¹² is 1,2-dihydroxyethyl, 1,2-dihydroxypropyl, 2,3-dihydroxypropyl, 1,3-dihydroxypropyl, 1,2-dihydroxyisopropyl, 1,2-dihydroxy-n-butyl, 1,3-dihydroxy-n-butyl, 1,4-dihydroxy-n-butyl, 2,3-dihydroxy-n-butyl, 2,4-dihydroxy-n-butyl, or 3,4-dihydroxy-n-butyl.

In another embodiment, R¹² is 1,2,3-trihydroxy-n-butyl, 1,2,4-trihydroxy-n-butyl, 1,3,4-trihydroxy-n-butyl, or 2,3,4-trihydroxy-n-butyl.

In another embodiment, R¹² is C₁-C₆ straight alkyl, C₁-C₆ branched alkyl, substituted by a substituted or unsubstituted heteroaryl or heterocycle, for example, pyrazole, imidazole, methyl-pyrazole, dimethylpyrazole, 3-hydroxymethyl-5-methyl-pyrazole, 5-hydroxymethyl-3-methyl-pyrazole.

In another embodiment, R¹² is optionally substituted C₂-C₆ alkenyl, for example, ethenyl, propenyl, 1-butenyl, 2-butenyl or 3-butenyl.

In another embodiment, R¹² is selected from the group consisting of aryl, heteroaryl and heterocycle, wherein all may be optionally substituted by one or more independently selected from the group consisting of halo, alkyl, lower alkyl, alkenyl, cycloalkyl, acyl, hydroxy, hydroxyalkyl, alkoxy, heterocyclic, heteroaryl, aryl, amino, aminoalkyl, —NR¹⁴R¹⁵, oxo, cyano, carboxy, carboxyalkyl, alkoxycarbonyl, —OR¹⁶, —C(O)R¹⁶, —C(O)—NH₂, —C(O)—N(H)R¹⁴, —C(O)—N(H)OR¹⁴, —C(O)—NR¹⁴R¹⁵, —NR¹⁵C(O)R¹⁴, —NR¹⁵C(O)NR¹⁴R¹⁵, —OC(O)NR¹⁴R¹⁵, —NR¹⁵C(O)OR¹⁶, —S(O)ₙ—R¹⁶, —S(O)₂—NH₂, —S(O)₂—N(H)R¹⁴ and —S(O)₂—NR¹⁴R¹⁵. In a particular subembodiment, R¹² is selected from the group consisting of aryl, heteroaryl and heterocycle, wherein all may be optionally substituted by one or more independently selected from the group consisting of alkyl, acyl, hydroxy, hydroxyalkyl, alkoxy, carboxy, carboxyalkyl, or alkoxycarbonyl. In another subembodiment, R¹² is a pyrrolyl, pyrazolyl or imidazolyl group, all of which may be optionally substituted.

In one embodiment, the compound is selected from the group consisting of:

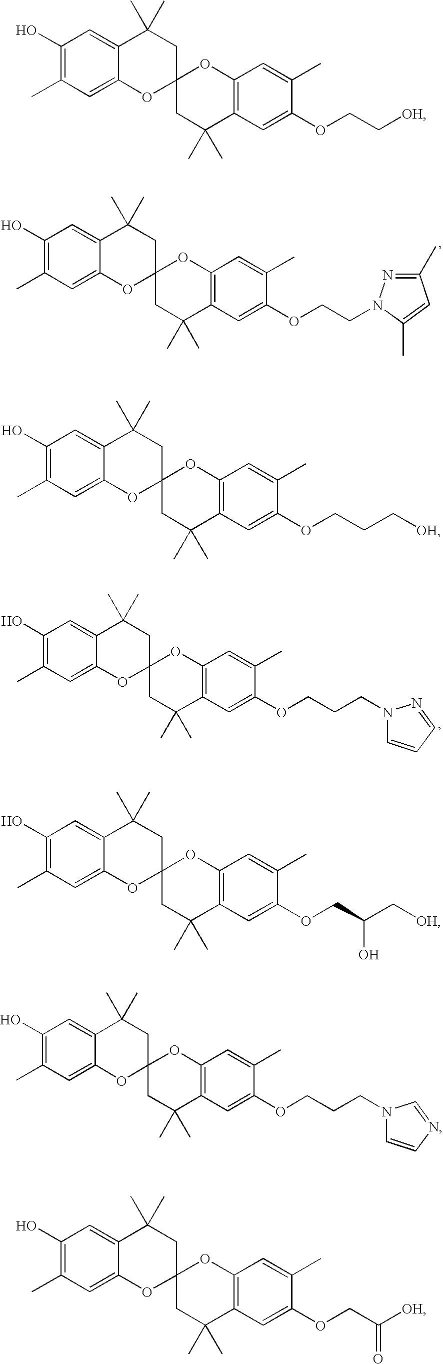

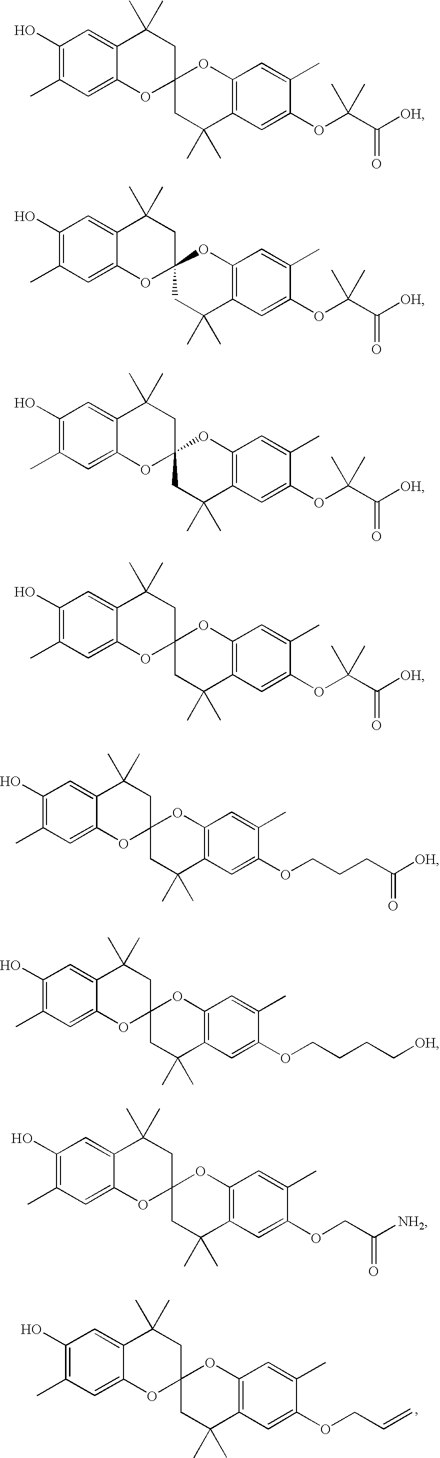

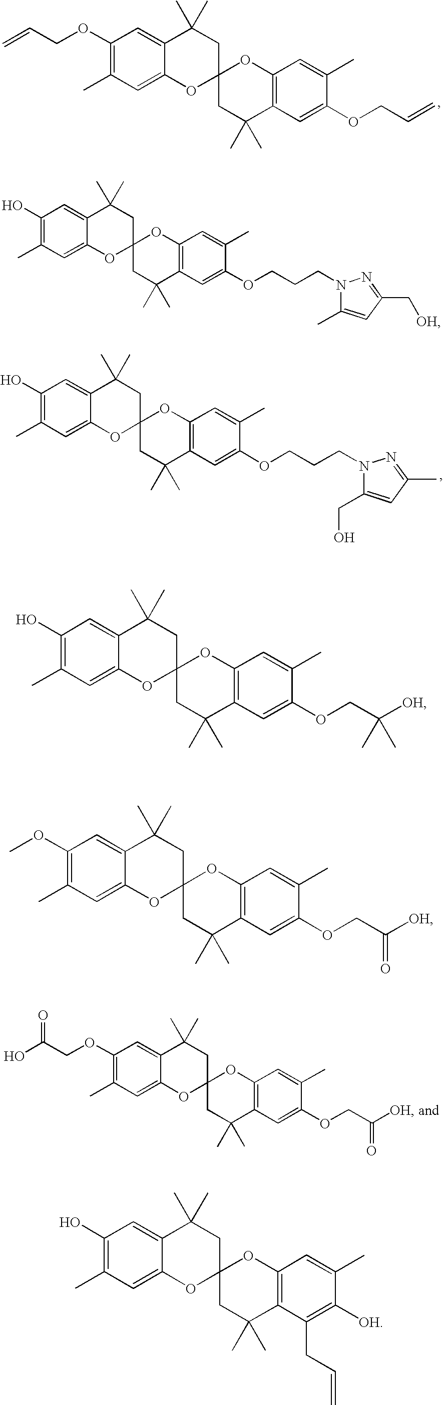

In a subembodiment of any of the foregoing formula or embodiments, the pharmaceutical compositions and methods of treatment or prophylaxis of an inflammatory condition, and in particular asthma, comprises administering to a host in need thereof one enantiomer or one stereoisomer of a compound of Formula I, or a pharmaceutically acceptable salt, ester, prodrug or derivative thereof.

In certain embodiments, the compounds are present as enantiomers. In particular embodiments, the compound is present as a racemic mixture. The enantiomer can be named by the configuration at the chiral center, such as R or S. In certain embodiments, the compound is present as a racemic mixture of R- and S- enantiomers. In certain embodiments, the compound is present as a mixture of two enantiomers. In one embodiment, the mixture has an enantiomeric excess in R. In one embodiment, the mixture has an enantiomeric excess in S. In certain other embodiments, the compound is in an enantiomeric excess of the R- or S- enantiomer. The enantiomeric excess can be 51% or more, such as 51% or more, 55% or more, 60% or more, 65% or more, 70% or more, 75% or more, 80% or more, 85% or more, 90% or more, 95% or more, 98% or more, or 99% or more in the single enantiomer. The enantiomeric excess can be 51% or more, such as 51% or more, 55% or more, 60% or more, 65% or more, 70% or more, 75% or more, 80% or more, 85% or more, 90% or more, 95% or more, 98% or more, or 99% or more in the R enantiomer. The enantiomeric excess can be 51% or more, such as 51% or more, 55% or more, 60% or more, 65% or more, 70% or more, 75% or more, 80% or more, 85% or more, 90% or more, 95% or more, 98% or more, or 99% or more in the S enantiomer.

In other embodiments, the compound is substantially in the form of a single enantiomer. In some embodiments, the compound is present substantially in the form of the R enantiomer. In some embodiments, the compound is present substantially in the form of the S enantiomer. The phrase “substantially in the form of a single enantiomer” is intended to mean at least 70% or more in the form of a single enantiomer, for example 70% or more, 75% or more, 80% or more, 85% or more, 90% or more, 95% or more, 98% or more, or 99% or more in either the R or S enantiomer.

The enantiomer can be named by the direction in which it rotates the plane of polarized light. If it rotates the light clockwise as seen by the viewer towards whom the light is traveling, the isomer can be labeled (+) and if it rotates the light counterclockwise, the isomer can be labeled (−). In certain embodiments, the compound is present as a racemic mixture of (+) and (−) isomers. In certain embodiments, the compound is present as a mixture of two isomers. In one embodiment, the mixture has an excess in (+). In one embodiment, the mixture has an excess in (−). In certain other embodiments, the compound is in an excess of the (+) or (−) isomer. The isomeric excess can be 51% or more, such as 51% or more, 55% or more, 60% or more, 65% or more, 70% or more, 75% or more, 80% or more, 85% or more, 90% or more, 95% or more, 98% or more, or 99% or more in the (+) isomer. The enantiomeric excess can be 51% or more, such as 51% or more, 55% or more, 60% or more, 65% or more, 70% or more, 75% or more, 80% or more, 85% or more, 90% or more, 95% or more, 98% or more, or 99% or more in the (−) isomer.

In other embodiments, the compound is substantially in the form of a single optical isomer. In some embodiments, the compound is present substantially in the form of the (+) isomer. In other embodiments, the compound is present substantially in the form of the (−) isomer. The phrase “substantially in the form of a single optical isomer” is intended to mean at least 70% or more in the form of a single isomer, for example 70% or more, 75% or more, 80% or more, 85% or more, 90% or more, 95% or more, 98% or more, or 99% or more of either the (+) or (−) isomer.

In certain embodiments, the compound has two or more chiral or stereogenic carbons. In certain embodiments, the compound is a compound of Formula I wherein R¹² comprises a substituent with one or more chiral or stereogenic carbons.

In certain embodiments the compound of any of the foregoing formula or embodiments, is a diastereomer. In certain embodiments, the compound is substantially in the form of one diastereomer. In certain embodiments, the compound is substantially in the form of two diastereomers.

In particular embodiments, the pharmaceutical compositions and methods of treatment or prophylaxis of an inflammatory condition, and in particular asthma, comprises administering to a host in need thereof one diastereomer of a compound of Formula I, or a pharmaceutically acceptable salt, ester, prodrug or derivative thereof.

In a subembodiment of any of the foregoing formula or embodiments, the pharmaceutical compositions and methods of treatment or prophylaxis of an inflammatory condition, and in particular asthma, comprises administering to a host in need thereof one diastereomer, or a mixture of two or more diastereomers, of a compound of Formula I, or a pharmaceutically acceptable salt, ester, prodrug or derivative thereof.

In certain embodiments, the compounds are present as diastereomers. In particular embodiments, the compound is present as a mixture of diastereomers. The diastereomers can be named by the configuration at each of the chiral centers, such as (R,R) or (R,S). In certain embodiments, the compound is present as a mixture of two diastereomers. In certain embodiments, the compound is present as a mixture of four diastereomers. In certain embodiments, the compound is present as a mixture of two or more diastereomers. In one embodiment, the mixture has one diastereomer in excess.

In other embodiments, the compound is substantially in the form of one diastereomer. The phrase “substantially in the form of one diastereomer” is intended to mean at least 70% or more in the form of the diastereomer, for example 70% or more, 75% or more, 80% or more, 85% or more, 90% or more, 95% or more, 98% or more, or 99% or more.

In certain embodiments the compound of any of the foregoing formula or embodiments, is a diastereomer. In particular embodiments, the pharmaceutical compositions and methods of treatment or prophylaxis of an inflammatory condition, and in particular asthma, comprises administering to a host in need thereof one diastereomer of a compound of Formula I, or a pharmaceutically acceptable salt, ester, prodrug or derivative thereof.

### Compounds, Pharmaceutical Compositions and Methods Comprising Compounds of Formula II

In one embodiment, compounds, pharmaceutical compositions and methods of treatment or prophylaxis of an inflammatory condition, and in particular asthma, comprising administering to a host in need thereof a compound of Formula II, or a pharmaceutically acceptable salt, ester, prodrug or derivative thereof are provided:

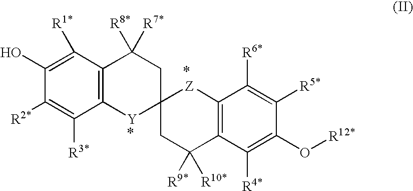

wherein:

Y* and Z* are independently O, S(O)_{q}, Se(O)_{q} or N(R¹³);

each q is independently 0, 1 or 2;

R^{1*}, R^{2*}, R^{3*}, R^{4*}, R^{15*} and R^{6*} are independently selected from the group consisting of hydrogen, halo, C₁-C₆ straight alkyl, C₁-C₆ branched alkyl, C₂-C₆ alkenyl, C₃-C₈ cyclic alkyl, aryl and —OR^{14*}, wherein all may be optionally substituted by a hydroxy group;

R^{7*}, R^{8*}, R^{9*} and R^{10*} are independently selected from the group consisting of hydrogen, halo, C₁-C₆ straight alkyl, C₁-C₆ branched alkyl, and C₃-C₈ cyclic alkyl, wherein all may be optionally substituted by a hydroxy group;

R^{12*} is selected from the group consisting of C₁-C₆ straight alkyl, hydroxy-C₁-C₆ straight alkyl, polyhydroxy-C₁-C₆ straight alkyl, carboxy-C₁-C₆ straight alkyl, carboxy-C₃-C₆ branched alkyl, carboxy-C₂-C₆ alkenyl, carboxy-C₃-C₈ cyclic alkyl, (C(O)NHR^{13*})—C₁-C₆ straight alkyl, (C(O)NHR^{13*})—C₃-C₆ branched alkyl, (C(O)NHR^{13*})—C₂-C₆ alkenyl, (C(O)NHR^{13*})—C₃-C₈ cyclic alkyl, heteroaralkyl, heterocyclicalkyl, and aralkyl, wherein all may be optionally substituted by one or more independently selected from the group consisting of halo, alkyl, lower alkyl, alkenyl, cycloalkyl, acyl, hydroxy, hydroxyalkyl, heterocyclic, heteroaryl, aryl, amino, aminoalkyl, —NR^{14*}R^{15*}, oxo, cyano, carboxy, carboxyalkyl, alkoxycarbonyl, —OR^{16*}, —C(O)R^{16*}, —C(O)—NH₂, —C(O)—N(H)R^{14*}, —C(O)—N(H)OR^{14*}, —C(O)NR^{14*}R^{15*}, —NR^{15*}C(O)R^{14*}, —NR^{15*}C(O)NR^{14*}R^{15*}, —OC(O)NR^{14*}R^{15*}, —NR^{15*}C(O)OR^{16*}, —S(O)ₙ—R^{16*}, —S(O)₂—NH₂, —S(O)₂—N(H)R^{14*} and —S(O)₂—NR^{14*}R^{15*};

each n is independently 0, 1, or 2;

R^{13*} is selected from the group consisting of hydrogen, hydroxy, C₁-C₆ straight alkyl, C₁-C₆ branched alkyl, C₂-C₆ alkenyl, C₃-C₈ cyclic alkyl, heterocyclic, heteroaryl and aryl, wherein all may be optionally substituted by one or more independently selected from the group consisting of halo, alkyl, lower alkyl, acyl, oxo, hydroxy, hydroxyalkyl, heterocyclic, heteroaryl, cyano, amino, aminoalkyl, and carboxy;

R^{14*} and R^{15*} are independently selected from the group consisting of hydrogen, C₁-C₆ straight alkyl, C₁-C₆ branched alkyl, C₂-C₆ alkenyl, C₃-C₈ cyclic alkyl, aryl, heteroaryl, heterocycle, and acyl, wherein all may be optionally substituted by one or more independently selected from the group consisting of halo, alkyl, lower alkyl, alkenyl, cycloalkyl, acyl, hydroxy, hydroxyalkyl, heterocyclic, heteroaryl, aryl, amino, aminoalkyl, oxo, cyano, carboxy, carboxyalkyl, alkoxycarbonyl, and —OR^{16*};

R^{14*} and R^{15*} taken together may form a 4- to 12-membered monocyclic, bicyclic, tricyclic or benzofused ring;

R^{16*} is independently selected from the group consisting of C₁-C₆ straight alkyl, C₁-C₆ branched alkyl, C₂-C₆ alkenyl, C₃-C₈ cyclic alkyl, heterocyclic, heteroaryl and aryl, wherein all may be substituted by one or more independently selected from the group consisting of halo, alkyl, lower alkyl, acyl, oxo, hydroxy, hydroxyalkyl, heterocyclic, heteroaryl, aryl, amino, aminoalkyl, cyano, and carboxy.

In one embodiment, the compound is a compound of formula II wherein R^{1*}, R^{2*}, R^{3*}, R^{4*}, R^{15*} and R^{6*} are independently selected from the group consisting of hydrogen, halo, C₁-C₄ straight alkyl, C₁-C₄ branched alkyl, C₂-C₆ alkenyl, aryl and —OR^{14*}.

In another embodiment, R^{7*}, R^{8*}, R^{9*} and R^{10*} are independently selected from the group consisting of hydrogen, halo, C₁-C₄ straight alkyl, and C₁-C₄ branched alkyl.

In another subembodiment, Y* and Z* are independently O or S(O)_{q}. In another embodiment, Y* and Z* are independently O or S(O)_{q} and q is 0. In another embodiment, Y* and Z are each O. In another embodiment, Y* and Z* are each S(O)_{q} and q is 0. In another embodiment, Y* and Z* are each Se(O)_{q} and q is 0. In another embodiment, Y* and Z* are each N(R¹³). In another embodiment, R¹³ is H or C₁-C₆ straight or branched alkyl.

In one embodiment, compounds, pharmaceutical compositions and methods of treatment or prophylaxis of an inflammatory condition, and in particular asthma, comprising administering to a host in need thereof a compound of Formula III, or a pharmaceutically acceptable salt, ester, prodrug or derivative thereof are provided:

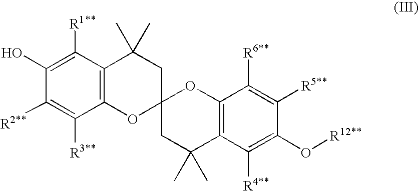

wherein:

R^{1**}, R^{2**}, R^{3**}, R^{4**}, R^{15**} and R^{6**} are independently selected from the group consisting of hydrogen, halo, C₁-C₄ straight alkyl, C₁-C₄ branched alkyl, C₂-C₆ alkenyl, aryl and —OR^{4**};

R^{12**}is selected from the group consisting of C₁-C₆ straight alkyl, hydroxy-C₁-C₆ straight alkyl, polyhydroxy-C₁-C₆ straight alkyl, carboxy-C₁-C₆ straight alkyl, carboxy-C₃-C₆ branched alkyl, carboxy-C₂-C₆ alkenyl, carboxy-C₃-C₈ cyclic alkyl, (C(O)NHR^{13**})—C₁-C₆ straight alkyl, (C(O)NHR^{3**})—C₃-C₆ branched alkyl, (C(O)NHR^{13**})—C₂-C₆ alkenyl, (C(O)NHR^{13**})—C₃-C₈ cyclic alkyl, heteroaralkyl, heterocyclicalkyl, and aralkyl, wherein all may be optionally substituted by one or more independently selected from the group consisting of halo, alkyl, lower alkyl, alkenyl, cycloalkyl, acyl, hydroxy, hydroxyalkyl, heterocyclic, heteroaryl, aryl, amino, aminoalkyl, —NR^{14**}R^{15**}, oxo, cyano, carboxy, carboxyalkyl, alkoxycarbonyl, —OR^{16**}, —C(O)R^{16**}, —C(O)—NH₂, —C(O)—N(H)R^{14**}, —C(O)—N(H)OR^{14**}, —C(O)—NR^{14**}R^{15**}, NR^{15**}C(O)R^{14**}, —NR^{15**}C(O)NR^{14**}R^{15**}, —OC(O)NR^{14**}R^{15**}, —NR^{15**}C(O)OR^{16**}, —S(O)ₙ—R^{16**}, —S(O)₂—NH₂, —S(O)₂—N(H)R^{14**} and —S(O)₂—NR^{14**}R^{15**};

each n is independently 0, 1, or 2;

R^{13**}is selected from the group consisting of hydrogen, hydroxy, C₁-C₆ straight alkyl, C₁-C₆ branched alkyl, C₂-C₆ alkenyl, C₃-C₈ cyclic alkyl, heterocyclic, heteroaryl and aryl, wherein all may be optionally substituted by one or more independently selected from the group consisting of halo, alkyl, lower alkyl, acyl, oxo, hydroxy, hydroxyalkyl, heterocyclic, heteroaryl, cyano, amino, aminoalkyl, and carboxy;

R^{14**}and R^{15**}are independently selected from the group consisting of hydrogen, C₁-C₆ straight alkyl, C₁-C₆ branched alkyl, C₂-C₆ alkenyl, C₃-C₈ cyclic alkyl, aryl, heteroaryl, heterocycle, and acyl, wherein all may be optionally substituted by one or more independently selected from the group consisting of halo, alkyl, lower alkyl, alkenyl, cycloalkyl, acyl, hydroxy, hydroxyalkyl, heterocyclic, heteroaryl, aryl, amino, aminoalkyl, oxo, cyano, carboxy, carboxyalkyl, alkoxycarbonyl, and —OR¹⁶;

R^{14**}and R^{15**}taken together may form a 4- to 12-membered monocyclic, bicyclic, tricyclic or benzofused ring;

R^{16**}is independently selected from the group consisting of C₁-C₆ straight alkyl, C₁-C₆ branched alkyl, C₂-C₆ alkenyl, C₃-C₈ cyclic alkyl, heterocyclic, heteroaryl and aryl, wherein all may be substituted by one or more independently selected from the group consisting of halo, alkyl, lower alkyl, acyl, oxo, hydroxy, hydroxyalkyl, heterocyclic, heteroaryl, aryl, amino, aminoalkyl, cyano, and carboxy.

In one embodiment, compounds, pharmaceutical compositions and methods of treatment or prophylaxis of an inflammatory condition, and in particular asthma, comprising administering to a host in need thereof a compound of Formula IV, or a pharmaceutically acceptable salt, ester, prodrug or derivative thereof are provided:

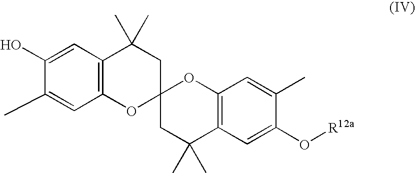

wherein:

R^{12a} is selected from the group consisting of carboxy-C₁-C₆ straight alkyl, carboxy-C₃-C₆ branched alkyl, carboxy-C₂-C₆ alkenyl, carboxy-C₃-C₈ cyclic alkyl, (C(O)NHR^{13a})—C₁-C₆ straight alkyl, (C(O)NHR^{13a})—C₃-C₆ branched alkyl, (C(O)NHR^{13a})—C₂-C₆ alkenyl, (C(O)NHR^{13a})—C₃-C₈ cyclic alkyl, heteroaralkyl, heterocyclicalkyl, and aralkyl, wherein all may be optionally substituted by one or more independently selected from the group consisting of halo, alkyl, lower alkyl, alkenyl, cycloalkyl, acyl, hydroxy, hydroxyalkyl, heterocyclic, heteroaryl, aryl, amino, aminoalkyl, —NR^{14a}R^{15a}, oxo, cyano, carboxy, carboxyalkyl, alkoxycarbonyl, —OR^{16a}, —C(O)R^{16a}, —C(O)—NH₂, —C(O)—N(H)R^{14a}, —C(O)—N(H)OR^{14a}, —C(O)—NR^{14a}R^{15a}, NR^{15a}C(O)R^{14a}, —NR^{15a}C(O)NR^{14a}R^{15a}, —OC(O)NR^{14a}R^{15a}, NR^{15a}C(O)OR^{16a}, —S(O)ₙ—R^{16a}, —S(O)₂—NH₂, —S(O)₂—N(H)R^{14a} and —S(O)₂—NR^{14a}R^{15a}; or

R^{12a} is selected from the group consisting of C₁-C₆ straight alkyl, C₃-C₆ branched alkyl, C₂-C₆ alkenyl, C₃-C₈ cyclic alkyl, hydroxy-C₁-C₆ straight alkyl, hydroxy-C₃-C₆ branched alkyl, hydroxy-C₂-C₆ alkenyl, hydroxy-C₃-C₈ cyclic alkyl, wherein all may be optionally substituted by one or more independently selected from the group consisting of halo, alkyl, lower alkyl, alkenyl, cycloalkyl, acyl, hydroxy, hydroxyalkyl, heterocyclic, heteroaryl, aryl, amino, aminoalkyl, —NR^{14a}R^{15a}, oxo, cyano, carboxy, carboxyalkyl, alkoxycarbonyl, OR^{16a}, —C(O)R^{16a}, —C(O)—NH₂, —C(O)—N(H)R^{14a}, —C(O)—N(H)OR^{14a}, —C(O)—NR^{14a}R^{15a} NR^{15a}C(O)R^{14a}, —NR^{15a}C(O)NR^{14a}R^{15a}, —OC(O)NR^{14a}R^{15a}, —NR^{15a}C(O)OR^{16a}, —S(O)ₙ—R^{16a}, —S(O)₂—NH₂, —S(O)₂—N(H)R^{14a} and —S(O)₂—NR^{14a}R^{15a};

each n is independently 0, 1, or 2;

R^{13a} is selected from the group consisting of hydrogen, hydroxy, C₁-C₆ straight alkyl, C₁-C₆ branched alkyl, C₂-C₆ alkenyl, C₃-C₈ cyclic alkyl, heterocyclic, heteroaryl and aryl, wherein all may be optionally substituted by one or more independently selected from the group consisting of halo, alkyl, lower alkyl, acyl, oxo, hydroxy, hydroxyalkyl, heterocyclic, heteroaryl, cyano, amino, aminoalkyl, and carboxy;

R^{14a} and R^{15a} are independently selected from the group consisting of hydrogen, C₁-C₆ straight alkyl, C₁-C₆ branched alkyl, C₂-C₆ alkenyl, C₃-C₈ cyclic alkyl, aryl, heteroaryl, heterocycle, and acyl, wherein all may be optionally substituted by one or more independently selected from the group consisting of halo, alkyl, lower alkyl, alkenyl, cycloalkyl, acyl, hydroxy, hydroxyalkyl, heterocyclic, heteroaryl, aryl, amino, aminoalkyl, oxo, cyano, carboxy, carboxyalkyl, alkoxycarbonyl, and —OR^{16a};

R^{14a} and R^{15a} taken together may form a 4- to 12-membered monocyclic, bicyclic, tricyclic or benzofused ring;

R^{16a} is selected from the group consisting of C₁-C₆ straight alkyl, C₁-C₆ branched alkyl, C₂-C₆ alkenyl, C₃-C₈ cyclic alkyl, heterocyclic, heteroaryl and aryl, wherein all may be substituted by one or more independently selected from the group consisting of halo, alkyl, lower alkyl, acyl, oxo, hydroxy, hydroxyalkyl, heterocyclic, heteroaryl, aryl, amino, aminoalkyl, cyano, and carboxy.

In one embodiment, the compound of Formula IV is a compound wherein R^{12a} is selected from the group consisting of carboxy-C₁-C₆ straight alkyl, carboxy-C₃-C₆ branched alkyl, carboxy-C₃-C₈ cyclic alkyl, (C(O)NHR^{13a})—C₁-C₆ straight alkyl, (C(O)NHR^{13a})—C₃-C₆ branched alkyl, (C(O)NHR^{13a})—C₃-C₈ cyclic alkyl, heteroaralkyl, and heterocyclicalkyl, wherein all may be optionally substituted by one or more independently selected from the group consisting of halo, lower alkyl, cycloalkyl, acyl, hydroxy, hydroxyalkyl, heterocyclic, heteroaryl, amino, aminoalkyl, —NR^{14a}R^{15a}, oxo, cyano, carboxy, carboxyalkyl, alkoxycarbonyl, OR^{16a}, —C(O)R^{16a}, —C(O)—NH₂, —C(O)—N(H)R^{14a}, —C(O)—N(H)OR^{14a}, —C(O)—NR^{14a}R^{15a} and —NR^{15a}C(O)R^{14a}; or

R^{12a} is selected from the group consisting of C₁-C₆ straight alkyl, C₃-C₆ branched alkyl, C₂-C₆ alkenyl, C₃-C₈ cyclic alkyl, hydroxy-C₁-C₆ straight alkyl, hydroxy-C₃-C₆ branched alkyl, hydroxy-C₂-C₆ alkenyl, hydroxy-C₃-C₈ cyclic alkyl, wherein all may be optionally substituted by one or more independently selected from the group consisting of halo, lower alkyl, cycloalkyl, acyl, hydroxy, hydroxyalkyl, heterocyclic, heteroaryl, amino, aminoalkyl, —NR^{14a}R^{15a}, oxo, cyano, carboxy, carboxyalkyl, alkoxycarbonyl, —OR^{16a}, —C(O)R^{16a}, —C(O)—NH₂, —C(O)—N(H)R^{14a}, —C(O)—N(H)OR^{14a}, —C(O)—NR^{14a}R^{15a} and —NR^{15a}C(O)R^{14a};

R^{13a} is selected from the group consisting of hydrogen, hydroxy, heterocyclic and heteroaryl, wherein all may be optionally substituted by one or more independently selected from the group consisting of halo, alkyl, lower alkyl, acyl, oxo, hydroxy, hydroxyalkyl, heterocyclic, heteroaryl, cyano, amino, aminoalkyl, and carboxy;

R^{14a} and R^{15a} are independently selected from the group consisting of hydrogen, C₁-C₆ straight alkyl, C₁-C₆ branched alkyl, heteroaryl, heterocycle, and acyl, wherein all may be optionally substituted by one or more independently selected from the group consisting of halo, alkyl, lower alkyl, alkenyl, cycloalkyl, acyl, hydroxy, hydroxyalkyl, heterocyclic, heteroaryl, aryl, amino, aminoalkyl, oxo, cyano, carboxy, carboxyalkyl, alkoxycarbonyl, and OR^{16a}; and

R^{16a} is independently selected from the group consisting of C₁-C₄ straight alkyl, C₁-C₄ branched alkyl, heterocyclic and heteroaryl, wherein all may be substituted by one or more independently selected from the group consisting of halo, alkyl, lower alkyl, acyl, oxo, hydroxy, hydroxyalkyl, heterocyclic, heteroaryl, aryl, amino, aminoalkyl, cyano, and carboxy.

In another embodiment, the compound of Formula IV is a compound wherein R^{12a} is selected from the group consisting of carboxy-C₁-C₄ straight alkyl, carboxy-C₃-C₆ branched alkyl, (C(O)NHR^{13a})—C₁-C₄ straight alkyl and (C(O)NHR^{13a})—C₃-C₆ branched alkyl, wherein all may be optionally substituted by one or more independently selected from the group consisting of halo, lower alkyl, acyl, hydroxy, hydroxyalkyl, heterocyclic, heteroaryl, amino, aminoalkyl, oxo, cyano, and alkoxycarbonyl; and

R^{13a} is selected from the group consisting of hydrogen, hydroxy, heterocyclic and heteroaryl, wherein all may be optionally substituted by one or more independently selected from the group consisting of halo, alkyl, lower alkyl, acyl, oxo, hydroxy, hydroxyalkyl, heterocyclic, heteroaryl, cyano, amino, aminoalkyl, and carboxy.

In another embodiment, the compound of Formula IV is a compound wherein R^{12a} is selected from the group consisting of carboxy-C₁-C₄ straight alkyl and carboxy-C₃-C₆ branched alkyl, wherein all may be optionally substituted by one or more independently selected from the group consisting of halo, lower alkyl, acyl, hydroxy, hydroxyalkyl, heterocyclic, heteroaryl, and amino.

In another embodiment, the compound of Formula IV is a compound wherein R^{12a} is selected from the group consisting of C₁-C₆ straight alkyl, C₃-C₆ branched alkyl, C₂-C₆ alkenyl, C₃-C₈ cyclic alkyl, hydroxy-C₁-C₆ straight alkyl, hydroxy-C₃-C₆ branched alkyl, hydroxy-C₂-C₆ alkenyl, hydroxy-C₃-C₈ cyclic alkyl, wherein all may be optionally substituted by one or more independently selected from the group consisting of halo, lower alkyl, acyl, hydroxy, hydroxyalkyl, heterocyclic, heteroaryl, amino, aminoalkyl, oxo, cyano, and alkoxycarbonyl; and

R^{13a} is selected from the group consisting of hydrogen, hydroxy, heterocyclic and heteroaryl, wherein all may be optionally substituted by one or more independently selected from the group consisting of halo, alkyl, lower alkyl, acyl, oxo, hydroxy, hydroxyalkyl, heterocyclic, heteroaryl, cyano, amino, aminoalkyl, and carboxy.

In another embodiment, the compound of Formula IV is a compound wherein R^{12a} is selected from the group consisting of hydroxy-C₁-C₄ straight alkyl and hydroxy-C₃-C₆ branched alkyl, wherein all may be optionally substituted by one or more independently selected from the group consisting of halo, lower alkyl, acyl, hydroxy, hydroxyalkyl, heterocyclic, heteroaryl, and amino.

In another embodiment, the compound of Formula IV is a compound wherein R^{12a} is selected from the group consisting of C₁-C₄ straight alkyl and C₃-C₆ branched alkyl, wherein all may be optionally substituted by one or more independently selected from the group consisting of halo, lower alkyl, acyl, hydroxy, hydroxyalkyl, heterocyclic, heteroaryl, and amino.

In one embodiment, compounds, pharmaceutical compositions and methods of treatment or prophylaxis of an inflammatory condition, and in particular asthma, comprising administering to a host in need thereof a compound of Formula V, or a pharmaceutically acceptable salt, ester, prodrug or derivative thereof are provided:

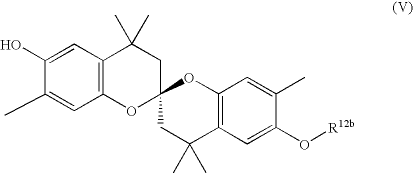

wherein:

R^{12b} is selected from the group consisting of carboxy-C₁-C₆ straight alkyl, carboxy-C₃-C₆ branched alkyl, carboxy-C₂-C₆ alkenyl, carboxy-C₃-C₈ cyclic alkyl, (C(O)NHR^{13b})—C₁-C₆ straight alkyl, (C(O)NHR^{13b})—C₃-C₆ branched alkyl, (C(O)NHR^{13b})—C₂-C₆ alkenyl, (C(O)NHR^{13b})—C₃-C₈ cyclic alkyl, heteroaralkyl, heterocyclicalkyl, and aralkyl, wherein all may be optionally substituted by one or more independently selected from the group consisting of halo, alkyl, lower alkyl, alkenyl, cycloalkyl, acyl, hydroxy, hydroxyalkyl, heterocyclic, heteroaryl, aryl, amino, aminoalkyl, —NR^{14b}R^{15b}, oxo, cyano, carboxy, carboxyalkyl, alkoxycarbonyl, —OR^{16b}, —C(O)R^{16b}, —C(O)—NH₂, —C(O)—N(H)R^{14b}, —C(O)—N(H)OR^{14b}, —C(O)—NR^{14b}R^{15b}, NR^{15b}C(O)R^{14b}, —NR^{15b}C(O)NR^{14b}R^{15b}, —OC(O)NR^{14b}R^{15b}, —NR^{15b}C(O)OR^{16b}, —S(O)ₙ—R^{16b}, —S(O)₂—NH₂, —S(O)₂—N(H)R^{14b} and —S(O)₂—NR^{14b}R^{15b}; or

or

R^{12b} is selected from the group consisting of C₁-C₆ straight alkyl, C₃-C₆ branched alkyl, C₂-C₆ alkenyl, C₃-C₈ cyclic alkyl, hydroxy-C₁-C₆ straight alkyl, hydroxy-C₃-C₆ branched alkyl, hydroxy-C₂-C₆ alkenyl, hydroxy-C₃-C₈ cyclic alkyl, wherein all may be optionally substituted by one or more independently selected from the group consisting of halo, alkyl, lower alkyl, alkenyl, cycloalkyl, acyl, hydroxy, hydroxyalkyl, heterocyclic, heteroaryl, aryl, amino, aminoalkyl, —NR^{14b}R^{15b}, oxo, cyano, carboxy, carboxyalkyl, alkoxycarbonyl, —OR^{16b}, —C(O)R^{16b}, —C(O)—NH₂, —C(O)—N(H)R^{14b}, —C(O)—N(H)OR^{14b}, —C(O)—NR^{14b}R^{15b}, NR^{15b}C(O)R^{14b}, NR^{15b}C(O)NR^{14b}R^{15b}, —OC(O)NR^{14b}R^{15b}, —NR^{15b}C(O)OR^{16b}, —S(O)ₙ—R^{16b}—S(O)₂—NH₂, —S(O)₂—N(H)R^{14b} and —S(O)₂—NR^{14b}R^{15b};

each n is independently 0, 1, or 2;

R^{13b} is selected from the group consisting of hydrogen, hydroxy, C₁-C₆ straight alkyl, C₁-C₆ branched alkyl, C₂-C₆ alkenyl, C₃-C₈ cyclic alkyl, heterocyclic, heteroaryl and aryl, wherein all may be optionally substituted by one or more independently selected from the group consisting of halo, alkyl, lower alkyl, acyl, oxo, hydroxy, hydroxyalkyl, heterocyclic, heteroaryl, cyano, amino, aminoalkyl, and carboxy;

R^{14b} and R^{15b} are independently selected from the group consisting of hydrogen, C₁-C₆ straight alkyl, C₁-C₆ branched alkyl, C₂-C₆ alkenyl, C₃-C₈ cyclic alkyl, aryl, heteroaryl, heterocycle, and acyl, wherein all may be optionally substituted by one or more independently selected from the group consisting of halo, alkyl, lower alkyl, alkenyl, cycloalkyl, acyl, hydroxy, hydroxyalkyl, heterocyclic, heteroaryl, aryl, amino, aminoalkyl, oxo, cyano, carboxy, carboxyalkyl, alkoxycarbonyl, and —OR^{16b};

R^{14b} and R^{15b} taken together may form a 4- to 12-membered monocyclic, bicyclic, tricyclic or benzofused ring;

R^{16b} is selected from the group consisting of C₁-C₆ straight alkyl, C₁-C₆ branched alkyl, C₂-C₆ alkenyl, C₃-C₈ cyclic alkyl, heterocyclic, heteroaryl and aryl, wherein all may be substituted by one or more independently selected from the group consisting of halo, alkyl, lower alkyl, acyl, oxo, hydroxy, hydroxyalkyl, heterocyclic, heteroaryl, aryl, amino, aminoalkyl, cyano, and carboxy.

In one embodiment, the compound of Formula IV is a compound wherein R^{12b} is selected from the group consisting of carboxy-C₁-C₆ straight alkyl, carboxy-C₃-C₆ branched alkyl, carboxy-C₃-C₈ cyclic alkyl, (C(O)NHR^{13b})—C₁-C₆ straight alkyl, (C(O)NHR^{13b})—C₃-C₆ branched alkyl, (C(O)NHR^{13b})—C₃-C₈ cyclic alkyl, heteroaralkyl, and heterocyclicalkyl, wherein all may be optionally substituted by one or more independently selected from the group consisting of halo, lower alkyl, cycloalkyl, acyl, hydroxy, hydroxyalkyl, heterocyclic, heteroaryl, amino, aminoalkyl, —NR^{14b}R^{15b}, oxo, cyano, carboxy, carboxyalkyl, alkoxycarbonyl, —OR^{16b}, —C(O)R^{16b}, —C(O)—NH₂, —C(O)—N(H)R^{14b}, —C(O)—N(H)OR^{14b}, —C(O)—NR^{14b}R^{15b} and —NR^{15b}C(O)R^{14b}; or

R^{12b} is selected from the group consisting of C₁-C₆ straight alkyl, C₃-C₆ branched alkyl, C₂-C₆ alkenyl, C₃-C₈ cyclic alkyl, hydroxy-C₁-C₆ straight alkyl, hydroxy-C₃-C₆ branched alkyl, hydroxy-C₂-C₆ alkenyl, hydroxy-C₃-C₈ cyclic alkyl, wherein all may be optionally substituted by one or more independently selected from the group consisting of halo, lower alkyl, cycloalkyl, acyl, hydroxy, hydroxyalkyl, heterocyclic, heteroaryl, amino, aminoalkyl, —NR^{4b}R^{15b}, oxo, cyano, carboxy, carboxyalkyl, alkoxycarbonyl, —OR^{16b}, —C(O)R^{16b}, —C(O)—NH₂, —C(O)—N(H)R^{14b}—C(O)—N(H)OR^{14b}, C(O)—NR^{14b}R^{15b} and —NR^{15b}C(O)R^{14b};

R^{13b} is selected from the group consisting of hydrogen, hydroxy, heterocyclic and heteroaryl, wherein all may be optionally substituted by one or more independently selected from the group consisting of halo, alkyl, lower alkyl, acyl, oxo, hydroxy, hydroxyalkyl, heterocyclic, heteroaryl, cyano, amino, aminoalkyl, and carboxy;

R^{14b} and R^{15b} are independently selected from the group consisting of hydrogen, C₁-C₆ straight alkyl, C₁-C₆ branched alkyl, heteroaryl, heterocycle, and acyl, wherein all may be optionally substituted by one or more independently selected from the group consisting of halo, alkyl, lower alkyl, alkenyl, cycloalkyl, acyl, hydroxy, hydroxyalkyl, heterocyclic, heteroaryl, aryl, amino, aminoalkyl, oxo, cyano, carboxy, carboxyalkyl, alkoxycarbonyl, and —OR^{16b}; and

R^{16b} is independently selected from the group consisting of C₁-C₄ straight alkyl, C₁-C₄ branched alkyl, heterocyclic and heteroaryl, wherein all may be substituted by one or more independently selected from the group consisting of halo, alkyl, lower alkyl, acyl, oxo, hydroxy, hydroxyalkyl, heterocyclic, heteroaryl, aryl, amino, aminoalkyl, cyano, and carboxy.

In another embodiment, the compound of Formula V is a compound wherein R^{12b} is selected from the group consisting of carboxy-C₁-C₄ straight alkyl, carboxy-C₃-C₆ branched alkyl, (C(O)NHR^{13b})—C₁-C₄ straight alkyl and (C(O)NHR^{13b})—C₃-C₆ branched alkyl, wherein all may be optionally substituted by one or more independently selected from the group consisting of halo, lower alkyl, acyl, hydroxy, hydroxyalkyl, heterocyclic, heteroaryl, amino, aminoalkyl, oxo, cyano, and alkoxycarbonyl; and

R^{13b} is selected from the group consisting of hydrogen, hydroxy, heterocyclic and heteroaryl, wherein all may be optionally substituted by one or more independently selected from the group consisting of halo, alkyl, lower alkyl, acyl, oxo, hydroxy, hydroxyalkyl, heterocyclic, heteroaryl, cyano, amino, aminoalkyl, and carboxy.

In another embodiment, the compound of Formula V is a compound wherein R^{12b} is selected from the group consisting of carboxy-C₁-C₄ straight alkyl and carboxy-C₃-C₆ branched alkyl, wherein all may be optionally substituted by one or more independently selected from the group consisting of halo, lower alkyl, acyl, hydroxy, hydroxyalkyl, heterocyclic, heteroaryl, and amino.

In another embodiment, the compound of Formula V is a compound wherein R^{12b} is selected from the group consisting of C₁-C₆ straight alkyl, C₃-C₆ branched alkyl, C₂-C₆ alkenyl, C₃-C₈ cyclic alkyl, hydroxy-C₁-C₆ straight alkyl, hydroxy-C₃-C₆ branched alkyl, hydroxy-C₂-C₆ alkenyl, hydroxy-C₃-C₈ cyclic alkyl, wherein all may be optionally substituted by one or more independently selected from the group consisting of halo, lower alkyl, acyl, hydroxy, hydroxyalkyl, heterocyclic, heteroaryl, amino, aminoalkyl, oxo, cyano, and alkoxycarbonyl; and

R^{13b} is selected from the group consisting of hydrogen, hydroxy, heterocyclic and heteroaryl, wherein all may be optionally substituted by one or more independently selected from the group consisting of halo, alkyl, lower alkyl, acyl, oxo, hydroxy, hydroxyalkyl, heterocyclic, heteroaryl, cyano, amino, aminoalkyl, and carboxy.

In another embodiment, the compound of Formula V is a compound wherein R^{12b} is selected from the group consisting of hydroxy-C₁-C₄ straight alkyl and hydroxy-C₃-C₆ branched alkyl, wherein all may be optionally substituted by one or more independently selected from the group consisting of halo, lower alkyl, acyl, hydroxy, hydroxyalkyl, heterocyclic, heteroaryl, and amino.

In another embodiment, the compound of Formula V is a compound wherein R^{12b} is selected from the group consisting of C₁-C₄ straight alkyl and C₃-C₆ branched alkyl, wherein all may be optionally substituted by one or more independently selected from the group consisting of halo, lower alkyl, acyl, hydroxy, hydroxyalkyl, heterocyclic, heteroaryl, and amino.

In one embodiment, compounds, pharmaceutical compositions and methods of treatment or prophylaxis of an inflammatory condition, and in particular asthma, comprising administering to a host in need thereof a compound of Formula VI, or a pharmaceutically acceptable salt, ester, prodrug or derivative thereof are provided:

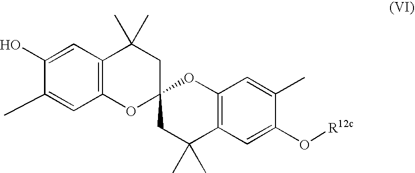

wherein:

R^{12c} is selected from the group consisting of carboxy-C₁-C₆ straight alkyl, carboxy-C₃-C₆ branched alkyl, carboxy-C₂-C₆ alkenyl, carboxy-C₃-C₈ cyclic alkyl, (C(O)NHR^{13c})—C₁-C₆ straight alkyl, (C(O)NHR^{13c})—C₃-C₆ branched alkyl, (C(O)NHR^{13c})—C₂-C₆ alkenyl, (C(O)NHR^{13c})—C₃-C₈ cyclic alkyl, heteroaralkyl, heterocyclicalkyl, and aralkyl, wherein all may be optionally substituted by one or more independently selected from the group consisting of halo, alkyl, lower alkyl, alkenyl, cycloalkyl, acyl, hydroxy, hydroxyalkyl, heterocyclic, heteroaryl, aryl, amino, aminoalkyl, —NR^{14c}R^{15c}, oxo, cyano, carboxy, carboxyalkyl, alkoxycarbonyl, —OR^{16c}, —C(O)R^{16c}, —C(O)—NH₂, —C(O)—N(H)R^{14c}, —C(O)—N(H)OR^{14c}, C(O)—NR^{14c}R^{15c}, NR^{15c}C(O)R^{14c}, —NR^{15c}C(O)NR^{14c}R^{15c}, —OC(O)NR^{14c}R^{15c}, —NR^{15c}C(O)OR^{16c}, —S(O)ₙ—R^{16c}, —S(O)₂—NH₂, —S(O)₂—N(H)R^{14c} and —S(O)₂—NR^{14c}R^{15c}; or

R^{12c} is selected from the group consisting of C₁-C₆ straight alkyl, C₃-C₆ branched alkyl, C₂-C₆ alkenyl, C₃-C₈ cyclic alkyl, hydroxy-C₁-C₆ straight alkyl, hydroxy-C₃-C₆ branched alkyl, hydroxy-C₂-C₆ alkenyl, hydroxy-C₃-C₈ cyclic alkyl, wherein all may be optionally substituted by one or more independently selected from the group consisting of halo, alkyl, lower alkyl, alkenyl, cycloalkyl, acyl, hydroxy, hydroxyalkyl, heterocyclic, heteroaryl, aryl, amino, aminoalkyl, —NR^{14c}R^{15c}, oxo, cyano, carboxy, carboxyalkyl, alkoxycarbonyl, —OR^{16c}, —C(O)R^{16c}, —C(O)—NH₂, —C(O)—N(H)R^{14c}, —C(O)—N(H)OR^{14c}, —C(O)—NR^{14c}R^{15c}, NR^{15c}C(O)R^{14c}, NR^{15c}C(O)NR^{14c}R^{15c}, —OC(O)NR^{14c}R^{15c}, —NR^{15c}C(O)OR^{16c}, —S(O)ₙ—R^{16c}, —S(O)₂—NH₂, —S(O)₂—N(H)R^{14c} and —S(O)₂—NR^{14c}R^{15c};

each n is independently 0, 1, or 2;

R^{13c} is selected from the group consisting of hydrogen, hydroxy, C₁-C₆ straight alkyl, C₁-C₆ branched alkyl, C₂-C₆ alkenyl, C₃-C₈ cyclic alkyl, heterocyclic, heteroaryl and aryl, wherein all may be optionally substituted by one or more independently selected from the group consisting of halo, alkyl, lower alkyl, acyl, oxo, hydroxy, hydroxyalkyl, heterocyclic, heteroaryl, cyano, amino, aminoalkyl, and carboxy;

R^{14c} and R^{15c} are selected from the group consisting of hydrogen, C₁-C₆ straight alkyl, C₁-C₆ branched alkyl, C₂-C₆ alkenyl, C₃-C₈ cyclic alkyl; aryl, heteroaryl, heterocycle, and acyl, wherein all may be optionally substituted by one or more independently selected from the group consisting of halo, alkyl, lower alkyl, alkenyl, cycloalkyl, acyl, hydroxy, hydroxyalkyl, heterocyclic, heteroaryl, aryl, amino, aminoalkyl, oxo, cyano, carboxy, carboxyalkyl, alkoxycarbonyl, and —OR^{16c};

R^{14c} and R^{15c} taken together may form a 4- to 12-membered monocyclic, bicyclic, tricyclic or benzofused ring;

R^{16c} is independently selected from the group consisting of C₁-C₆ straight alkyl, C₁-C₆ branched alkyl, C₂-C₆ alkenyl, C₃-C₈ cyclic alkyl; heterocyclic, heteroaryl and aryl, wherein all may be substituted by one or more independently selected from the group consisting of halo, alkyl, lower alkyl, acyl, oxo, hydroxy, hydroxyalkyl, heterocyclic, heteroaryl, aryl, amino, aminoalkyl, cyano, and carboxy.

In one embodiment, the compound of Formula VI is a compound wherein R^{12c} is selected from the group consisting of carboxy-C₁-C₆ straight alkyl, carboxy-C₃-C₆ branched alkyl, carboxy-C₃-C₈ cyclic alkyl, (C(O)NHR^{13c})—C₁-C₆ straight alkyl, (C(O)NHR^{13c})—C₃-C₆ branched alkyl, (C(O)NHR^{13c})—C₃-C₈ cyclic alkyl, heteroaralkyl, and heterocyclicalkyl, wherein all may be optionally substituted by one or more independently selected from the group consisting of halo, lower alkyl, cycloalkyl, acyl, hydroxy, hydroxyalkyl, heterocyclic, heteroaryl, amino, aminoalkyl, —NR^{14c}R^{15c}, oxo, cyano, carboxy, carboxyalkyl, alkoxycarbonyl, —OR^{16c}, —C(O)R^{16c}, —C(O)—NH₂, —C(O)—N(H)R^{14c}, —C(O)—N(H)OR^{14c}, —C(O)—NR^{14c}R^{15c} and NR^{15c}C(O)R^{14c}; or

R^{12c} is selected from the group consisting of C₁-C₆ straight alkyl, C₃-C₆ branched alkyl, C₂-C₆ alkenyl, C₃-C₈ cyclic alkyl, hydroxy-C₁-C₆ straight alkyl, hydroxy-C₃-C₆ branched alkyl, hydroxy-C₂-C₆ alkenyl, hydroxy-C₃-C₈ cyclic alkyl, wherein all may be optionally substituted by one or more independently selected from the group consisting of halo, lower alkyl, cycloalkyl, acyl, hydroxy, hydroxyalkyl, heterocyclic, heteroaryl, amino, aminoalkyl, —NR^{14c}R^{15c}, oxo, cyano, carboxy, carboxyalkyl, alkoxycarbonyl, OR^{16c}, —C(O)R^{16c}, —C(O)—NH₂, —C(O)—N(H)R^{14c}, —C(O)—N(H)OR^{14c}, C(O)—NR^{14c}R^{15c} and —NR^{15c}C(O)R^{14c};

R^{13c} is selected from the group consisting of hydrogen, hydroxy, heterocyclic and heteroaryl, wherein all may be optionally substituted by one or more independently selected from the group consisting of halo, alkyl, lower alkyl, acyl, oxo, hydroxy, hydroxyalkyl, heterocyclic, heteroaryl, cyano, amino, aminoalkyl, and carboxy;

R^{14c} and R^{15c} are independently selected from the group consisting of hydrogen, C₁-C₆ straight alkyl, C₁-C₆ branched alkyl, heteroaryl, heterocycle, and acyl, wherein all may be optionally substituted by one or more independently selected from the group consisting of halo, alkyl, lower alkyl, alkenyl, cycloalkyl, acyl, hydroxy, hydroxyalkyl, heterocyclic, heteroaryl, aryl, amino, aminoalkyl, oxo, cyano, carboxy, carboxyalkyl, alkoxycarbonyl, and —OR^{16b}; and

R^{16c} is independently selected from the group consisting of C₁-C₄ straight alkyl, C₁-C₄ branched alkyl, heterocyclic and heteroaryl, wherein all may be substituted by one or more independently selected from the group consisting of halo, alkyl, lower alkyl, acyl, oxo, hydroxy, hydroxyalkyl, heterocyclic, heteroaryl, aryl, amino, aminoalkyl, cyano, and carboxy.

In another embodiment, the compound of Formula VI is a compound wherein R^{12c} is selected from the group consisting of carboxy-C₁-C₄ straight alkyl, carboxy-C₃-C₆ branched alkyl, (C(O)NHR^{13c})—C₁-C₄ straight alkyl and (C(O)NHR^{13c})—C₃-C₆ branched alkyl, wherein all may be optionally substituted by one or more independently selected from the group consisting of halo, lower alkyl, acyl, hydroxy, hydroxyalkyl, heterocyclic, heteroaryl, amino, aminoalkyl, oxo, cyano, and alkoxycarbonyl; and

R^{13c} is selected from the group consisting of hydrogen, hydroxy, heterocyclic and heteroaryl, wherein all may be optionally substituted by one or more independently selected from the group consisting of halo, alkyl, lower alkyl, acyl, oxo, hydroxy, hydroxyalkyl, heterocyclic, heteroaryl, cyano, amino, aminoalkyl, and carboxy.

In another embodiment, the compound of Formula VI is a compound wherein R^{12c} is selected from the group consisting of carboxy-C₁-C₄ straight alkyl and carboxy-C₃-C₆ branched alkyl, wherein all may be optionally substituted by one or more independently selected from the group consisting of halo, lower alkyl, acyl, hydroxy, hydroxyalkyl, heterocyclic, heteroaryl, and amino.

In another embodiment, the compound of Formula VI is a compound wherein R^{12c} is selected from the group consisting of C₁-C₆ straight alkyl, C₃-C₆ branched alkyl, C₂-C₆ alkenyl, C₃-C₈ cyclic alkyl, hydroxy-C₁-C₆ straight alkyl, hydroxy-C₃-C₆ branched alkyl, hydroxy-C₂-C₆ alkenyl, hydroxy-C₃-C₈ cyclic alkyl, wherein all may be optionally substituted by one or more independently selected from the group consisting of halo, lower alkyl, acyl, hydroxy, hydroxyalkyl, heterocyclic, heteroaryl, amino, aminoalkyl, oxo, cyano, and alkoxycarbonyl; and

R^{13c} is selected from the group consisting of hydrogen, hydroxy, heterocyclic and heteroaryl, wherein all may be optionally substituted by one or more independently selected from the group consisting of halo, alkyl, lower alkyl, acyl, oxo, hydroxy, hydroxyalkyl, heterocyclic, heteroaryl, cyano, amino, aminoalkyl, and carboxy.

In another embodiment, the compound of Formula VI is a compound wherein R^{12c} is selected from the group consisting of hydroxy-C₁-C₄ straight alkyl and hydroxy-C₃-C₆ branched alkyl, wherein all may be optionally substituted by one or more independently selected from the group consisting of halo, lower alkyl, acyl, hydroxy, hydroxyalkyl, heterocyclic, heteroaryl, and amino.

In another embodiment, the compound of Formula VI is a compound wherein R^{12c} is selected from the group consisting of C₁-C₄ straight alkyl and C₃-C₆ branched alkyl, wherein all may be optionally substituted by one or more independently selected from the group consisting of halo, lower alkyl, acyl, hydroxy, hydroxyalkyl, heterocyclic, heteroaryl, and amino.

In one embodiment, a compound of any one of Formula II, III, IV, V or VI is a compound wherein R^{12*}, R^{12**}, R^{12a}, R^{12b} or R^{12c} is selected from the group consisting of carboxy-C₁-C₄ straight alkyl, carboxy-C₃-C₆ branched alkyl, (C(O)NHR¹³)—C₁-C₄ straight alkyl and (C(O)NHR¹³)—C₃-C₆ branched alkyl, wherein all may be optionally substituted by one or more independently selected from the group consisting of halo, lower alkyl, acyl, hydroxy, hydroxyalkyl, heterocyclic, heteroaryl, amino, aminoalkyl, oxo, cyano, and alkoxycarbonyl. In one subembodiment, the substituents are independently selected from lower alkyl, acyl, hydroxy, hydroxyalkyl and alkoxycarbonyl.

In one embodiment, a compound of any one of Formula II, III, IV, V or VI is a compound wherein R^{12*}, R^{12**}, R^{12a}, R^{12b} or R^{12c} is selected from the group consisting of C₁-C₆ straight alkyl, C₃-C₆ branched alkyl, C₂-C₆ alkenyl, C₃-C₈ cyclic alkyl, hydroxy-C₁-C₆ straight alkyl, hydroxy-C₃-C₆ branched alkyl, hydroxy-C₂-C₆ alkenyl, hydroxy-C₃-C₈ cyclic alkyl, wherein all may be optionally substituted by one or more independently selected from the group consisting of halo, alkyl, lower alkyl, alkenyl, cycloalkyl, acyl, hydroxy, hydroxyalkyl, heterocyclic, heteroaryl, aryl, amino, aminoalkyl, oxo, cyano, carboxy, carboxyalkyl, and alkoxycarbonyl.

In particular embodiments, R¹², R^{12*}, R^{12**}, R^{12a}, R^{12b} or R^{12c} is selected from the group consisting of hydroxy-C₁-C₆ straight alkyl, hydroxy-C₃-C₆ branched alkyl, hydroxy-C₂-C₆ alkenyl, hydroxy-C₃-C₈ cyclic alkyl, wherein all may be optionally substituted by one or more independently selected from the group consisting of halo, alkyl, lower alkyl, alkenyl, cycloalkyl, acyl, hydroxy, hydroxyalkyl, heterocyclic, heteroaryl, aryl, amino, aminoalkyl, oxo, cyano, carboxy, carboxyalkyl, and alkoxycarbonyl.

In certain embodiments, R¹², R^{12*}, R^{12**}, R^{12a}, R^{12b} or R^{12c} is C₁-C₄ branched alkyl, optionally substituted by one or more hydroxy, cyano and heteroaryl. In particular embodiments, R¹², R^{12*}, R^{12**}, R^{12a}, R^{12b} or R^{12c} is selected from the group consisting of hydrogen, cyanomethyl, tetrazolylmethyl, imidazolylethyl, hydroxymethyl, 2-methyl-2-hydroxypropyl, and hydroxyethyl. In more particular embodiments, R¹², R^{12*}, R^{12**}, R^{12a}, R^{12b} or R^{12c} is selected from the group consisting of hydrogen, hydroxymethyl, 2-methyl-2-hydroxypropyl, and hydroxyethyl.

In certain embodiments, R¹², R^{12*}, R^{12**}, R^{12a}, R^{12b} or R^{12c} is unsubstituted heteroaryl. In other embodiments, R¹², R^{12*}, R^{12**}, R^{12a}, R^{12b} or R^{12c} is heteroaryl, substituted with one substituent. In yet other embodiments, R¹², R^{12*}, R^{12**}, R^{12a}, R^{12b} or R^{12c} is heteroaryl substituted with more than one substituent. In particular embodiments, substituents on R¹², R^{12*}, R^{12**}, R^{12a}, R^{12b} or R^{12c} are selected from alkyl, hydroxyalkyl, carboxy and carboxyalkyl. In more particular embodiments, R¹², R^{12*}, R^{12**}, R^{12a}, R^{12b} or R^{12c} is selected from the group consisting of furyl, thienyl, thiazolyl, pyrazolyl, pyrrolyl, triazolyl, tetrazolyl, oxazolyl, imidazolyl, isooxazolyl, isothiazolyl, pyridyl, pyridazinyl, pyrimidinyl, pyrazinyl, thiazinyl, benzimidazolyl, tetrahydrobenzimidazolyl, benzofuryl, benzothienyl, benzothiazolyl, benzopyrazolyl, indolyl, benzotriazolyl, benzoxazolyl, benzoisoxazolyl, benzoisothiazolyl, benzopyridyl, benzopyridazinyl, benzopyrimidinyl, benzopyrazinyl, and benzothiazinyl, wherein all may be substituted by one or more independently selected from the group consisting of fluoro, chloro, bromo, iodo, trifluoromethyl, methyl, ethyl, iso-propyl, tert-butyl, hydroxymethyl, 2-methyl-2-hydroxyethyl, amino, and carboxy. In more particular embodiments, R¹², R^{12*}, R^{12**}, R^{12a}, R^{12b} or R^{12c} is selected from the group consisting of furyl, thienyl, thiazolyl, pyrazolyl, pyrrolyl and imidazolyl, wherein all may be substituted by one or more independently selected from the group consisting of fluoro, chloro, trifluoromethyl, methyl, hydroxymethyl, 2-methyl-2-hydroxyethyl, amino, and carboxy. In even more particular embodiments, R¹², R^{12*}, R^{12**}, R^{12a}, R^{12b} or R^{12c} is pyrrolyl or imidazolyl, wherein all may be substituted by one or more independently selected from the group consisting of methyl, hydroxymethyl, 2-methyl-2-hydroxyethyl, and carboxy.

In one embodiment the compound is

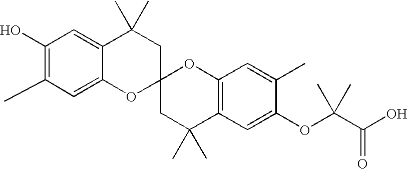

or an enantiomer thereof, or a pharmaceutically acceptable salt, ester, prodrug or derivative thereof.

In one embodiment the compound is

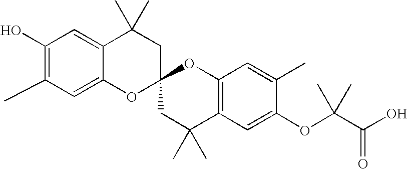

or a pharmaceutically acceptable salt, ester, prodrug or derivative thereof.

In one embodiment the compound is

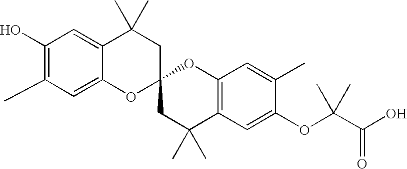

or a pharmaceutically acceptable salt, ester, prodrug or derivative thereof.

In a subembodiment, the pharmaceutical compositions and methods of treatment or prophylaxis of an inflammatory condition, and in particular asthma, comprises administering to a host in need thereof a compound

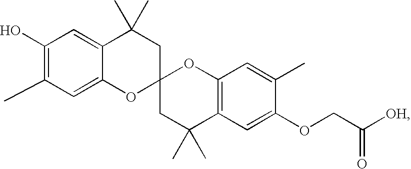

or a pharmaceutically acceptable salt, ester, prodrug or derivative thereof.

In one embodiment the compound is

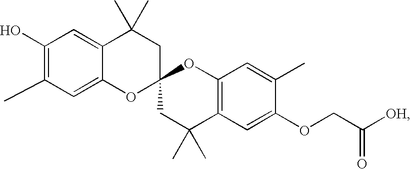

or a pharmaceutically acceptable salt, ester, prodrug or derivative thereof.

In another embodiment the compound is

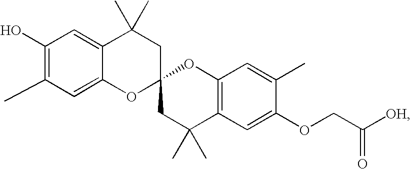

or a pharmaceutically acceptable salt, ester, prodrug or derivative thereof.

In one embodiment, the pharmaceutical compositions and methods of treatment or prophylaxis of an inflammatory condition, and in particular asthma, comprises administering to a host in need thereof a compound

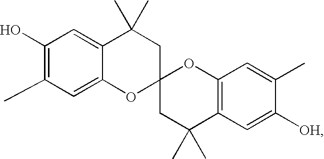

or a pharmaceutically acceptable salt, ester, prodrug or derivative thereof.

In another embodiment, the pharmaceutical compositions and methods of treatment or prophylaxis of an inflammatory condition, and in particular asthma, comprises administering to a host in need thereof a compound

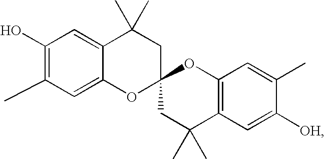

or a pharmaceutically acceptable salt, ester, prodrug or derivative thereof.

In another embodiment, the pharmaceutical compositions and methods of treatment or prophylaxis of an inflammatory condition, and in particular asthma, comprises administering to a host in need thereof a compound

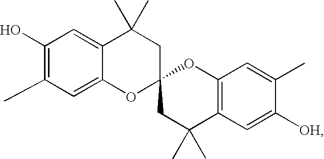

or a pharmaceutically acceptable salt, ester, prodrug or derivative thereof.

In some embodiments, compounds of the invention are as defined below in Table A:

TABLE A

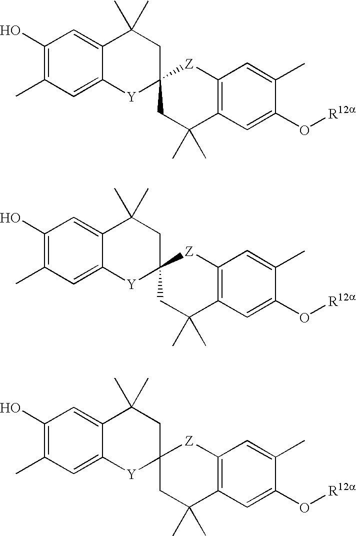

| Y and Z are each | R^{12α} |
|---|---|
| O | 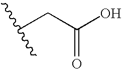 |
| O | 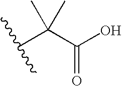 |
| O | 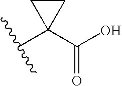 |
| O | 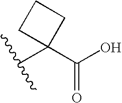 |
| O | 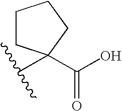 |
| O | 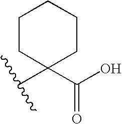 |
| O | 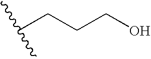 |
| O | 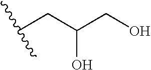 |
| O | 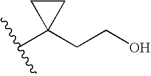 |
| O | 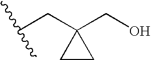 |
| O | 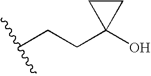 |
| O | 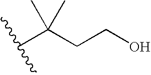 |
| O | 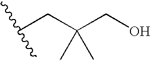 |
| O | 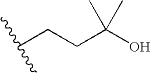 |
| O | 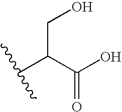 |
| O | 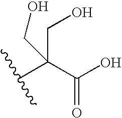 |
| O | 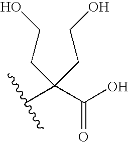 |
| O | 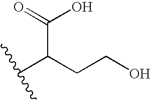 |
| O | 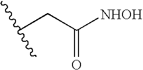 |
| O | 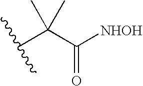 |
| O | 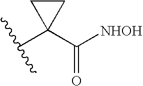 |
| O | 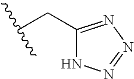 |
| O | 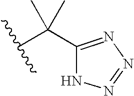 |
| O | 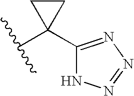 |
| O | 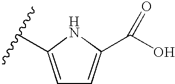 |
| O | 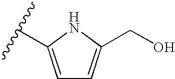 |
| O | 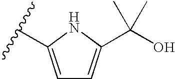 |
| O | 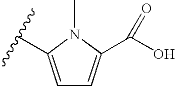 |
| O | 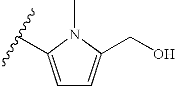 |
| O | 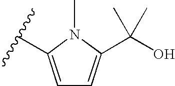 |
| O | 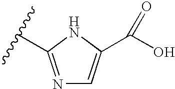 |
| O | 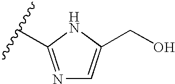 |
| O | 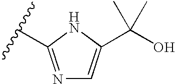 |
| O | 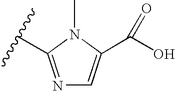 |
| O | 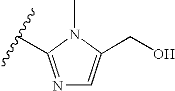 |
| O | 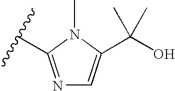 |
| O | 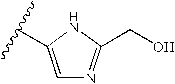 |
| O | 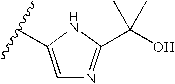 |
| O | 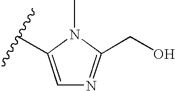 |
| O | 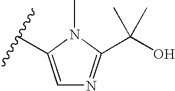 |
| O | 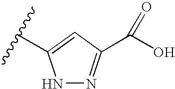 |
| O | 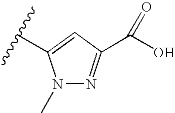 |
| O | 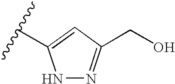 |
| O | 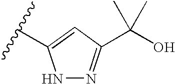 |
| O | 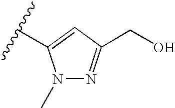 |
| O | 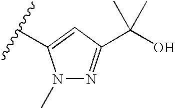 |
| O | 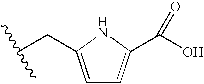 |
| O | 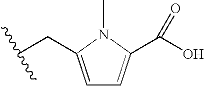 |
| O | 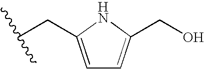 |
| O | 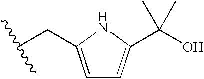 |
| O | 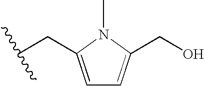 |
| O | 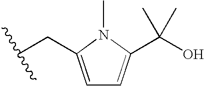 |
| O | 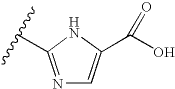 |
| O | 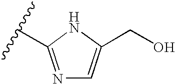 |
| O | 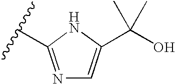 |
| O | 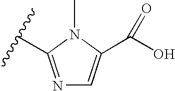 |
| O | 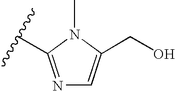 |
| O | 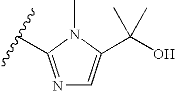 |
| O | 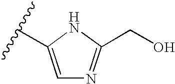 |
| O | 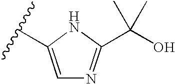 |
| O | 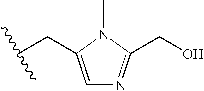 |
| O | 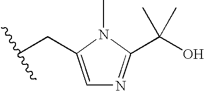 |
| O | 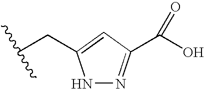 |
| O | 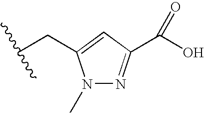 |
| O | 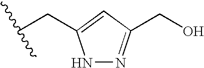 |
| O | 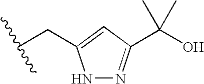 |
| O | 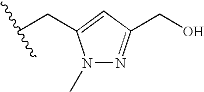 |
| O | 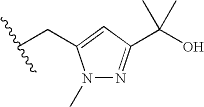 |
| O | 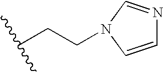 |
| O | 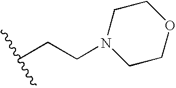 |
| O | 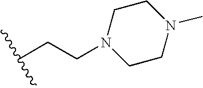 |
| O | 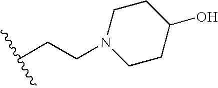 |
| O | 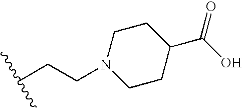 |

In other embodiments, compounds of the invention are as defined below in Table B:

TABLE B

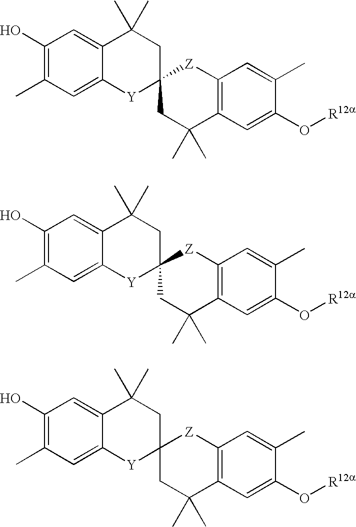

| Y and Z are each | R^{12α} |
|---|---|
| S | 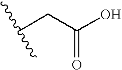 |
| S | 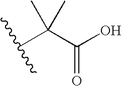 |
| S | 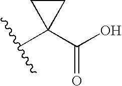 |
| S | 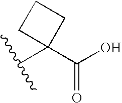 |
| S | 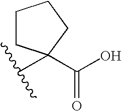 |
| S | 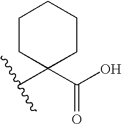 |
| S | 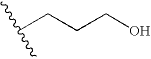 |
| S | 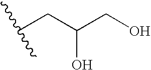 |
| S | 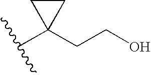 |
| S | 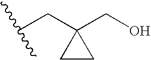 |
| S | 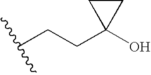 |
| S | 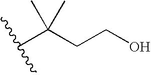 |
| S | 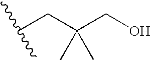 |
| S | 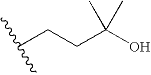 |
| S | 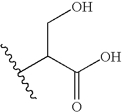 |
| S | 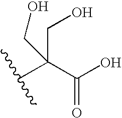 |
| S | 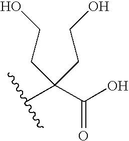 |
| S | 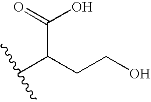 |
| S | 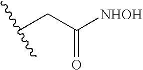 |
| S | 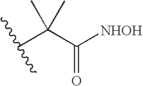 |
| S | 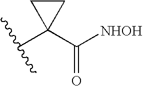 |
| S | 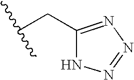 |
| S | 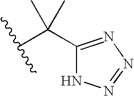 |
| S | 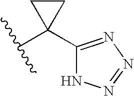 |
| S | 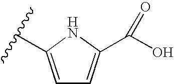 |
| S | 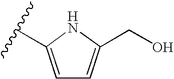 |
| S | 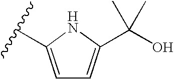 |
| S | 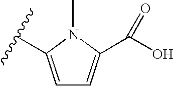 |
| S | 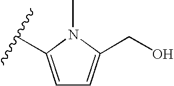 |
| S | 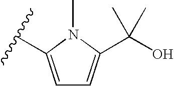 |
| S | 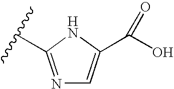 |
| S | 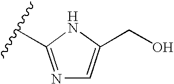 |
| S | 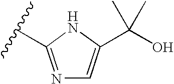 |
| S | 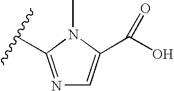 |
| S | 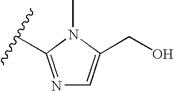 |
| S | 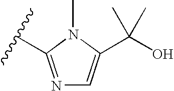 |
| S | 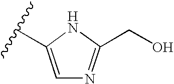 |
| S | 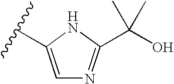 |
| S | 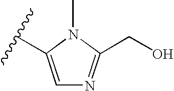 |
| S | 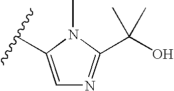 |
| S | 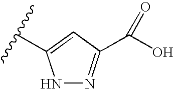 |
| S | 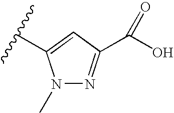 |
| S | 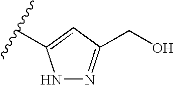 |
| S | 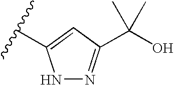 |
| S | 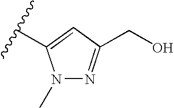 |
| S | 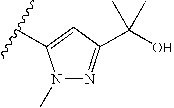 |
| S | 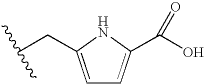 |
| S | 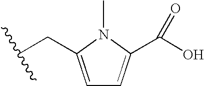 |
| S | 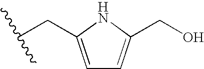 |
| S | 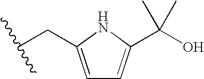 |
| S | 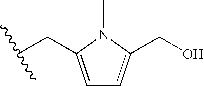 |
| S | 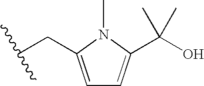 |
| S | 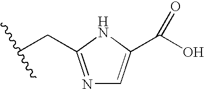 |
| S | 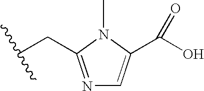 |
| S | 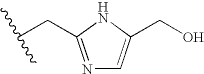 |
| S | 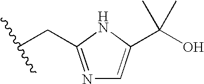 |
| S | 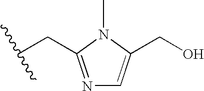 |
| S | 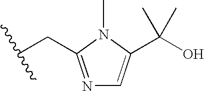 |
| S | 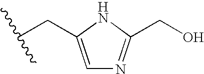 |
| S | 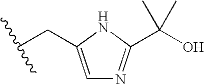 |
| S | 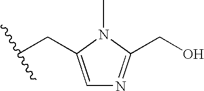 |
| S | 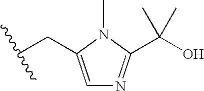 |
| S | 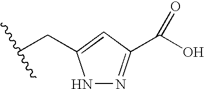 |
| S | 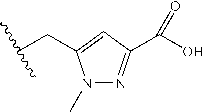 |
| S | 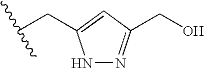 |
| S | 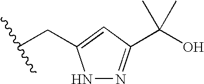 |
| S | 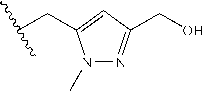 |
| S | 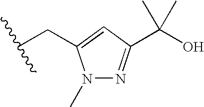 |
| S | 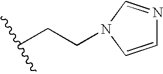 |
| S | 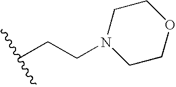 |
| S | 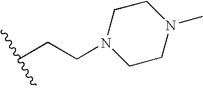 |
| S | 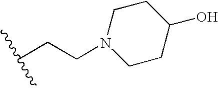 |
| S | 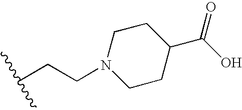 |

In other embodiments, compounds of the invention are as defined below in Table C:

TABLE C

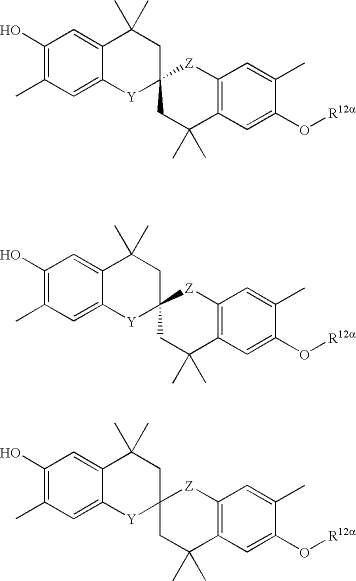

| Y and Z are each | R^{12α} |
|---|---|
| Se | 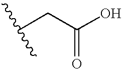 |
| Se | 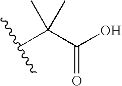 |
| Se | 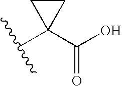 |
| Se | 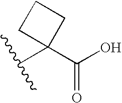 |
| Se | 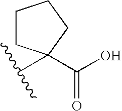 |
| Se | 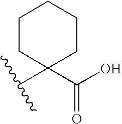 |
| Se | 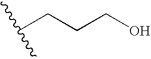 |
| Se | 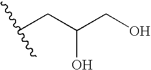 |
| Se | 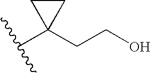 |
| Se | 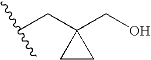 |
| Se | 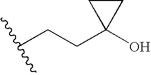 |
| Se | 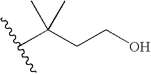 |
| Se | 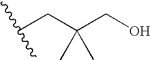 |
| Se | 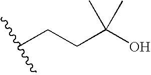 |
| Se | 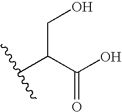 |
| Se | 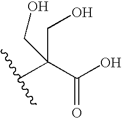 |
| Se | 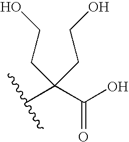 |
| Se | 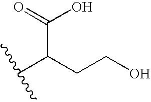 |
| Se | 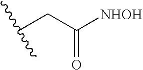 |
| Se | 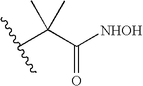 |
| Se | 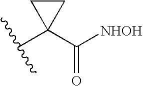 |
| Se | 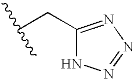 |
| Se | 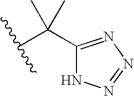 |
| Se | 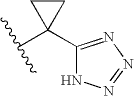 |
| Se | 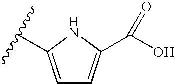 |
| Se | 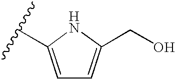 |
| Se | 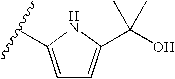 |
| Se | 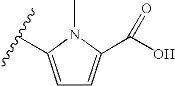 |
| Se | 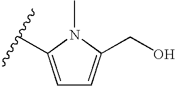 |
| Se | 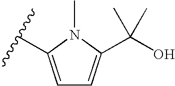 |
| Se | 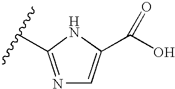 |
| Se | 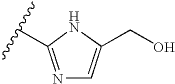 |
| Se | 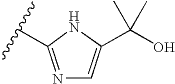 |
| Se | 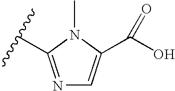 |
| Se | 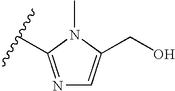 |
| Se | 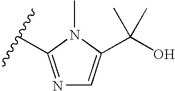 |
| Se | 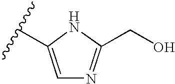 |
| Se | 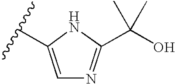 |
| Se | 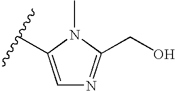 |
| Se | 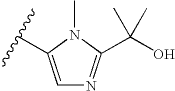 |
| Se | 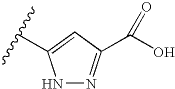 |
| Se | 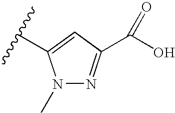 |
| Se | 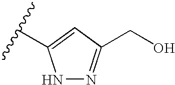 |
| Se | 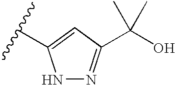 |
| Se | 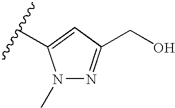 |
| Se | 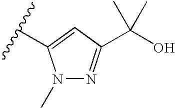 |
| Se | 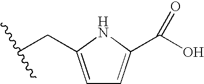 |
| Se | 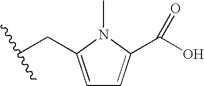 |
| Se | 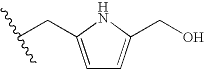 |
| Se | 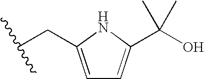 |
| Se | 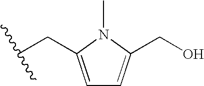 |
| Se | 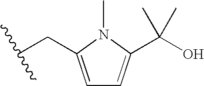 |
| Se | 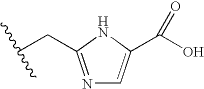 |
| Se | 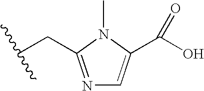 |
| Se | 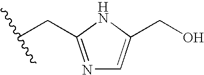 |
| Se | 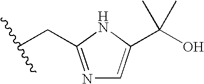 |
| Se | 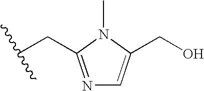 |
| Se | 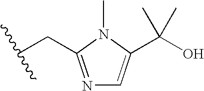 |
| Se | 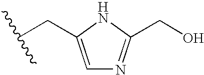 |
| Se | 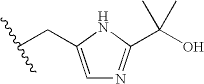 |
| Se | 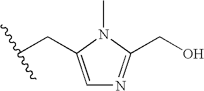 |
| Se | 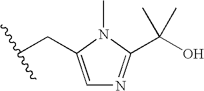 |
| Se | 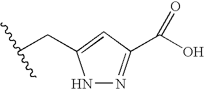 |
| Se | 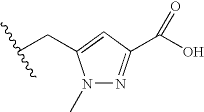 |
| Se | 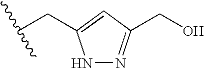 |
| Se | 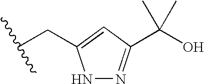 |
| Se | 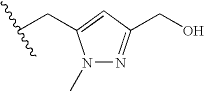 |
| Se | 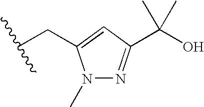 |
| Se | 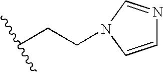 |
| Se | 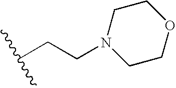 |
| Se | 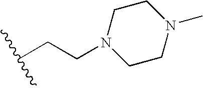 |
| Se | 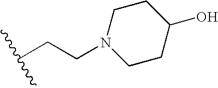 |
| Se | 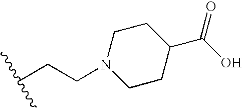 |

In certain embodiments, the compounds are present as enantiomers. In particular embodiments, the compound is present as a racemic mixture. The enantiomer can be named by the configuration at the chiral center, such as R or S. In certain embodiments, the compound is present as a racemic mixture of R- and S- enantiomers. In certain embodiments, the compound is present as a mixture of two enantiomers. In one embodiment, the mixture has an enantiomeric excess in R. In one embodiment, the mixture has an enantiomeric excess in S. In certain other embodiments, the compound is in an enantiomeric excess of the R- or S- enantiomer. The enantiomeric excess can be 51% or more, such as 51% or more, 55% or more, 60% or more, 65% or more, 70% or more, 75% or more, 80% or more, 85% or more, 90% or more, 95% or more, 98% or more, or 99% or more in the single enantiomer. The enantiomeric excess can be 51% or more, such as 51% or more, 55% or more, 60% or more, 65% or more, 70% or more, 75% or more, 80% or more, 85% or more, 90% or more, 95% or more, 98% or more, or 99% or more in the R enantiomer. The enantiomeric excess can be 51% or more, such as 51% or more, 55% or more, 60% or more, 65% or more, 70% or more, 75% or more, 80% or more, 85% or more, 90% or more, 95% or more, 98% or more, or 99% or more in the S enantiomer.

In other embodiments, the compound is substantially in the form of a single enantiomer. In some embodiments, the compound is present substantially in the form of the R enantiomer. In some embodiments, the compound is present substantially in the form of the S enantiomer. The phrase “substantially in the form of a single enantiomer” is intended to mean at least 70% or more in the form of a single enantiomer, for example 70% or more, 75% or more, 80% or more, 85% or more, 90% or more, 95% or more, 98% or more, or 99% or more in either the R or S enantiomer.

The enantiomer can be named by the direction in which it rotates the plane of polarized light. If it rotates the light clockwise as seen by the viewer towards whom the light is traveling, the isomer can be labeled (+) and if it rotates the light counterclockwise, the isomer can be labeled (−). In certain embodiments, the compound is present as a racemic mixture of (+) and (−) isomers. In certain embodiments, the compound is present as a mixture of two isomers. In one embodiment, the mixture has an excess in (+). In one embodiment, the mixture has an excess in (−). In certain other embodiments, the compound is in an excess of the (+) or (−) isomer. The isomeric excess can be 51% or more, such as 51% or more, 55% or more, 60% or more, 65% or more, 70% or more, 75% or more, 80% or more, 85% or more, 90% or more, 95% or more, 98% or more, or 99% or more in the (+) isomer. The enantiomeric excess can be 51% or more, such as 51% or more, 55% or more, 60% or more, 65% or more, 70% or more, 75% or more, 80% or more, 85% or more, 90% or more, 95% or more, 98% or more, or 99% or more in the (−) isomer.

In other embodiments, the compound is substantially in the form of a single optical isomer. In some embodiments, the compound is present substantially in the form of the (+) isomer. In other embodiments, the compound is present substantially in the form of the (−) isomer. The phrase “substantially in the form of a single optical isomer” is intended to mean at least 70% or more in the form of a single isomer, for example 70% or more, 75% or more, 80% or more, 85% or more, 90% or more, 95% or more, 98% or more, or 99% or more of either the (+) or (−) isomer.

### DEFINITIONS

Whenever a term in the specification is identified as a range (i.e. C₁₋₄ alkyl), the range independently refers to each element of the range. As a non-limiting example, C₁₋₄ alkyl means, independently, C₁, C₂, C₃ or C₄ alkyl. Similarly, when one or more substituents are referred to as being “independently selected from” a group, this means that each substituent can be any element of that group, and any combination of these groups can be separated from the group. For example, if R¹ and R² can be independently selected from X, Y and Z, this separately includes the groups R¹ is X and R² is X; R¹ is X and R² is Y; R¹ is X and R² is Z; R¹ is Y and R² is X; R¹ is Y and R² is Y; R¹ is Y and R² is Z; R¹ is Z and R² is X; R¹ is Z and R² is Y; and R¹ is Z and R² is Z.

The term “alkyl” is used herein, unless otherwise specified, refers to a saturated straight, branched, or cyclic (also identified as cycloalkyl), primary, secondary, or tertiary hydrocarbon, including but not limited to those of C₁ to C₆. Illustrative examples of alkyl groups are methyl, ethyl, propyl, isopropyl, cyclopropyl, butyl, secbutyl, isobutyl, tertbutyl, cyclobutyl, 1-methylbutyl, 1,1-dimethylpropyl, pentyl, cyclopentyl, isopentyl, neopentyl, cyclopentyl, hexyl, isohexyl, and cyclohexyl. Unless otherwise specified, the alkyl group can be unsubstituted or substituted with one or more moieties selected from the group consisting of alkyl, halo, haloalkyl, hydroxyl, carboxyl, acyl, acyloxy, amino, amido, carboxyl derivatives, alkylamino, dialkylamino, arylamino, alkoxy, aryloxy, nitro, cyano, thio, sulfonyl, ester, carboxylic acid, amide, phosphonyl, phosphinyl, thioether, oxime, or any other viable functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1991. In certain embodiments, alkyl may be optionally substituted by one or more halo, hydroxy, heterocyclic, heteroaryl, carboxy, —NRR′, alkoxycarbonyl, —NRC(O)R′, —NRC(O)NRR′, —NRC(O)OR′, —OC(O)NRR′, —OR′, —C(O)R′, —S(O)ₙ—R, —C(O)—NRR′, and/or cyano. In certain embodiments, the alkyl may be optionally substituted by one or more halo, alkyl, lower alkyl, alkenyl, cycloalkyl, acyl, hydroxy, hydroxyalkyl, heterocyclic, heteroaryl, aryl, amino, aminoalkyl, —NRR′, oxo, cyano, carboxy, carboxyalkyl, alkoxycarbonyl,, —OR′, —C(O)R, —C(O)—NH₂, —C(O)—N(H)R, —C(O)—NRR′, —NRC(O)R′, —NRC(O)OR′, —S(O)ₙ—R, —S(O)₂—NH₂, —S(O)₂—N(H)R and/or —S(O)₂—NRR′, wherein R and R′ are each independently selected from an alkyl, aryl, alkaryl or aralkyl group, or substituted alkyl, aryl, aralkyl or alkaryl, and n is 0, 1 or 2.

The term “lower alkyl,” unless otherwise specified, refers to a C₁ to C₅ saturated or unsaturated straight, branched carbon chain such as methyl, ethyl, isopropyl, n-butyl, tert-butyl, n-pentyl, sec-pentyl, 3-methylpentyl, and the like, or if appropriate, a cyclic (for example, cyclopropyl) alkyl group.

The term “halo” or “halogen,” refers to chloro, bromo, iodo, or fluoro.

The term “heteroaryl” or “heteroaromatic,” refers to an aromatic that includes at least one sulfur, oxygen, nitrogen or phosphorus in the aromatic ring. The term “heterocyclic” refers to a non-aromatic cyclic group wherein there is at least one heteroatom, such as oxygen, sulfur, nitrogen, or phosphorus in the ring. Nonlimiting examples of heteroaryl and heterocyclic groups include furyl, furanyl, pyridyl, pyrimidyl, thienyl, isothiazolyl, imidazolyl, tetrazolyl, pyrazinyl, benzofuranyl, benzothiophenyl, quinolyl, isoquinolyl, benzothienyl, isobenzofuryl, pyrazolyl, indolyl, isoindolyl, benzimidazolyl, purinyl, carbazolyl, oxazolyl, thiazolyl, isothiazolyl, 1,2,4-thiadiazolyl, isooxazolyl, pyrrolyl, quinazolinyl, cinnolinyl, phthalazinyl, xanthinyl, hypoxanthinyl, thiophene, furan, pyrrole, isopyrrole, pyrazole, imidazole, 1,2,3-triazole, 1,2,4-triazole, oxazole, isoxazole, thiazole, isothiazole, pyrimidine or pyridazine, pteridinyl, aziridines, thiazole, isothiazole, oxadiazole, thiazine, pyridine, pyrazine, piperazine, piperidine, pyrrolidine, oxaziranes, phenazine, phenothiazine, morpholinyl, pyrazolyl, pyridazinyl, pyrazinyl, quinoxalinyl, xanthinyl, hypoxanthinyl, pteridinyl, 5-azacytidinyl, 5-azauracilyl, triazolopyridinyl, imidazolopyridinyl, pyrrolopyrimidinyl, pyrazolopyrimidinyl, adenine, N⁶-alkylpurines, N⁶-benzylpurine, N⁶-halopurine, N⁶-vinypurine, N⁶-acetylenic purine, N⁶-acyl purine, N⁶-hydroxyalkyl purine, N6-thioalkyl purine, thymine, cytosine, 6-azapyrimidine, 2-mercaptopyrmidine, uracil, N⁵-alkylpyrimidines, N⁵-benzylpyrimidines, N⁵-halopyrimidines, N⁵-vinylpyrimidine, N⁵-acetylenic pyrimidine, N⁵-acyl pyrimidine, N⁵-hydroxyalkyl purine, and N⁶-thioalkyl purine, and isoxazolyl. The heteroaromatic or heterocyclic group can be optionally substituted with one or more substituent selected from halogen, haloalkyl, alkyl, alkoxy, hydroxy, carboxyl derivatives, amido, amino, alkylamino, dialkylamino. The heteroaromatic can be partially or totally hydrogenated as desired. Nonlimiting examples include dihydropyridine and tetrahydrobenzimidazole. In some embodiment, the heteroaryl may be optionally substituted by one or more halo, alkyl, lower alkyl, alkenyl, cycloalkyl, acyl, hydroxy, hydroxyalkyl, heterocyclic, heteroaryl, aryl, amino, aminoalkyl, —NRR′, oxo, cyano, carboxy, carboxyalkyl, alkoxycarbonyl,, —OR, —C(O)R, —C(O)—NH₂, —C(O)—N(H)R, —C(O)—NRR′, —NRC(O)R′, —NRC(O)OR′, —S(O)ₙ—R, —S(O)₂—NH₂, —S(O)₂—N(H)R and/or —S(O)₂—NRR′, wherein R and R′ are each independently selected from an alkyl, aryl, alkaryl or aralkyl group, or substituted alkyl, aryl, aralkyl or alkaryl, and n is 0, 1 or 2. Functional oxygen and nitrogen groups on the heteroaryl group can be protected as necessary or desired. Suitable protecting groups are well known to those skilled in the art, and include trimethylsilyl, dimethylhexylsilyl, t-butyldimethylsilyl, and t-butyldiphenylsilyl, trityl or substituted trityl, alkyl groups, acyl groups such as acetyl and propionyl, methanesulfonyl, and p-toluenelsulfonyl.

The term “aryl,” unless otherwise specified, refers to a carbon based aromatic ring, including phenyl, biphenyl, or naphthyl. The aryl group can be optionally substituted with one or more moieties selected from the group consisting of hydroxyl, acyl, amino, halo, alkylamino, alkoxy, aryloxy, nitro, cyano, sulfonic acid, sulfate, phosphonic acid, phosphate, or phosphonate, either unprotected, or protected as necessary, as known to those skilled in the art, for example, as taught in Greene, et al., Protective Groups in Organic Synthesis, John Wiley and Sons, Second Edition, 1991. In certain embodiments, the aryl group is optionally substituted by one or more halo, alkyl, lower alkyl, alkenyl, cycloalkyl, acyl, hydroxy, hydroxyalkyl, heterocyclic, heteroaryl, aryl, amino, aminoalkyl, —NRR′, oxo, cyano, carboxy, carboxyalkyl, alkoxycarbonyl, —OR, —C(O)R, —C(O)—NH₂, —C(O)—N(H)R, —C(O)—NRR′, —NRC(O)R′, —NRC(O)OR′, —S(O)ₙ—R, —S(O)₂—NH₂, —S(O)₂—N(H)R and/or —S(O)₂—NRR′, wherein R and R′ are each independently selected from an alkyl, aryl, alkaryl or aralkyl group, or substituted alkyl, aryl, aralkyl or alkaryl, and n is 0, 1 or 2.

The term “aralkyl,” unless otherwise specified, refers to an aryl group as defined above linked to the molecule through an alkyl group as defined above. The term “alkaryl,” unless otherwise specified, refers to an alkyl group as defined above linked to the molecule through an aryl group as defined above. Other groups, such as acyloxyalkyl, alkoxyalkyl, alkoxycarbonyl, alkoxycarbonylalkyl, alkylaminoalkyl, alkylthioalkyl, amidoalkyl, aminoalkyl, carboxyalkyl, dialkylaminoalkyl, haloalkyl, heteroaralkyl, heterocyclicalkyl, hydroxyalkyl, sulfonamidoalkyl, sulfonylalkyl and thioalkyl are named in a similar manner.

The term “alkoxy,” unless otherwise specified, refers to a moiety of the structure —O-alkyl, wherein alkyl is as defined above.

The term “acyl,” refers to a group of the formula —C(O)R wherein R is an alkyl, aryl, alkaryl or aralkyl group, or substituted alkyl, aryl, aralkyl or alkaryl.

The term “alkenyl” The term “alkenyl” means a monovalent, unbranched or branched hydrocarbon chain having one or more double bonds therein. The double bond of an alkenyl group can be unconjugated or conjugated to another unsaturated group. Suitable alkenyl groups include, but are not limited to (C₂-C₈)alkenyl groups, such as vinyl, alkyl, butenyl, pentenyl, hexenyl, butadienyl, pentadienyl, hexadienyl, 2-ethylhexenyl, 2-propyl-2-butenyl,4-(2-methyl-3-butene)-pentenyl. An alkenyl group can be unsubstituted or substituted with one or two suitable substituents.

The term “carbonyl” refers to a functional group composed of a carbon atom double-bonded to an oxygen atom: —C═O.

The term “amino” indicates presence of —NH₂.

The term “thio” indicates the presence of a sulfur group. The prefix thio- denotes that there is at least one extra sulfur atom added to the chemical. The prefix ‘thio-’ can also be placed before the name of a compound to mean that an oxygen atom in the compound has been replaced by a sulfur atom. Although typically the term “thiol” is used to indicate the presence of —SH, in instances in which the sulfur atom would be have improper valance a radical if the hydrogen is improperly designated, the terms ‘thio’ and ‘thiol’ are used interchangeably, unless otherwise indicated.

The term “amido” indicates a group —NH—C(O)—R.

The term “carboxy” designates the terminal group —C(O)OH.

The term “sulfonyl” indicates a group —S(═O)₂—R.

The term “pharmaceutically acceptable salt” refers to salts or complexes that retain the desired biological activity of the compounds of the present invention and exhibit minimal undesired toxicological effects. Nonlimiting examples of such salts are (a) acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalcturonic acid; (b) base addition salts formed with metal cations such as zinc, calcium, bismuth, barium, magnesium, aluminum, copper, cobalt, nickel, cadmium, sodium, potassium, and the like, or with a cation formed from ammonia, N,N-dibenzylethylenediamine, D-glucosamine, tetraethylammonium, or ethylenediamine; or (c) combinations of (a) and (b); e.g., a zinc tannate salt or the like. Also included in this definition are pharmaceutically acceptable quaternary salts known by those skilled in the art, which specifically include the quaternary ammonium salt of the formula —NR⁺A⁻, wherein R is as defined above and A is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

The term “protected” as used herein and unless otherwise defined refers to a group that is added to an oxygen, nitrogen, or phosphorus atom to prevent its further reaction or for other purposes. A wide variety of oxygen and nitrogen protecting groups are known to those skilled in the art of organic synthesis.

It should be understood that the various possible stereoisomers of the groups mentioned above and herein are within the meaning of the individual terms and examples, unless otherwise specified. As an illustrative example, “1-methyl-butyl” exists in both (R) and the (S) form, thus, both (R)-1-methyl-butyl and (S)-1-methyl-butyl is covered by the term “1-methyl-butyl”, unless otherwise specified.

The term “stereoisomers” refers to isomeric molecules whose atomic connectivity is the same but whose atomic arrangement in space is different.

The term “enantiomers” refers to compounds that are stereoisomers that are nonsuperimposable complete mirror images of each other. Enantiomers have, when present in a symmetric environment, identical chemical and physical properties except for their ability to rotate plane-polarized light by equal amounts but in opposite directions.

The term “racemic” refers to a mixture of equal parts of an optically active isomer and its enantiomer.

The term “diastereomers” refers to a pair stereoisomers that are not mirror images of each other and one or more stereogenic centers differ between the two stereoisomers, or one or more chiral centers have opposite configurations between the two stereoisomers.

### Methods of Use

The compounds of the invention can generally be administered to a host at risk of, or suffering from, an inflammatory condition. In one embodiment, the compounds are administered for the treatment or prophylaxis of an inflammatory disorder. In certain embodiments, the inflammatory disorder is a respiratory disorder. In particular embodiments, the inflammatory disorder is asthma or COPD. In other, separate embodiments the inflammatory disorder is a cardiovascular disorder. In certain embodiments, the inflammatory condition is mediated by known cytokines such as IL-6 or IL-8. In other embodiments, the inflammatory condition is unrelated to levels of any particular cytokines, such as IL-6 or IL-8. Certain of the compounds of the invention are useful in the treatment of inflammatory respiratory conditions, such as asthma, independently of their effect on inflammatory cytokines related to chemotaxis or antibody-mediated immune responses.

Cytokines are small secreted proteins which mediate and regulate immunity, inflammation, and hematopoiesis. They must be produced de novo in response to an immune stimulus. They generally (although not always) act over short distances and short time spans and at very low concentration. They act by binding to specific membrane receptors, which then signal the cell via second messengers, often tyrosine kinases, to alter its behavior (gene expression). Responses to cytokines include increasing or decreasing expression of membrane proteins (including cytokine receptors), proliferation, and secretion of effector molecules.

It is common for different cell types to secrete the same cytokine or for a single cytokine to act on several different cell types Cytokines are redundant in their activity, meaning similar functions can be stimulated by different cytokines. The largest group of cytokines stimulates immune cell proliferation and differentiation. This group includes Interleukin 1 (IL-1), which activates T cells; IL-2, which stimulates proliferation of antigen-activated T and B cells; IL-4, IL-5, and IL-6, which stimulate proliferation and differentiation of B cells; Interferon gamma (IFNγ), which activates macrophages; and IL-3, IL-7 and Granulocyte Monocyte Colony-Stimulating Factor (GM-CSF), which stimulate hematopoiesis. IL-6 is generally produced by monocytes, macrophages, Th2 cells and stromal cells. It acts on activated B cells to differentiate into plasma cells, plasma cells to induce antibody secretion, stem cells to induce differentiation, and on various other cells to induce acute inflammatory responses. IL-8, produced by macrophages and endothelial cells generally acts on neutrophils to induce chemotaxis.

Generally, inflammatory disorders include, but are not limited to, respiratory disorders (including asthma, COPD, chronic bronchitis and cystic fibrosis); cardiovascular related disorders (including atherosclerosis, post-angioplasty, restenosis, coronary artery diseases and angina); inflammatory diseases of the joints (including rheumatoid and osteoarthritis); skin disorders (including dermatitis, eczematous dermatitis and psoriasis); post transplantation late and chronic solid organ rejection; multiple sclerosis; autoimmune conditions (including systemic lupus erythematosus, dermatomyositis, polymyositis, Sjogren's syndrome, polymyalgia rheumatica, temporal arteritis, Behcet's disease, Guillain Barré, Wegener's granulomatosus, polyarteritis nodosa); inflammatory neuropathies (including inflammatory polyneuropathies); vasculitis (including Churg-Strauss syndrome, Takayasu's arteritis); inflammatory disorders of adipose tissue; and proliferative disorders (including Kaposi's sarcoma and other proliferative disorders of smooth muscle cells).

### Respiratory Disorders

In one embodiment, compounds, compositions and methods of treatment of respiratory disorders comprising administering a compound are provided wherein the compound is as described herein. Respiratory disorders that may be prevented or treated include a disease or disorder of the respiratory system that can affect any part of the respiratory tract. These conditions range from life threatening to mild. Certain diseases cause respiratory symptoms although the diseases are initially caused by an infection, such as a cold virus, bronchitis, pneumonia and tuberculosis. Other disorders are caused by irritation of the lung tissue, such as, for example, by an allergen. These disorders include hay fever and other respiratory allergies and asthma. In certain embodiments, the host is at risk of or suffering from a disorder of the lower airway. These include bronchitis, simple and mucopurulent chronic bronchitis, unspecified chronic bronchitis (including chronic bronchitis NOS, chronic tracheitis and chronic tracheobronchitis), emphysema, other chronic obstructive pulmonary disease, asthma, status asthmaticus and bronchiectasis.

In asthma, the bronchi and bronchioles are typically temporarily constricted and inflamed. Other disorders typically involving lung irritants include emphysema, which can result from multiple factors including: smog, cigarette smoke, infection, and a genetic predisposition to the condition, laryngitis, lung cancer, respiratory distress syndrome (RDS), which refers to a group of symptoms that indicate severe malfunctioning of the lungs affecting adults and infants and specifically Adult respiratory distress syndrome (ARDS). Chronic respiratory insufficiency (or chronic obstructive pulmonary disease; COPD) is a prolonged or persistent condition characterized by breathing or respiratory dysfunction resulting in reduced rates of oxygenation or the ability to eliminate carbon dioxide.

The term “asthma” as used herein includes any asthmatic condition marked by recurrent attacks of paroxysmal dyspnea (i.e., “reversible obstructive airway passage disease”) with wheezing due to spasmodic contraction of the bronchi (so called “bronchospasm”). Asthmatic conditions which may be treated or even prevented in accordance with this invention include allergic asthma and bronchial allergy characterized by manifestations in sensitized persons provoked by a variety of factors including exercise, especially vigorous exercise (“exercise-induced bronchospasm”), irritant particles (pollen, dust, cotton, cat dander) as well as mild to moderate asthma, chronic asthma, severe chronic asthma, severe and unstable asthma, nocturnal asthma, and psychologic stresses.

Other respiratory disorders include allergic and non-allergic rhinitis as well as non-malignant proliferative and/or inflammatory disease of the airway passages and lungs. Allergic rhinitis means generally any allergic reaction of the nasal mucosa and includes hay fever (seasonal allergic rhinitis) and perennial rhinitis (non-seasonal allergic rhinitis) which are characterized by seasonal or perennial sneezing, rhinorrhea, nasal congestion, pruritis and eye itching, redness and tearing. Non-allergic rhinitis means eosinophilic nonallergic rhinitis which is found in patients with negative skin tests and those who have numerous eosinophils in their nasal secretions.

Non-malignant prolifertive and/or inflammatory diseases of the airway passages or lungs means one or more of (1) alveolitis, such as extrinsic allergic alveolitis, and drug toxicity such as caused by, e.g. cytotoxic and/or alkylating agents; (2) vasculitis such as Wegener's granulomatosis, allergic granulomatosis, pulmonary hemangiomatosis and idiopathic pulmonary fibrosis, chronic eosinophilic pneumonia, eosinophilic granuloma and sarcoidoses.

In one embodiment, the use of the compounds of the invention reduces symptoms of these disorders, including cough, shortness of breath, chest pain, wheezing, cyanosis, finger clubbing, stridor (a crowing sound when breathing), hemoptysis (coughing up of blood), and respiratory failure. The use of these compounds may reduce respiratory acidosis, due to a failure by the lungs to remove carbon dioxide.

In another embodiment, the use of the compounds improve lung function.

Cardiovascular Related Disorders

In one embodiment, the compounds of the invention are administered to a patient suffering from a cardiovascular disorder related to inflammation. These include, but are not limited to, atherosclerosis, post-angioplasty restenosis, coronary artery diseases and angina.

Generally, cardiovascular disorders are a class of diseases that involve the heart and/or blood vessels (arteries and veins). While the term technically refers to any disease that affects the cardiovascular system, it is usually used to refer to those related to atherosclerosis (arterial disease).

Cardiovascular inflammatory disorders include atherosclerosis, post-angioplasty, restenosis, coronary artery diseases, angina, and other cardiovascular diseases. In certain embodiments the disorder is a non-cardiovascular inflammatory disorder such as rheumatoid and osteoarthritis, dermatitis, psoriasis, cystic fibrosis, post transplantation late and chronic solid organ rejection, eczematous dermatitis, Kaposi's sarcoma, or multiple sclerosis. In yet another embodiment, the compounds disclosed herein can be selected to treat anti-inflammatory conditions that are mediated by mononuclear leucocytes. In an alternative embodiment, the compounds can be administered to treat small vessel disease that is not treatable by surgery or angioplasty, or other vessel disease in which surgery is not an option. The compounds can also be used to stabilize patients prior to revascularization therapy.

Generally, unstable atherosclerotic plaque is a result of multiple factors but is commonly characterized by an infiltrate of inflammatory cells. Medical research strongly supports a role for inflammation in the pathogenesis, progression, and disruption of atherosclerotic plaque. Clinical studies have demonstrated systemic markers of inflammation to be strong predictors of clinical events, and specific treatments of atherosclerosis and its risk factors have been associated with reductions in inflammatory markers. The majority of cardiovascular events occur at sites of “nonsignificant” stenosis, as inflammation can lead to instability and rupture of these smaller atherosclerotic plaques, which are more numerous than the “significant,” flow-limiting plaques. In fact, direct visualization of inflammatory cells within plaques is a predictor of unstable coronary disease. The source of inflammation is uncertain; various infectious agents have been proposed as a stimulator of this inflammatory process. Smooth muscle cell proliferation is also implicated both in chronic cardiovascular pathologies such as atherosclerosis, and more directly in, for example, post-angioplasty restenosis.

Diseases of arteries, arterioles and capillaries generally include atherosclerosis, peripheral vascular diseases including Raynaud's syndrome, thromboangiitis obliterans (Buerger) and other specified peripheral vascular diseases such as intermittent claudication.

Proliferative Disorders

Chronic inflammation is a risk factor for many proliferative disorders. For example, in a variety of diseases, airway smooth muscle mass increases due to the coordinated increase in size (hypertrophy) and number (hyperplasia) of airway smooth muscle cells. Myocyte migration may also serve to regulate airway smooth muscle mass. For example, chronic cellular inflammation and airway wall remodelling with subepithelial fibrosis and airway smooth muscle (ASM) cell hyperplasia are features of chronic asthma. In addition, vascular smooth muscle, and immune cells are stimulated in cardiovascular disorders.

In particular, inflammation is a risk factor in development of cancers, including colon cancer, and data from experimental and observational studies suggest that inflammation acts early in the carcinogenic pathway of colorectal cancer, possibly promoting the progression of colorectal adenomas to adenocarcinoma (Tangrea et al. Non-steroidal anti-inflammatory drug use is associated with reduction in the recurrence of advanced and non-advanced colorectal adenomas. Cancer Causes Control 2003; 14:403-11; Dranoff G. Cytokines in cancer pathogenesis and cancer therapy. Nat Rev Cancer 2004; 4:11-22; O'Byrne et al. Chronic immune activation and inflammation as the cause of malignancy. Br J Cancer 2001; 85:473-783; Balkwill et al. Inflammation and cancer: back to Virchow Lancet 2001; 357:539-45; Coussens et al. Inflammation and cancer. Nature 2002; 420:860-7). The inflammatory response to cellular stresses, injury and infection, results from increased mucosal production of proinflammatory cytokines. Proinflammatory cytokines, such as tumor necrosis factor α and the interleukins (IL-1β, IL-6, and IL-8), play a key role in angiogenesis, inhibition of apoptosis, and cell proliferation. These cytokines induce expression of cyclooxygenase 2 (COX-2), one of the key enzymes in the production of prostaglandins. COX-2 mRNA and protein are present in both colorectal adenomas and adenocarcinomas, and thus support a role of inflammation early in the carcinogenic pathway of colorectal cancer.

Other Inflammatory Disorders

In another embodiment, the compounds of the invention may be administered for the treatment or prophylaxis of an inflammatory disorder or the joints or connective tissue. These disorders include rheumatoid arthritis, lupus erythematosus, Sjögren's syndrome, scleroderma (systemic sclerosis), dermatomyositis, polychondritis, polymyositis, polymyalgia rheumatica, osteoarthritis, septic arthritis, fibromyalgia, gout, pseudogout, spondyloarthropathies, such as ankylosing spondylitis, reactive arthritis (Reiter's syndrome), psoriatic arthropathy, enteropathic spondylitis and reactive arthropathy, vasculitis, such as polyarteritis nodosa, Henoch-Schönlein purpura, serum sickness, Wegener's granulomatosis, giant cell arteritis, temporal arteritis, Takayasu's arteritis, Behçcet's syndrome, Kawasaki's disease (mucocutaneous lymph node syndrome) and Buerger's disease (thromboangiitis obliterans). In addition, autoimmune conditions such as acute disseminated encephalomyelitis, Addison's disease, ankylosing spondylitisis, antiphospholipid antibody syndrome, autoimmune hepatitis, Coeliac disease, Crohn's disease, diabetes mellitus, Graves' disease, Guillain-Barré syndrome, Hashimoto's disease, idiopathic thrombocytopenic purpura, Kawasaki's Disease, lupus erythematosus, multiple sclerosis, Mmyasthenia gravis, opsoclonus myoclonus syndrome, optic neuritis, Ord's thyroiditis, pemphigus, pernicious anaemia, primary biliary cirrhosis, Reiter's syndrome, Sjögren's syndrome, Takayasu's arteritis, temporal arteritis, warm autoimmune hemolytic anemia and Wegener's granulomatosis.

In other embodiments, certain inflammtory skin disorders are treated or prevented, such as dermatitis, eczematous dermatitis and psoriasis. In general inflammatory skin disease is a broad category that includes many conditions, ranging in severity from mild itching to serious medical health complications. Other conditions that are inflammatory skin disorders include eczema generally, acne and rosacea.

Other disorders may also be treated or prophylactically prevented or reduced by administration of compounds of the invention. In certain embodiments, the disorder to be treated is selected from post transplantation late and chronic solid organ rejection; multiple sclerosis; autoimmune conditions (including systemic lupus erythematosus, dermatomyositis, polymyositis, inflammatory neuropathies (Guillain Barré, inflammatory polyneuropathies), vasculitis (Wegener's granulomatosus, polyarteritis nodosa), and rare disorders such as polymyalgia rheumatica, temporal arteritis, Sjogren's syndrome, Bechet's disease, Churg-Strauss syndrome, and Takayasu's arteritis).

Diabetes

Methods and pharmaceutical compositions are provided for the treatment or prophylaxis or delay of onset of diabetes, pre-diabetes and related disorders. Related disorders of diabetes includes, but is not limited to, hyperglycemia, abnormal glucose homeostasis, insulin resistance, Syndrome X, metabolic disorders, diabetic dyslipidemia.

In one embodiment, the disease to be treated or prevented is type 2 diabetes. The chronic overabundance of glucose associated with diabetes damages the body's blood vessels and can lead to many related disorders. Generally, high glucose levels in the blood plasma (hyperglycemia) can lead higher than normal amounts of particular hemoglobin, HbA1c. Persistent or uncontrolled hyperglycemia that occurs with diabetes is associated with increased and premature morbidity and mortality. Often abnormal glucose homeostasis is associated with obesity, hypertension, and alterations of the lipid, lipoprotein and apolipoprotein metabolism, as well as other metabolic and hemodynamic disease. Patients with type 2 diabetes mellitus have a significantly increased risk of macrovascular and microvascular complications, including atherosclerosis, coronary heart disease, stroke, peripheral vascular disease, hypertension, nephropathy, neuropathy, microangiopathy, kidney disorders or failure, kidney and nerve damage, cardiac disease, diabetic retinopathy and other ocular disorders, including blindness. In extreme cases, diabetes can result in the amputation of limbs and death.

Other conditions related to diabetes reported by the CDC include: nervous system diseases, which often includes impaired sensation or pain in the feet or hands, slowed digestion of food in the stomach, carpal tunnel syndrome, and other nerve problems, periodontal disease, which is a type of gum disease that can lead to tooth loss, complications of pregnancy, including congenital malformations and death of the fetus, and other complications such as diabetic ketoacidosis and hyperosmolar nonketotic coma.

Many patients who have insulin resistance or type 2 diabetes often have several symptoms that together are referred to as syndrome X, or the metabolic syndrome. A patient having this syndrome is characterized as having three or more symptoms selected from the following group of five symptoms: (1) abdominal obesity; (2) hypertriglyceridemia; (3) low high-density lipoprotein cholesterol (HDL); (4) high blood pressure; and (5) elevated fasting glucose, which may be in the range characteristic of Type 2 diabetes if the patient is also diabetic. Each of these symptoms is defined in the recently released Third Report of the National Cholesterol Education Program Expert Panel on Detection, Evaluation and Treatment of High Blood Cholesterol in Adults (Adult Treatment Panel III, or ATP III), National Institutes of Health, 2001, NIH Publication No. 01-3670.

In one embodiment, the compound is provided to a host to promote depletion of bile salts. Bile salts are steroids with detergent properties which are used to emulsify lipids in foodstuff passing through the intestine to enable fat digestion and absorption through the intestinal wall. They are secreted from the liver stored in the gall bladder and passed through the bile duct into the intestine when food is passing through. The most abundant of the bile salts in humans are cholate and deoxycholate, and they are normally conjugated with either glycine or taurine to give glycocholate or taurocholate respectively. Depletion of bile salts, including cholate and deoxycholate, force the liver to reabsorb cholesterol to make new bile.

In one embodiment, patients at risk for developing diabetes are prophylactically treated to prevent onset. Patients with diabetes or at risk for developing diabetes can be identified through several risk factors. One of the key risk factors is age and obesity. Generally patients who are 45 years or older and overweight (with a body mass index of 25 or greater) is at risk of developing diabetes.

Additional risk factors for type 2 diabetes include a family history, ethnicity (Alaska Native, American Indian, African American, Hispanic/Latino, Asian American, or Pacific Islander is at higher risk), having had gestational diabetes or giving birth to a baby weighing more than 9 pounds, previous history of high blood pressure or blood pressure of 140/90 mm Hg or higher, cholesterol levels not normal (including HDL below 35 mg/dL, or triglyceride level above 250 mg/dL), being fairly inactive (less than three times per week exercise), diagnosis of polycystic ovary syndrome, any test showing impaired glucose tolerance (IGT) or impaired fasting glucose (IFG), clinical conditions associated with insulin resistance, such as acanthosis nigricans, or a history of cardiovascular disease. Tests to be conducted can include a fasting blood glucose test or an oral glucose tolerance test.

Glucose levels of approximately 100-126 mg/dl in a fasting plasma glucose test (FPG) or approximately 140-200 mg/dl in the oral glucose tolerance test (OGTT) indicate pre-diabetes. Levels of greater than or equal to 126 mg/dl in the FPG or greater than or equal to 200 mg/dl in the OGTT indicate diabetes. Symptoms of diabetes include increased thirst, increased hunger, fatigue, increased urination, especially at night, weight loss, blurred vision, sores that do not heal.

### Pharmaceutical Compositions

Mammals, and specifically humans, suffering from an inflammatory disorder, including any of the above-described conditions, and in particular suffering from respiratory disorders, can be treated by either targeted or systemic administration, via oral, inhalation, topical, trans- or sub-mucosal, subcutaneous, parenteral, intramuscular, intravenous or transdermal administration of a composition comprising an effective amount of the compounds described herein or a pharmaceutically acceptable salt, ester or prodrug thereof, optionally in a pharmaceutically acceptable carrier.

The compounds or composition is typically administered by oral administration. Alternatively, compounds can be administered by inhalation. In another embodiment, the compound is administered transdermally (for example via a slow release patch), or topically. In yet another embodiment, the compound is administered subcutaneously, intravenously, intraperitoneally, intramuscularly, parenterally, or submucosally. In any of these embodiments, the compound is administered in an effective dosage range to treat the target condition.

In one embodiment, compounds of the present invention are administered orally. Oral compositions will generally include an inert diluent or an edible carrier. They may be enclosed in gelatin capsules or compressed into tablets. For the purpose of oral therapeutic administration, the active compound can be incorporated with excipients and used in the form of tablets, troches, or capsules. Pharmaceutically compatible binding agents, and/or adjuvant materials can be included as part of the composition.

When the compound is administered orally in the form of a dosage unit such as a tablets, pills, capsules, troches and the like, these can contain any of the following ingredients, or compounds of a similar nature: a binder (such as microcrystalline cellulose, gum tragacanth or gelatin); an excipient (such as starch or lactose), a disintegrating agent (such as alginic acid, Primogel, or corn starch); a lubricant (such as magnesium stearate or Sterotes); a glidant (such as colloidal silicon dioxide); a sweetening agent (such as sucrose or saccharin); and/or a flavoring agent (such as peppermint, methyl salicylate, or orange flavoring). When the dosage unit form is a capsule, it can contain, in addition to material of the above type, a liquid carrier (such as a fatty oil). In addition, dosage unit forms can contain various other materials which modify the physical form of the dosage unit, for example, coatings of sugar, shellac, or other enteric agents.

The compound or its salts can also be administered orally as a component of an elixir, suspension, syrup, wafer, chewing gum or the like. A syrup may contain, in addition to the active compounds, a sweetening agent (such as sucrose, saccharine, etc.) and preservatives, dyes and colorings and flavors.

The compounds of the invention may be also administered in specific, measured amounts in the form of an aqueous suspension by use of a pump spray bottle. The aqueous suspension compositions of the present invention may be prepared by admixing the compounds with water and other pharmaceutically acceptable excipients. The aqueous suspension compositions according to the present invention may contain, inter alia, water, auxiliaries and/or one or more of the excipients, such as: suspending agents, e.g., microcrystalline cellulose, sodium carboxymethylcellulose, hydroxpropyl-methyl cellulose; humectants, e.g. glycerin and propylene glycol; acids, bases or buffer substances for adjusting the pH, e.g., citric acid, sodium citrate, phosphoric acid, sodium phospate as well as mixtures of citrate and phosphate buffers; surfactants, e.g. Polysorbate 80; and antimicrobial preservatives, e.g., benzalkonium chloride, phenylethyl alcohol and potassium sorbate.

In a separate embodiment, the compounds of the invention are in the form of an inhaled dosage. In this embodiment, the compounds may be in the form of an aerosol suspension, a dry powder or liquid particle form. The compounds may be prepared for delivery as a nasal spray or in an inhaler, such as a metered dose inhaler. Pressurized metered-dose inhalers (“MDI”) generally deliver aerosolized particles suspended in chlorofluorocarbon propellants such as CFC-11, CFC-12, or the non-chlorofluorocarbons or alternate propellants such as the fluorocarbons, HFC-134A or HFC-227 with or without surfactants and suitable bridging agents. Dry-powder inhalers can also be used, either breath activated or delivered by air or gas pressure such as the dry-powder inhaler disclosed in the Schering Corporation International Patent Application No. PCT/US92/05225, published 7 Jan. 1993 as well as the Turbuhaler™ (available from Astra Pharmaceutical Products, Inc.) or the Rotahaler™ (available from Allen & Hanburys) which may be used to deliver the aerosolized particles as a finely milled powder in large aggregates either alone or in combination with some pharmaceutically acceptable carrier e.g. lactose; and nebulizers.

Solutions or suspensions used for parenteral, intradermal, subcutaneous, or topical application can include at least some of the following components: a sterile diluent (such as water for injection, saline solution, fixed oils, polyethylene glycols, glycerine, propylene glycol or other synthetic solvents); antibacterial agents (such as benzyl alcohol or methyl parabens); antioxidants (such as ascorbic acid or sodium bisulfite); chelating agents (such as ethylenediaminetetraacetic acid); buffers (such as acetates, citrates or phosphates); and/or agents for the adjustment of tonicity (such as sodium chloride or dextrose). The pH of the solution or suspension can be adjusted with acids or bases, such as hydrochloric acid or sodium hydroxide.

A parenteral preparation can be enclosed in ampoules, disposable syringes or multiple dose vials made of glass or plastic.

Suitable vehicles or carriers for topical application can be prepared by conventional techniques, such as lotions, suspensions, ointments, creams, gels, tinctures, sprays, powders, pastes, slow-release transdermal patches, suppositories for application to rectal, vaginal, nasal or oral mucosa. In addition to the other materials listed above for systemic administration, thickening agents, emollients, and stabilizers can be used to prepare topical compositions. Examples of thickening agents include petrolatum, beeswax, xanthan gum, or polyethylene, humectants such as sorbitol, emollients such as mineral oil, lanolin and its derivatives, or squalene.

If administered intravenously, carriers can be physiological saline, bacteriostatic water, Cremophor EL™ (BASF, Parsippany, N.J.) or phosphate buffered saline (PBS).

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) are also preferred as pharmaceutically acceptable carriers. These may be prepared according to methods known to those skilled in the art, for example, as described in U.S. Pat. No. 4,522,811 (which is incorporated herein by reference in its entirety). For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the compound is then introduced into the container. The container is then swirled by hand to free lipid material from the sides of the container and to disperse lipid aggregates, thereby forming the liposomal suspension.

Dosing

The compound is administered for a sufficient time period to alleviate the undesired symptoms and the clinical signs associated with the condition being treated. In one embodiment, the compounds are administered less than three times daily. In one embodiment, the compounds are administered in one or two doses daily. In one embodiment, the compounds are administered once daily. In some embodiments, the compounds are administered in a single oral dosage once a day.

The active compound is included in the pharmaceutically acceptable carrier or diluent in an amount sufficient to deliver to a patient a therapeutic amount of compound in vivo in the absence of serious toxic effects. An effective dose can be readily determined by the use of conventional techniques and by observing results obtained under analogous circumstances. In determining the effective dose, a number of factors are considered including, but not limited to: the species of patient; its size, age, and general health; the specific disease involved; the degree of involvement or the severity of the disease; the response of the individual patient; the particular compound administered; the mode of administration; the bioavailability characteristics of the preparation administered; the dose regimen selected; and the use of concomitant medication.

Typical systemic dosages for the herein described conditions are those ranging from 0.01 mg/kg to 1500 mg/kg of body weight per day as a single daily dose or divided daily doses. Preferred dosages for the described conditions range from 0.5-1500 mg per day. A more particularly preferred dosage for the desired conditions ranges from 5-750 mg per day. Typical dosages can also range from 0.01 to 1500, 0.02 to 1000, 0.2 to 500, 0.02 to 200, 0.05 to 100, 0.05 to 50, 0.075 to 50, 0.1 to 50, 0.5 to 50, 1 to 50, 2 to 50, 5 to 50, 10 to 50, 25 to 50, to 75, 25 to 100, 100 to 150, or 150 or more mg/kg/day, as a single daily dose or divided daily doses. In one embodiment, the daily dose is between 10 and 500 mg/day. In another embodiment, the dose is between about 10 and 400 mg/day, or between about 10 and 300 mg/day, or between about 20 and 300 mg/day, or between about 30 and 300 mg/day, or between about 40 and 300 mg/day, or between about 50 and 300 mg/day, or between about 60 and 300 mg/day, or between about 70 and 300 mg/day, or between about 80 and 300 mg/day, or between about 90 and 300 mg/day, or between about 100 and 300 mg/day, or about 200 mg/day. In one embodiment, the compounds are given in doses of between about 1 to about 5, about 5 to about 10, about 10 to about 25 or about 25 to about 50 mg/kg. Typical dosages for topical application are those ranging from 0.001 to 100% by weight of the active compound.

The concentration of active compound in the drug composition will depend on absorption, inactivation, and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the dosage ranges set forth herein are exemplary only and are not intended to limit the scope or practice of the claimed composition. The active ingredient may be administered at once, or may be divided into a number of smaller doses to be administered at varying intervals of time.

### Combination Treatment

The compound can also be mixed with other active materials which do not impair the desired action, or with materials that supplement the desired action. The active compounds can be administered in conjunction, i.e. combination or alternation, with other medications used in the treatment of respiratory disorders. In another embodiment, the compounds can be administered in conjunction (combination or alternation) with other medications used in treatment or prophylaxis of inflammatory conditions. In certain embodiments, the combination can be synergistic.

In one embodiment, the compounds can be administered in combination or alternation with drugs typically useful for treatment or prevention of respiratory conditions such as asthma, such as certain anti-inflammatory drugs and bronchodilators. Corticosteroids (inhaled and oral), mast cell stabilizers, and the leukotriene modifier drugs are typically a useful anti-inflammatory medication for people suffering from asthma. These drugs reduce swelling and mucus production in the airways. Bronchodilators typically relieve the symptoms of asthma by relaxing the muscle bands that tighten around the airways. This action rapidly opens the airways, letting more air come in and out of the lungs. Bronchodilators also help clear mucus from the lungs.

Typically used compounds include inhaled corticosteroids, which prevent rather than relieve symptoms. Inhaled corticosteroids include: Advair (a combination medication that includes a corticosteroid (fluticasone) plus a long acting bronchodilator drug (in this case a β-2 adrenergic receptor agonist, salmeterol)), aerobid (flunisolide), azmacort (triamcinolone), flovent (fluticasone), methylprednisolone, prednisone, pulmicort or serevent diskus (salmeterol powder), theophylline, qvar, and xopenex (levalbuterol), Inhaled corticosteroids come in three forms: the metered dose inhaler (MDI), dry powder inhaler (DPI) and nebulizer solutions. Systemic steroids include: methylprednisolone (Medrol, Methylpred, Solu-Medrol), prednisone (Deltasone) and prednisolone (Prelone, Pediapred, Orapred). Mast Cell Stabilizers include Intal and Tilade, which work by preventing the release of irritating and inflammatory substances from mast cells. Leukotriene modifiers include accolate and singular and accolate (zafirlukast), singulair (montelukast) and zyflo (zileuton).

The compounds can be administered in combination with nonsteroidal antiinflammatories such as ibuprofen, indomethacin, fenoprofen, mefenamic acid, flufenamic acid, sulindac. The compound can also be administered with corticosteriods. Any of the compounds described herein for combination or alternation therapy can be administered as any prodrug that upon administration to the recipient, is capable of providing directly or indirectly, the parent compound. Nonlimiting examples are the pharmaceutically acceptable salts (alternatively referred to as “physiologically acceptable salts”), and a compound which has been alkylated or acylated at an appropriate position. The modifications can affect the biological activity of the compound, in some cases increasing the activity over the parent compound.

In another embodiment, the active compounds can be administered in conjunction with medications used in the treatment or prophylaxis of conditions associated with cardiovascular disease. These compounds include lipid lowering agents, such as statins, probucol and nicotinic acid; platelet aggregation inhibitors such as aspirin; antithrombotic agents such as coumadin; calcium channel blockers such as varapamil, diltiazem, and nifedipine; angiotensin converting enzyme (ACE) inhibitors such as captopril and enalopril, and β-blockers such as propanalol, terbutalol, and labetalol. The compounds can also be administered in combination with nonsteroidal antiinflammatories such as ibuprofen, indomethacin, fenoprofen, mefenamic acid, flufenamic acid, sulindac. The compound can also be administered, for example, with corticosteriods.

In some embodiments, the compounds are administered in combination or alternation with ACE (angiotensin-converting enzyme) inhibitors. Nonlimiting examples are captopril (Capoten), enalapril (Vasotec), lisinopril (Prinivil, Zestril), quinapril (Accupril), ramipril (Altace), benazepril (Lotensin) and fosinopril (Monopril). In another embodiment, the compounds are administered in combination or alternation with beta blockers. Nonlimiting examples are atenolol (Tenormin), carvedilol (Coreg), labetolol (Normodyne), metoprolol (Lopressor, Toprol) and propanolol (Inderal). In another embodiment, the compounds are administered in combination or alternation with blood thinners such as aspirin or warfarin (Coumadin) or calcium channel blockers such as amlodipine (Norvasc), diltiazem (Cardizem, Dilacor), nifedipine (Adalat, Procardia), nicardipine (Cardene) or verapamil (Calan). In another embodiment, the compounds are administered in combination or alternation with a statin. Nonlimiting examples of currently used statins are lovastatin (Mevacor, Altocor), pravastatin (Pravachol), simvastatin (Zocor), fluvastatin (Lescol), atorvastatin (Lipitor).

The compounds can also be administered in combination or alternation with compounds that are generally used for treatment of skin inflammatory conditions, such as Acitretin, Alclometasone dipropionate, Allantoin/Coal tar extract/Hydrocortisone, Alphademm, Alphosyl HC, Asmanex, Benzalkonium chloride/Dimeticone 350/Hydrocortisone/Nystatin, Betacap, Betamethasone dipropionate, Betamethasone dipropionate/Calcipotriol hydrate, Betamethasone dipropionate/Salicylic acid, Betamethasone Valerate, Betamethasone Valerate/Clioquinol, Betamethasone Valerate/Fusidic Acid, Betamethasone valerate/Neomycin sulphate, Betnovate, Betnovate-C, Betnovate-N, Bettamousse, Calcipotriol, Calcipotriol hydrate, Calcitriol, Calmurid HC, Canesten HC, Chlorquinaldol/Hydrocortisone Butyrate, Ciclosporin, Clarelux, Clioquinol/Hydrocortisone, Clobetasol propionate, Clobetasol propionate/Neomycin sulphate/Nystatin, Clobetasone butyrate, Clobetasone butyrate/Nystatin/Oxytetracycline calcium, Clotrimazole/Hydrocortisone, Crotamiton/Hydrocortisone, Cutivate, Daktacort, Dandrazol, Dermovate, Dermovate-NN, Dioderm, Diprosalic, Diprosone, Dithranol, Dithrocream, Dovobet, Dovonex, Dovonex cream Econacort, Econazole nitrate/Hydrocortisone, Efalizumab, Efcortelan, Elidel, Enbrel, Etanercept, Eumovate, Eurax Hydrocortisone, Fluticasone propionate, Fucibet, Fucidin H, Fucidin H ointment, Fusidic acid/Hydrocortisone acetate, Gramicidin/Neomycin sulphate/Nystatin/Triamcinolone acetonide, Hydrocortisone, Hydrocortisone acetate/Sodium fusidate, Hydrocortisone butyrate, Hydrocortisone/Lactic Acid/Urea, Hydrocortisone/Miconazole nitrate, Hydrocortisone/Urea, Infliximab, Kenalog, Ketoconazole, Locoid, Locoid C, Maxtrex, Methotrexate, Methotrexate sodium, Modrasone, Mometasone, Nasofan, Neoral, Neotigason, Nizoral, Pimecrolimus, Protopic, Raptiva, Remicade, Silkis, Tacrolimus monohydrate, Tazarotene, Timodine, Tri-Adcortyl, Triamcinolone acetonide, Trimovate, Vioform-Hydrocortisone and Zorac.

Any of the compounds described herein for combination or alternation therapy can be administered as any prodrug that upon administration to the recipient, is capable of providing directly or indirectly, the parent compound. Nonlimiting examples are the pharmaceutically acceptable salts (alternatively referred to as “physiologically acceptable salts”), and a compound which has been alkylated or acylated at an appropriate position. The modifications can affect the biological activity of the compound, in some cases increasing the activity over the parent compound. This can easily be assessed by preparing the derivative and testing its ability to inhibit the expression of VCAM-1 according to known methods.

### General Synthesis

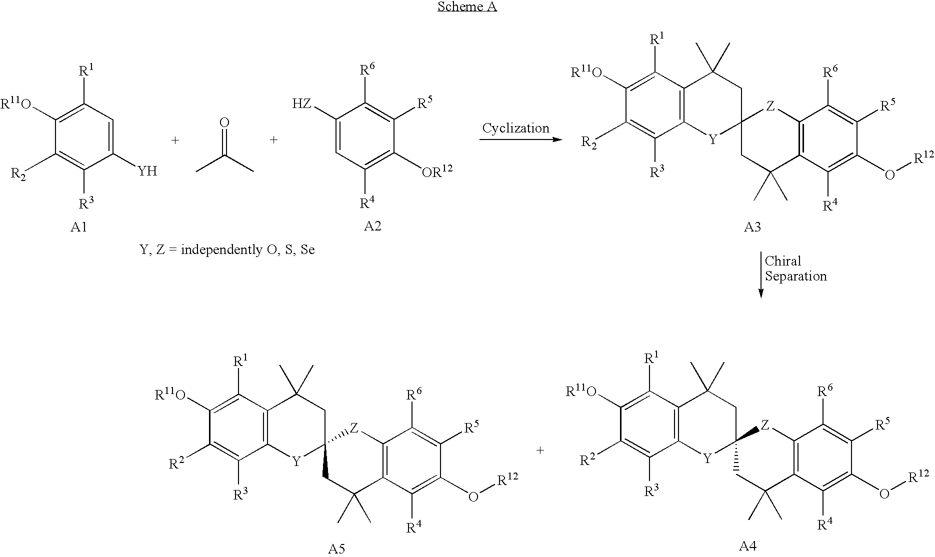

Step 1: A compound of formula A1 and a compound of formula A2 are reacted in a an organic solvent, for example acetone, in the presence of an acid catalyst such as sulfuric acid, hydrochloric acid, glacial acetic acid, and the like, to yield compound of formula A3.

Step 2: Compound of formula A3 can be separated into pure (>95% e.e.) enantiomers, A4 and A5 by HPLC, SFC, and the like, using a chiral stationary phase such as Whelk-O, ChiralPak AD, ChiralPak OD, and the like, with an alcoholic solvent such as methanol, ethanol, isopropanol, and the like, or a mixture of alcoholic solvents, as the eluant.

Alternatively, compound of formula A3 can be resolved into enantiomers, A4 and A5 by forming a diastereomeric salt with a chiral amine such as brucine, quinine, cinchonine, ephedrine, alpha-methylbenzylamine, and the like, in a crystallization solvent such as water, methanol, ethanol, acetonitrile, ethylene glycol, and the like, or a mixture of crystallization cosolvents, comprising of water, methanol, ethanol, acetonitrile, ethylene glycol, and the like.

Furthermore, hydrolysis of ester substituents on R¹² in A3, A4 and A5 can be carried out under basic conditions, for example, by treatment with aqueous solutions of sodium hydroxide or potassium hydroxide in an alcoholic solvent such as 2-methoxy-ethanol, ethanol, or methanol, or an ethereal solvent such as THF, 1,4-dioxane, and the like, at a temperature range of about ambient temperature to 150° C.

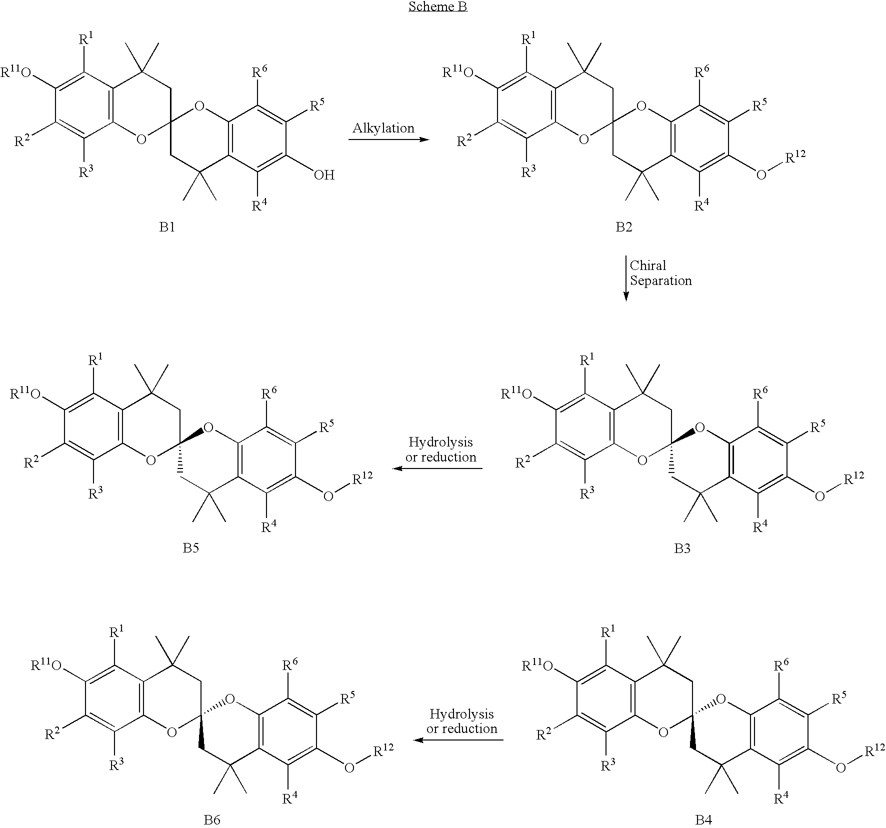

Step 1: Compound of formula B1 is alkylated with a suitably substituted alkylating reagent in the presence of an organic base such as diisopropylethylamine, pyridine, potassium tert-butoxide, benzylmagnesium halide, triethylamine, and the like, or an inorganic base such as potassium carbonate, cesium carbonate, sodium carbonate and the like, in an organic solvent such as THF, 1,4-dioxane, dichloromethane, toluene, DMF, and the like, at a temperature range of about 20-80° C. to yield the compound of formula B2.

Step 2: Compound of formula B2 is separated into pure (>95% e.e.) enantiomers, B3 and B4 by HPLC, SFC, and the like, using a chiral stationary phase such as Whelk-O, ChiralPak AD, ChiralPak OD, and the like, with an alcoholic solvent such as methanol, ethanol, and the like, as the eluant.

Alternatively, compound of formula B2 can be resolved by forming a diastereomeric salt with a chiral amine such as brucine, quinine, cinchonine, ephedrine, alpha-methylbenzylamine, and the like, in a crystallization solvent such as water, methanol, ethanol, acetonitrile, ethylene glycol, and the like.

Step 3: Hydrolysis of ester substituents on R¹² in B3 or B4 can be carried out under basic conditions, for example, by treatment with aqueous solutions of sodium hydroxide, potassium hydroxide, and the like, in an alcoholic solvent such as 2-methoxyethanol, ethanol, methanol, and the like, at a temperature range from about ambient temperature to 150° C.

Alternatively, reduction of ester substituents on R¹² in B3 or B4 can be carried out under reductive conditions, for example, by treatment with lithium aluminum hydride in an ethereal solvent such as THF or diethylether at a temperature range of about 0° C. to 70° C., or sodium borohydride in an alcoholic solvent such as ethanol, methanol, and the like, at a temperature range of about ambient temperature to 80° C.

Alternatively, addition of alkyl groups can be carried out, for example, by treatment with an alkyl magnesium halide, where the halide is chloride, bromide, iodide or by treatment with an alkyl lithium reagent in an ethereal solvent such as THF, diethylether, and the like, at a temperature range of about 0° C. to 70° C.

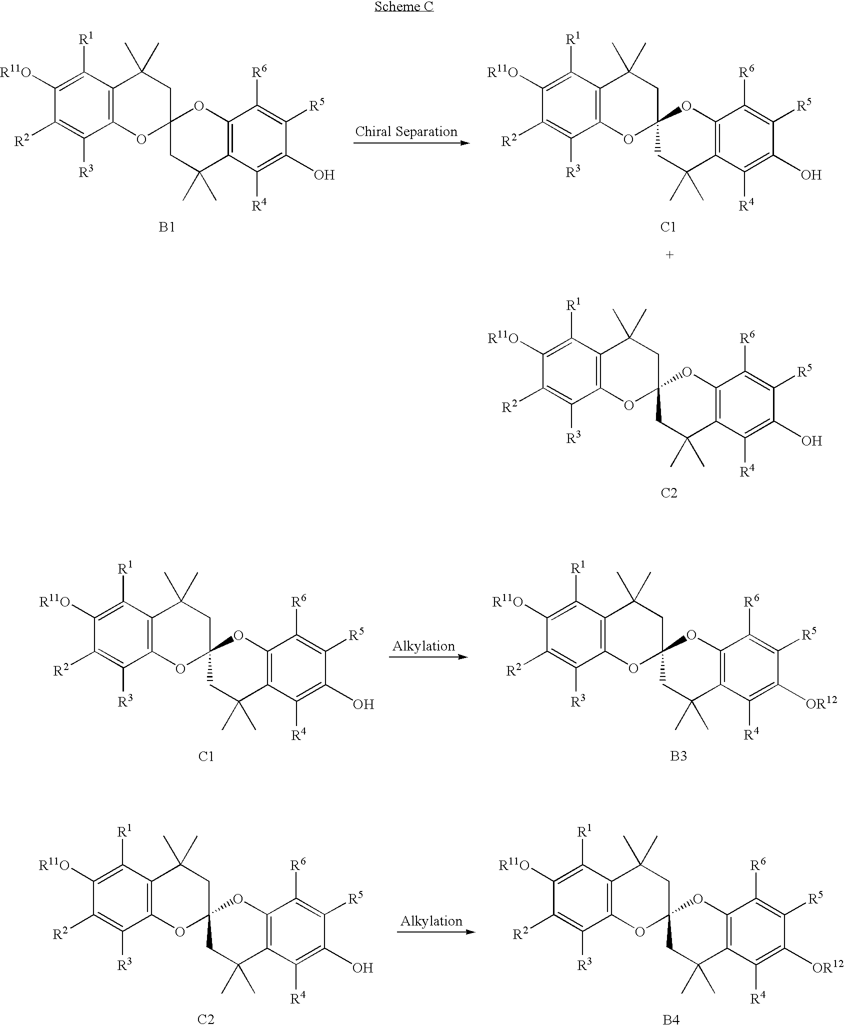

Step 1: Compound of formula B1 is separated into pure (>95% e.e.) enantiomers, C1 and C2 by HPLC, SFC, and the like, using a chiral stationary phase such as Whelk-O, ChiralPak AD, ChiralPak OD, and the like, with an alcoholic solvent such as methanol, ethanol, and the like, as the eluant.

Step 2: Compounds of formula C1 and C2 can be independently alkylated with a suitably substituted alkylating reagent in the presence of an organic base such as diisopropylethylamine, pyridine, potassium tert-butoxide, benzylmagnesium halide, triethylamine, and the like, or an inorganic base such as potassium carbonate, cesium carbonate, sodium carbonate and the like, in an organic solvent such as THF, 1,4-dioxane, dichloromethane, toluene, DMF, and the like, at a temperature range of about 20-80° C. to yield the compound of formula B3 and B4, respectively.

Furthermore, hydrolysis of ester substituents on R¹² in B3 or B4 can be carried out under basic conditions, for example, by treatment with aqueous solutions of sodium hydroxide, potassium hydroxide, and the like, in an alcoholic solvent such as 2-methoxyethanol, ethanol, methanol, and the like, at a temperature range from about ambient temperature to 150° C.

Alternatively, reduction of ester substituents on R¹² in B3 or B4 can be carried out under reductive conditions, for example, by treatment with lithium aluminum hydride in an ethereal solvent such as THF or diethylether at a temperature range of about 0° C. to 70° C., or sodium borohydride in an alcoholic solvent such as ethanol, methanol, and the like, at a temperature range of about ambient temperature to 80° C.

Alternatively, addition of alkyl groups can be carried out, for example, by treatment with an alkyl magnesium halide, where the halide is chloride, bromide, iodide or by treatment with an alkyl lithium reagent in an ethereal solvent such as THF, diethylether, and the like, at a temperature range of about 0° C. to 70° C.

### EXAMPLES

### Example 1

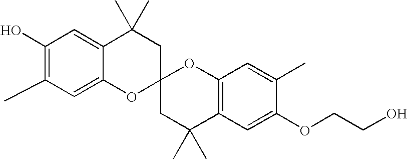

### (±)-6′-Hydroxy-6-(2-hydroxyethoxy)-4,4,4′,4′,7,7′-hexamethyl-2,2′-spirobichroman

To a mixture of (±)-6,6′-dihydroxy-4,4,4′,4′,7,7′-hexamethyl-2,2′-spirobichroman (14.8 g, 40 mmol) in 200 mL of DMF was added K₂CO₃ (5.6 g, 40 mmol). The mixture was heated to 80° C., and then bromoacetic acid ethyl ester (13.6 g, 80 mmol) was added. The resulting mixture was stirred at 80° C. overnight, cooled to room temperature, and poured into water. The mixture was then extracted with dichloromethane and the organic layers were combined, washed with brine, dried over MgSO₄, and concentrated to dryness. The brown oil obtained was dissolved in 500 mL of THF and cooled to 0° C. To this solution was added a solution of lithium aluminum hydride (50 mL, 1.0 M in THF). The resultant slurry was stirred at room temperature for 72 h. The reaction mixture was carefully quenched with saturated Rochelle's salt solution and stirred for 1 h. It was filtered and the filtrate was concentrated to a crude oil, which was dissolved in dichloromethane and washed with water. The layers were separated and the organic layer was concentrated and subjected to silica gel chromatography (hexanes/EtOAc, 2:1) to afford 4.29 g (26%) of the title compound as a white solid, m.p. 186-188° C. ¹H NMR (CDCl₃): δ 6.76 (s, 1H), 6.73 (s, 1H), 6.48 (s, 1H), 6.44 (s, 1H), 4.49 (s, 1H), 4.06 (t, 2H, J=4.2 Hz), 3.97-3.95 (m, 2H), 2.11 (s, 6H), 2.07-1.90 (m, 4H), 1.58 (s, 3H), 1.57 (s, 3H), 1.33 (s, 3H), 1.31 (s, 3H). Anal. Calcd for C₂₅H₃₂O₅.⅓H₂O: C, 71.74; H, 7.87.

Found: C, 71.83; H, 7.97.

### Example 2

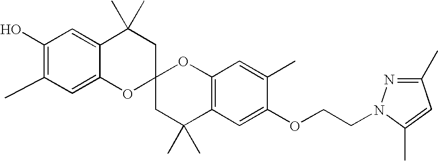

### (±)-6′-Hydroxy-6-[2-(3,5-dimethyl-1H-pyrazol-1-yl)ethoxy]-4,4,4′,4′,7,7′-hexamethyl-2,2′-spirobichroman

Ex. 2a. To (±)-6′-hydroxy-6-(2-hydroxyethoxy)-4,4,4′,4′,7,7′-hexamethyl-2,2′-spirobi-chroman (Ex. 1, 3.44 g, 8.3 mmol) in 100 mL of dichloromethane was added N,N-diiso-propylethylamine (3.26 g, 24.9 mmol), followed by the addition of methanesulfonyl chloride (1.95 ml, 24.9 mmol) at room temperature. The mixture was stirred at room temperature for 18 h. The reaction mixture was washed with water and the organic phase was separated, dried, and concentrated. Crystallization from ethyl acetate and hexanes afforded 4.74 g (100%) of (±)-6′-methanesulfonyloxy-6-(2-methanesulfonyloxyethoxy)-4,4,4′,4′,7,7′ hexamethyl-2,2′-spirobichroman as an off-white solid. ¹H NMR (CDCl₃): δ 7.20 (s, 1H), 6.75 (s, 1H), 6.54 (s, 1H), 6.50 (s, 1H), 4.57 (t, J=4.4 Hz, 2H), 4.22 (t, J=4.4 Hz, 2H), 3.15 (s, 3H), 3.08 (s, 3H), 2.20 (s, 3H), 2.12 (s, 3H), 2.09-1.91 (m, 4H), 1.59 (s, 3H), 1.58 (s, 3H), 1.34 (s, 6H).

Ex. 2b. To 3,5-dimethyl-1H-pyrazole (0.19 g, 2 mmol) in 20 mL of DMF was added sodium hydride (0.12 g, 3 mmol, 60% dispersion in mineral oil) portion-wise. The mixture was stirred at room temperature for 10 min. To this slurry was added (±)-6′-methanesulfonyloxy-6-(2-methanesulfonyloxyethoxy)-4,4,4′,4′,7,7′-hexamethyl-2,2′ spirobichroman (Ex. 2a, 0.57 g, 1 mmol) and the resulting solution was stirred at room temperature for 1 h. The reaction mixture was poured into water and acidified to pH 4 with 0.5 N HCl. The precipitate was filtered, and washed with water. Silica gel chromatography (EtOAc/hexanes, 1:1) gave 0.45 g (79%) of (±)-6′-methanesulfonyloxy-6-[2-(3,5-dimethyl-1H-pyrazol-1-yl)ethoxy]-4,4,4′,4′,7,7′-hexamethyl-2,2′-spirobichrom-an as a white solid. ¹H NMR (CDCl₃): δ 7.19 (s, 1H), 6.62 (s, 1H), 6.53 (s, 1H), 6.44 (s, 1H), 5.78 (s, 1H), 4.35 (t, J=5.1 Hz, 2H), 4.26 (t, J=5.1 Hz, 2H), 3.14 (s, 3H), 2.31 (s, 3H), 2.22 (s, 3H), 2.19 (s, 3H), 2.05 (s, 3H) 2.10-1.89 (m, 4H), 1.58 (s, 3H), 1.54 (s, 3H), 1.33 (s, 3H), 1.30 (s, 3H).

To (±)-6′-methanesulfonyloxy-6-[2-(3,5-dimethyl-1H-pyrazol-1-yl)ethoxy]-4,4,4′,4′,7,7′-hexamethyl-2,2′-spirobichroman (Ex. 2b, 0.45 g, 0.79 mmol) in 10 mL of THF and 10 mL of MeOH was added 5 N NaOH (3 mL) and the resulting mixture was heated at 60° C. overnight. The reaction mixture was diluted with ethyl acetate and washed with water. The layers were separated and the organic layer was dried over MgSO₄ and concentrated. Crystallization from ethyl acetate and hexanes gave 0.30 g (77%) of the title compound as white solid, mp 223-225° C. ¹H NMR (CDCl₃): δ 6.74 (s, 1H), 6.64 (s, 1H), 6.45 (s, 2H), 5.81 (s, 1H), 4.99 (s, 1H), 4.37 (t, J=5.2 Hz, 2H), 4.28 (t, J=5.2 Hz, 2H), 2.34 (s, 3H), 2.25 (s, 3H), 2.20-1.90 (m, 4H), 2.12 (s, 3H), 2.01 (s, 3H), 1.57 (s, 6H), 1.31 (s, 6H). Anal. Calcd for C₃₀H₃₈N₂O₄.⅔H₂O: C, 71.69; H, 7.89; N, 5.57. Found: C, 71.66; H, 7.74; N, 5.54.

### Example 3

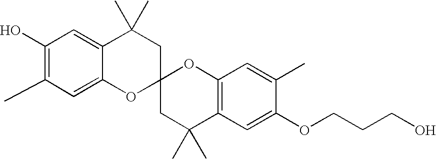

### (±)-6′-Hydroxy-6-(3-hydroxypropoxy)-4,4,4′,4′,7,7′-hexamethyl-2,2′-spirobichroman

To (±)-6,6′-dihydroxy-4,4,4′,4′,7,7′-hexamethyl-2,2′-spirobichroman (7.4 g, 20 mmol) in 100 mL of DMF was added potassium carbonate (5.6 g, 40 mmol), followed by 3-bromo-propanol (5.6 g, 40 mmol). The mixture was stirred at 60° C. for 75 h and then poured into water, acidified to pH 6 with 3 N HCl, and extracted with dichloromethane. The combined organic extracts were washed with brine and water. After concentration, the crude oil was purified via silica gel chromatography using 33% ethyl acetate in hexanes as the eluant to afford the title compound as an off-white solid (2.2 g, 26%), m.p. 175-177° C. ¹H NMR (CDCl₃): δ 6.75 (s, 1H), 6.73 (s, 1H), 6.47 (s, 1H), 6.44 (s, 1H), 4.39 (s, 1H), 4.10 (t, 2H, J=6.0 Hz), 3.90 (br q, 2H, J=5.1 Hz), 2.10 (s, 3H), 2.08 (s, 3H), 2.08-1.90 (m, 6H), 1.58 (s, 3H), 1.51 (s, 3H), 1.33 (s, 3H), 1.31 (s, 3H). Anal. Calcd for C₂₆H₃₄O₅.⅓H₂O: C, 72.19; H, 8.08. Found: C, 72.27; H, 8.05.

### Example 4

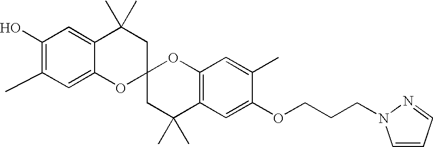

### (±)-6′-Hydroxy-6-[3-(1H-pyrazol-1-yl)propoxy]-4,4,4′,4′,7,7′-hexamethyl-2,2′-spirobichroman

Ex. 4a. To (±)-6′-hydroxy-6-(3-hydroxypropoxy)-4,4,4′,4′,7,7′-hexamethyl-2,2′-spiro-bichroman (Ex. 3, 2.0 g, 4.7 mmol) in 25 mL of dichloromethane was added N,N-diisopropylethylamine (2.42 g, 18.8 mmol), followed by methanesulfonyl chloride (2.14 g, 18.8 mmol). The mixture was stirred at room temperature for 3 h and then diluted with dichloromethane and washed with 0.5 N HCl and brine. The organic phase was separated, dried over MgSO₄, and concentrated. The residue was purified by column chromatography (hexanes/EtOAC, 2:1) and recrystallized from dichloromethane and hexanes to afford 2.0 g (71%) of (±)-6′-methanesulfonyloxy-6-(3-methanesulfonyl-oxypropoxy)-4,4,4′,4′,7,7′ hexamethyl-2,2′-spirobichroman as an off-white solid. ¹H NMR (CDCl₃): δ 7.20 (s, 1H), 6.73 (s, 1H), 6.54 (s, 1H), 6.49 (s, 1H), 4.48 (t, J=5.9 Hz, 2H), 4.06 (t, J=5.9 Hz, 2H), 3.15 (s, 3H), 3.00 (s, 3H), 2.26-2.20 (m, 2H), 2.20 (s, 3H), 2.10 (s, 3H), 2.10-1.90 (m, 4H), 1.59 (s, 3H), 1.58 (s, 3H), 1.34 (s, 6H).

To (±)-6′-methanesulfonyloxy-6-(3-methanesulfonyloxypropoxy)-4,4,4′,4′,7,7′-hexa-methyl-2,2′-spirobichroman (Ex. 4a, 0.8 g, 1.37 mmol) and pyrazole (0.186 g, 2.74 mmol) in 25 mL of DMF at room temperature was added sodium hydride (0.16 g, 4.11 mmol, 60% dispersion in mineral oil) portion-wise. The reaction mixture was stirred at room temperature for 2 h and then poured into water. It was extracted with ethyl acetate and the combined organic extracts were washed with brine and water, and concentrated. The residue obtained was dissolved in 20 mL of THF/MeOH (1:1) and 5N NaOH (3 ml) was added. The resultant mixture was heated to reflux overnight. The reaction mixture was acidified with 3N HCl and extracted with dichloromethane. The organic extracts were concentrated and recrystallized from ethyl acetate and hexanes to afford 0.5 g (77%) of the title compound, mp 239-241° C. ¹H NMR (CDCl₃): δ 7.55 (s, 1H), 7.40 (s, 1H), 6.73 (s, 1H), 6.67 (s, 1H), 6.47 (s, 1H), 6.44 (s, 1H), 6.24 (s, 1H), 4.63 (br, 1H), 4.38 (t, 2H, J=5.6 Hz), 3.89 (t, 2H, J=4.4 Hz), 2.35 (m, 2H), 2.11 (s, 6H), 2.06-1.84 (m, 4H), 1.56 (s, 6H), 1.30 (s, 6H). Anal. Calcd for C₂₉H₃₆N₂O₄.⅔H₂O: C, 71.28; H, 7.70; N, 5.73. Found: C, 71.10; H, 7.51; N, 5.44.

### Example 5

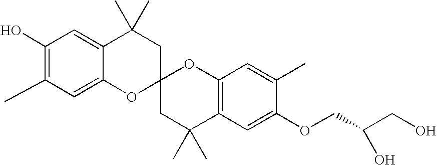

### (±)-6′-Hydroxy-6-[2(S),3-dihydroxypropoxy]-4,4,4′,4′,7,7′-hexamethyl-2,2′-spirobichroman

To (±)-6,6′-dihydroxy-4,4,4′,4′,7,7′-hexamethyl-2,2′-spirobichroman (1 g, 2.7 mmol) in 10 mL of DMF were added (R)-(−)-2,2-dimethyl-1,3-dioxolan-4-ylmethyl p-toluene-sulfonate (0.78 g, 2.7 mmol) and NaOH (0.12 g, 2.7 mmol). The mixture was irradiated in microwave at 100° C. for 15 min. The crude reaction mixture was poured into water and acidified to pH <1 with 3N HCl. Filtration afforded an off-white solid, which was dissolved in 20 mL of MeOH and 5 mL of 3N HCl. The mixture was stirred at room temperature overnight and then adjusted to pH 6 with 1N NaOH. It was extracted with ethyl acetate and the combined organic extracts were washed with brine and water. Silica gel chromatography (hexanes/ethyl acetate 1:1) gave a solid. Recrystallization from dichloromethane and hexanes gave 0.58 g (48%) of the title compound as a white solid, mp 218-220° C. ¹H NMR (acetone-d₆): δ 7.63 (s, 1H), 6.89 (s, 1H), 6.76 (s, 1H), 6.34 (s, 1H), 6.29 (s, 1H), 4.01-3.92 (m, 4H), 3.77-3.73 (m, 1H), 3.68-3.63 (m, 2H), 2.83 (s, 6H), 2.05-1.86 (m, 4H), 1.57 (s, 3H), 1.51 (s, 3H), 1.31 (s, 3H), 1.25 (s, 3H). Anal. Calcd for C₂₆H₃₄O₆.H₂O: C, 67.80; H, 7.88. Found: C, 67.74; H, 7.51.

### Example 6

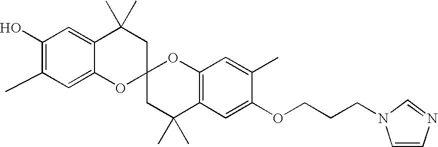

### (±)-6′-Hydroxy-6-[3-(1H-imidazol-1-yl)propoxy]-4,4,4′,4′,7,7′-hexamethyl-2,2′-spirobichroman

To (±)-6′-methanesulfonyloxy-6-(3-methanesulfonyloxypropoxy)-4,4,4′,4,7,7′-hexa-methyl-2,2′-spirobichroman (Ex. 4a, 0.5 g, 0.86 mmol) and imidazole (0.12 g, 1.72 mmol) in 25 mL of DMF at room temperature was added sodium hydride (0.10 g, 2.58 mmol, 60% dispersion in mineral oil) portionwise. The reaction mixture was stirred at room temperature for 3 h and then poured into water. It was extracted with ethyl acetate and the combined organic extracts were washed with brine and water. The organic extracts were concentrated to a residue which was dissolved in 20 mL of THF/MeOH (1:1) and 5N NaOH (3 ml) was added. The resultant mixture was heated to reflux for 5 days. The reaction mixture was acidified with 3N HCl and extracted with EtOAc. The organic extracts were combined, washed with brine, and concentrated. The residue obtained was then recrystallized from ethyl acetate and hexanes to give 0.08 g (19%) of the title compound as a light-brown solid, mp 209-211° C. ¹H NMR (CDCl₃): δ 7.51 (s, 1H), 7.08 (s, 1H), 6.94 (s, 1H), 6.75 (s, 1H), 6.66 (s, 1H), 6.48 (s, 1H), 6.44 (s, 1H), 4.21 (t, 2H, J=6.7 Hz), 3.90 (t, 2H, J=5.1 Hz), 2.21 (m, 2H), 2.11 (s, 6H), 2.06-1.84 (m, 4H), 1.56 (s, 6H), 1.31 (s, 6H). HRMS (EI) Calcd for C₂₉H₃₆N₂O₄: 476.2675 (M⁺); found 476.2674.

### Example 7

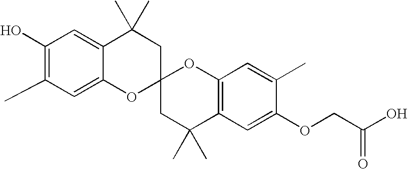

### (±)-6′-(Hydroxy-4,4,4′,4′,7,7′-hexamethyl-2,2′-spirobichroman-6-yloxy)-acetic acid

To (±)-6,6′-dihydroxy-4,4,4′,4′,7,7′-hexamethyl-2,2′-spirobichroman (1.48 g, 4.0 mmol) in 20 mL of DMF was added K₂CO₃ (1.12 g, 8 mmol). The mixture was heated to 80° C. and then bromoacetic acid ethyl ester (1.36 g, 8 mmol) was added. The mixture was stirred at 80° C. overnight, cooled to room temperature, and poured into water. The mixture was then extracted with dichloromethane, and the organic layers were combined, washed with brine, dried over MgSO₄, and concentrated to dryness. The residue obtained was dissolved in 50 mL of THF and 20 mL of 5N NaOH was added. The mixture was stirred at room temperature for 1 h, adjusted to pH=6 with 3N HCl, and extracted with dichloromethane. The organic layers were combined, washed with brine, dried over MgSO₄, and concentrated to dryness. Silica gel chromatography with 1:1 hexanes/ethyl acetate as the eluant gave 0.30 g (17%) of the title compound as a white solid, mp 227-229° C. ¹H NMR (acetone-d₆): δ 6.87 (s, 1H), 6.76 (s, 1H), 6.37 (s, 1H), 6.29 (s, 1H), 4.63 (s, 2H), 2.07 (s, 3H), 2.01 (s, 3H), 2.05-1.86 (m, 4H), 1.54 (s, 3H), 1.51 (s, 3H), 1.28 (s, 3H), 1.25 (s, 3H). Anal. Calcd for C₂₅H₃₀O₆: C, 70.40; H, 7.09. Found: C, 70.64; H, 7.23.

### Example 8

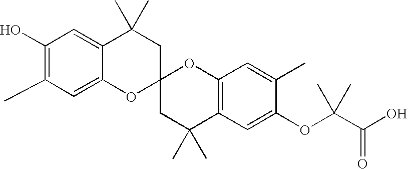

### (±)-(6′-Hydroxy-4,4,4′,4′,7,7′-hexamethyl-2,2′-spirobichroman-6-yloxy)-dimethylacetic acid

Ex. 8a. To (±)-6,6′-dihydroxy-4,4,4′,4′,7,7′-hexamethyl-2,2′-spirobichroman (18.5 g) in 350 mL of DMF was added potassium carbonate (12.8 g) and the resultant mixture was heated to 125° C. Ethyl 2-bromo-isobutyrate (13.5 g) was added and the mixture was stirred at 125° C. for 15 min. This reaction mixture was poured into 900 mL of 0.5 N HCl and subsequently extracted with ethyl acetate. The combined organic extracts were washed with 0.5 N HCl, saturated NaHCO₃ and brine. The organic layer was dried over Na₂SO₄, filtered and concentrated. Dichloromethane was added and the resulting solid was filtered off. The solution was then concentrated and subjected to chromatography (33% EtOAc/hexanes) affording 8.32 g of (±)-(6′-hydroxy-4,4,4′,4′,7,7′-hexamethyl-2,2′-spirobichroman-6-yloxy)-dimethylacetic acid ethyl ester as an off-white solid. ¹H NMR (CDCl₃): δ 6.73 (s, 1H), 6.69 (s, 1H), 6.44 (s, 1H), 6.42 (s, 1H), 4.30 (s, 1H), 4.29-4.25 (m, 2H), 2.11 (s, 3H), 2.08 (s, 3H), 1.97 (ABq, 2H, J_{AB}=13.8 Hz, Δν_{AB}=46.5 Hz), 1.96 (ABq, 2H, J_{AB}=13.8 Hz, Δν_{AB}=49.6 Hz), 1.55 (s, 9H), 1.52 (s, 3H), 1.31 (t, 3H, J=7.2 Hz), 1.31 (s, 3H), 1.26 (s, 3H).

To (±)-(6′-hydroxy-4,4,4′,4′,7,7′-hexamethyl-2,2′-spirobichroman-6-yloxy)-dimethyl-acetic acid ethyl ester (Ex. 8a, 5.87 g) in 80 mL of THF and 40 mL of EtOH was added 12.2 mL of 5N NaOH at room temperature. This mixture was heated to 75° C. for 1.5 h. The reaction mixture was concentrated and water was added. It was acidified to pH 4 and the resulting solid was isolated by filtration and washed with water. The wet cake was recrystallized from 35 mL of EtOH and 40 mL of water. The crude product (84% pure) was subjected to chromatography (0-10% MeOH/CH₂Cl₂), followed by recrystallization from 15 mL EtOH and 20 mL of water to give 1.26 g of the title compound as a white solid, mp 145-147° C. ¹H NMR (CDCl₃): δ 6.82 (s, 1H), 6.73 (s, 1H), 6.49 (s, 1H), 6.43 (s, 1H), 4.30-4.50 (br, 1H), 2.11 (s, 3H), 2.10 (s, 3H), 1.99 (ABq, 2H, J_{AB}=14.1 Hz, Δν_{AB}=46.3 Hz), 1.97 (ABq, 2H, J_{AB}=14.1 Hz, Δν_{AB}=34.3 Hz), 1.56 (s, 3H), 1.55 (s, 6H), 1.54 (s, 3H), 1.31 (s, 3H), 1.29 (s, 3H). Anal. Calcd for C₂₇H₃₄O₆.⅔H₂O: C, 69.51; H, 7.63. Found: C, 69.77; H, 7.64.

### Example 9

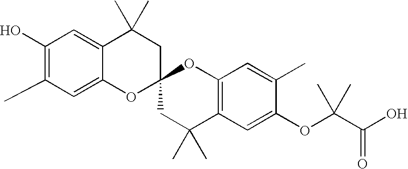

### (±)-(R)-(6′-Hydroxy-4,4,4′,4′,7,7′-hexamethyl-2,2′-spirobichroman-6-yloxy)-dimethylacetic acid

Ex. 9a. (±)-(6′-Hydroxy-4,4,4′,4′,7,7′-hexamethyl-2,2′-spirobichroman-6-yloxy)-di-methylacetic acid ethyl ester (Ex. 8a, 10 g) was separated via SFC (supercritical fluid chromatography) on a Chiralpak OD (250 mm×30 mm) column running (88/22 v/v CO₂/EtOH) isocratic with a flow rate of 180 mL/min to afford two separate enantiomers. (4 g each). Analytical chiral HPLC (Chiralpak OD-H; 250 mm×4.6 mm; 5 μM particle size; 1% EtOH in hexanes; flow rate: 1.5 mL/min; 5 μL injection; 254 nM, room temperature) showed the faster-eluting enantiomer (retention time=4 min) and slower-eluting enantiomer (retention time=5 min) to have enantiomeric purity of 99% e.e. and 98% e.e., respectively. The faster-eluting enantiomer was identified based on optical rotation, NMR and X-ray as (+)-(R)-(6′-hydroxy-4,4,4′,4′,7,7′-hexamethyl-2,2′-spirobi-chroman-6-yloxy)-dimethylacetic acid ethyl ester as a white solid, mp 166-167° C. ¹H NMR (CDCl₃): δ 6.72 (s, 1H), 6.69 (s, 1H), 6.44 (s, 1H), 6.41 (s, 1H), 4.30 (s, 1H), 4.32-4.20 (m, 2H), 2.11 (s, 3H), 2.08 (s, 3H), 1.96 (ABq, 2H, J_{AB}=13.6 Hz, Δν_{AB}=44.1 Hz), 1.95 (ABq, 2H, J_{AB}=13.6 Hz, Δν_{AB}=48.3 Hz), 1.55 (s, 9H), 1.52 (s, 3H), 1.30 (t, 3H, J=7.2 Hz), 1.30 (s, 3H), 1.26 (s, 3H). Anal. Calcd for C₂₉H₃₈O₆: C, 72.17; H, 7.94. Found: C, 72.22; H, 7.90.

To a solution of (+)-(R)-(6′-hydroxy-4,4,4′,4′,7,7′-hexamethyl-2,2′-spirobichroman-6-yloxy)-dimethylacetic acid ethyl ester (Ex. 9a, 1.03 g) in 12 mL of THF and 6 mL of EtOH at room temperature was added 2 mL of 50% aq. NaOH (Aldrich). This mixture was heated to 75° C. for 4.25 h. The reaction mixture was diluted with H₂O and washed with Et₂O. The aqueous layer was acidified to pH 1 with 6 N HCl and extracted three times with Et₂O. The ethereal extracts were dried over Na₂SO₄, filtered, and concentrated to give an orange oil. The crude oil was dissolved in 2 mL of EtOH, 3 mL of hexanes was added, and the mixture was concentrated. Concentration in the presence of hexanes and subsequent drying overnight at 45° C. under high vacuum afforded 788 mg (81%) of the title compound as an off-white foamy solid, mp 99-101° C.; positive optical rotation. ¹H NMR (CDCl₃): δ 6.84 (s, 1H), 6.76 (s, 1H), 6.52 (s, 1H), 6.45 (s, 1H), 4.32 (br s, 1H), 2.14 (s, 3H), 2.12 (s, 3H), 1.99 (ABq, 2H, J_{AB}=13.9 Hz, Δν_{AB}=48.8 Hz), 1.97 (ABq, 2H, J_{AB}=13.9 Hz, Δν_{AB}=45.8 Hz), 1.59 (s, 3H), 1.58 (s, 3H), 1.57 (s, 3H), 1.56 (s, 3H), 1.34 (s, 3H), 1.32 (s, 3H). Anal. Calcd for C₂₇H₃₄O₆.¼H₂O: C, 70.64; H, 7.58. Found: C, 70.48; H, 7.49.

### Example 10

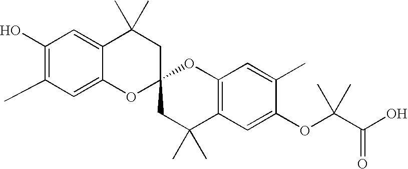

### (−)-(S)-(6′-Hydroxy-4,4,4′,4′,7,7′-hexamethyl-2,2′-spirobichroman-6-yloxy)-dimethylacetic acid

Ex. 10a. (±)-(6′-Hydroxy-4,4,4′,4′,7,7′-hexamethyl-2,2′-spirobi-chroman-6-yloxy)-di-methylacetic acid ethyl ester (Ex. 8a, 10 g) was separated via SFC (supercritical fluid chromatography) on a Chiralpak OD (250 mm×30 mm) column running (88/22 v/v CO₂/EtOH) isocratic with a flow rate of 180 mL/min to afford two separate enantiomers. (4 g each). Analytical chiral HPLC (Chiralpak OD-H; 250 mm×4.6 mm; 5 μM particle size; 1% EtOH in hexanes; flow rate: 1.5 mL/min; 5 μL injection; 254 nM, room temperature) showed the faster-eluting enantiomer (retention time=4 min) and slower-eluting enantiomer (retention time=5 min) to have enantiomeric purity of 99% e.e. and 98% e.e., respectively. The slower-eluting enantiomer was identified based on optical rotation, NMR and X-ray as (−)-(S)-(6′-hydroxy-4,4,4′,4′,7,7′-hexamethyl-2,2′-spirobi-chroman-6-yloxy)-dimethylacetic acid ethyl ester as a white solid, mp 166-167° C. ¹H NMR (CDCl₃): δ 6.72 (s, 1H), 6.69 (s, 1H), 6.44 (s, 1H), 6.41 (s, 1H), 4.30 (s, 1H), 4.32-4.20 (m, 2H), 2.11 (s, 3H), 2.08 (s, 3H), 1.96 (ABq, 2H, J_{AB}=13.6 Hz, Δν_{AB}=44.1 Hz), 1.95 (ABq, 2H, J_{AB}=13.6 Hz, Δν_{AB}=48.3 Hz), 1.55 (s, 9H), 1.52 (s, 3H), 1.30 (t, 3H, J=7.2 Hz), 1.30 (s, 3H), 1.26 (s, 3H). Anal. Calcd for C₂₉H₃₈O₆: C, 72.17; H, 7.94. Found: C, 72.09; H, 7.96.

Alternatively, to (−)-(S)-6,6′-dihydroxy-4,4,4′,4′,7,7′-hexamethyl-2,2′-spirobichroman (Ex. 27, 0.50 g, 1.36 mmol) in 1.75 ml of DMF was added t-BuOK (0.183 g, 1.63 mmol). The mixture was heated to 40° C. and stirred for 15 minutes. Ethyl 2-bromoisobutyrate (0.3 mL, 2.04 mmol) was added and the reaction mixture was stirred at 40° C. for 1 h. After cooling to room temperature, the mixture was diluted with 8 mL of a 1:1 mixture of methyl tert-butyl ether and hexanes. NaOH (8 mL, 0.5 N) was added and the mixture was stirred for 15 minutes. The layers were cut, and the organic layer was washed with 0.5 N NaOH (twice), 0.5 N HCl, and brine. The organic solution was then concentrated to dryness. Further purification by silica gel chromatography (1:4 EtOAc:hexanes) gave (−)-(S)-(6′-hydroxy-4,4,4′,4′,7,7′-hexamethyl-2,2′-spirobi-chroman-6-yloxy)-dimethylacetic acid ethyl ester (0.08 g) as a white solid, mp 165-166° C.; ¹H NMR identical to that reported above; Anal. Calcd for C₂₉H₃₈O₆: C, 72.17; H, 7.94. Found: C, 72.12; H, 7.99.

The title compound was prepared from (−)-(S)-(6′-hydroxy-4,4,4′,4′,7,7′-hexamethyl-2,2′-spirobi-chroman-6-yloxy)-dimethylacetic acid ethyl ester (Ex. 10a) in a similar manner as described in Ex. 9. The title compound was isolated as a white foam, m.p. 88-96° C. The ¹H NMR data for the title compound was identical to that of Ex. 9; in contrast to Ex. 9, the title compound had a negative optical rotation. Anal. Calcd for C₂₇H₃₄O₆: C, 71.34; H, 7.54.

Found: C, 71.38; H, 7.66.

### Example 11

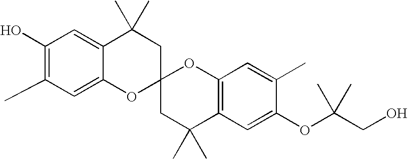

### (±)-6-(2-Hydroxy-1,1-dimethyl-ethoxy)-6′-hydroxy-4,4,4′,4′,7,7′-hexamethyl-2,2′-spirobichroman

To (±)-(6′-hydroxy-4,4,4′,4′,7,7′-hexamethyl-2,2′-spirobi-chroman-6-yloxy)-dimethyl-acetic acid ethyl ester (Ex. 8a, 0.4 g) in 5 mL of THF at room temperature was added 1.6 mL of 1 M lithium aluminum hydride in THF. This mixture was stirred at room temperature for 1.25 h and quenched with saturated Rochelle's salt solution at 0° C. The mixture was stirred at room temperature for 1 h and extracted with ethyl acetate. The organic extracts were washed with 0.5 N HCl (aq), sat NaHCO₃ (aq) and brine. The organic layer was dried over Na₂SO₄, filtered and concentrated. Further purification via silica gel chromatography with 10-40% ethyl acetate/hexanes as the eluant, followed by recrystallization from 60% EtOH/H₂O gave 0.214 g of the title compound as an off-white solid, mp 188-189° C. ¹H NMR (CDCl₃): δ 8.63 (s, 1H), 6.93 (s, 1H), 6.67 (s, 1H), 6.32 (s, 1H), 6.26 (s, 1H), 4.85 (t, 1H, J=5.1 Hz), 3.37 (d, 2H, J=5.1 Hz), 2.02-1.82 (m, 4H), 2.00 (s, 3H), 1.93 (s, 3H), 1.47 (s, 3H), 1.45 (s, 3H), 1.23 (s, 3H), 1.21 (s, 3H), 1.11 (s, 6H); Anal. Calcd for C₂₇H₃₆O₅.¼H₂O: C, 72.86; H, 8.27. Found: C, 72.89; H, 8.16. HRMS calc 440.2563 (M⁺), found 440.2564.

### Example 12

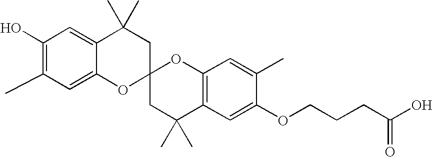

### 4-[(±)-(6′-Hydroxy-4,4,4′,4′,7,7′-hexamethyl-2,2′-spirobichroman-6-yloxy)-]butyric acid

To (±)-6,6′-dihydroxy-4,4,4′,4′,7,7′-hexamethyl-2,2′-spirobichroman (1.11 g, 3 mmol) in 10 mL of DMF was added potassium carbonate (0.84 g, 6 mmol), followed by ethyl 4-bromobutyrate (1.2 g, 6 mmol). This mixture was irradiated in microwave at 85° C. for 25 min. The reaction mixture was poured into water and the precipitate was isolated by filtration. The solid was dissolved in 50 mL of 1:1 THF/MeOH and 10 mL of 5 N NaOH was added. The resulting mixture was stirred overnight at room temperature. The reaction mixture was acidified to pH 5 with 3 N HCl. The solution was concentrated and extracted with dichloromethane. The combined organic extracts were washed with brine and water. After concentration, the crude product was purified via silica gel chromatography with 1:1 hexanes/ethyl acetate as the eluant to afford 0.35 g (25%) of the title compound as a white solid, mp 173-174° C. ¹H NMR (CDCl₃): δ 6.73 (s, 1H), 6.71 (s, 1H), 6.46 (s, 1H), 6.44 (s, 1H), 3.99 (t, 2H, J=6.0 Hz), 2.62 (t, 2H, J=7.5 Hz), 2.13 (m, 2H), 2.10 (s, 3H), 2.08 (s, 3H), 2.08-1.89 (m, 4H), 1.58 (s, 3H), 1.57 (s, 3H), 1.33 (s, 3H), 1.31 (s, 3H). Anal. Calcd for C₂₇H₃₄O₆.⅓H₂O: C, 70.10; H, 7.45. Found: C, 70.09; H, 7.49.

### Example 13

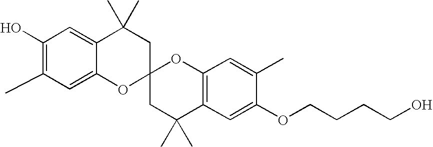

### (±)-6-(4-Hydroxybutoxy)-6′-hydroxy-4,4,4′,4′,7,7′-hexamethyl-2,2′-spirobichroman

To a solution of 4-[((±)-6′-hydroxy-4,4,4′,4′,7,7′-hexamethyl-2,2′-spirobichroman-6-yl-oxy)-]butyric acid (Ex. 12, 0.92 g, 2 mmol) in 50 mL of THF was added slowly 10 mL of BH₃ (1 M in THF, 10 mmol) at 0° C. The mixture was allowed to warm to room temperature and stirred overnight. HPLC showed that the reaction was not complete. More BH₃/THF (10 ml) was added to the mixture, and the mixture was heated to 40° C. for 3 h. The reaction was still not complete. The mixture was the quenched with MeOH, and concentrated to dryness. The residue was then dissolved in 50 mL of THF and 5 mL of lithium aluminum hydride (1 M in THF) was added at room temperature. This mixture was stirred at room temperature overnight and then quenched with a saturated Rochelle's salt solution and stirred for 1 hour. The mixture was extracted with EtOAc, and the combined organic extracts were washed with brine and water. Silica gel chromatography with NH₄OH/MeOH/dichloromethane (1:10:100) as the eluant gave 0.28 g (31%) of the title compound as a white solid, mp 235-236° C. ¹H NMR (DMSO-d₆): δ 8.62 (s, 1H), 6.80 (s, 1H), 6.66 (s, 1H), 6.31 (s, 1H), 6.23 (s, 1H), 4.39 (t, 1H, J=5.4 Hz), 3.88 (t, 2H, J=6.0 Hz), 3.42 (dt, 2H, J=5.7, 6.0 Hz), 2.02-1.82 (m, 4H), 1.97 (s, 3H), 1.92 (s, 3H), 1.75-1.65 (m, 2H), 1.60-1.50 (m, 2H), 1.49 (s, 3H), 1.45 (s, 3H), 1.25 (s, 3H), 1.20 (s, 3H). Anal. Calcd for C₂₇H₃₆O₅.¾H₂O: C, 71.42; H, 8.32. Found: C, 71.26; H, 8.08.

### Example 14

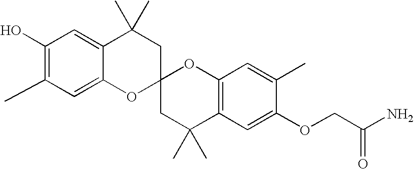

### (±)-(6′-Hydroxy-4,4,4′,4′,7,7′-hexamethyl-2,2′-spirobichroman-6-yloxy)-acetamide

To (±)-(6′-hydroxy-4,4,4′,4′,7,7′-hexamethyl-2,2′-spirobichroman-6-yloxy)-acetic acid (Ex. 7, 0.3 g) in 10 mL of DMF was added ammonium chloride (0.19 g), EDCI (0.19 g), and HOBt (0.12 g). To this mixture was added 0.25 mL of triethylamine at room temperature. This mixture was stirred at room temperature for 15 h and then at 60° C. for 2 h. The reaction mixture was diluted with ethyl acetate and washed with 0.5 N HCl (aq), saturated NaHCO₃ (aq) and brine. The organic layer was dried over Na₂SO₄, filtered and concentrated. Further purification via silica gel chromatography with 0-10% MeOH/dichloromethane, followed by recrystallization from 1:1.2 v/v EtOH/H₂O afforded 171 mg of the title compound as a solid, mp 220-221° C. ¹H NMR (d₆-DMSO): δ 8.62 (s, 1H), 7.37 (br, 1H), 7.31 (br, 1H), 6.79 (s, 1H), 6.66 (s, 1H), 6.34 (s, 1H), 6.24 (s, 1H), 4.34 (s, 2H), 2.03 (s, 3H), 2.03-1.82 (m, 4H), 1.92 (s, 3H), 1.48 (s, 3H), 1.45 (s, 3H), 1.23 (s, 3H),), 1.20 (s, 3H); C₂₅H₃₁NO₅.⅓H₂O: C, 68.63; H, 7.45; N, 3.20. Found: C, 68.46; H, 7.43; N, 3.27. HRMS Calc. 426.2280 ([M+H]⁺), found 426.2282.

### Example 15

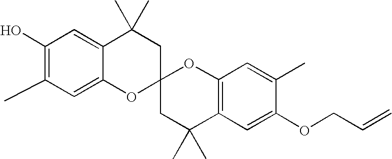

### (±)-6-Allyloxy-6′-hydroxy-4,4,4′,4′,7,7′-hexamethyl-2,2′-spirobichroman

To (±)-6,6′-dihydroxy-4,4,4′,4′,7,7′-hexamethyl-2,2′-spirobichroman (11.04 g, 30 mmol) in 300 mL of DMF was added potassium carbonate (8.4 g, 60 mmol), followed by allyl bromide (7.2 g, 60 mmol). This mixture was stirred at 85° C. for 10 minutes. The reaction mixture was poured into water and the precipitate was isolated by filtration. This crude mixture was purified by silica gel chromatography using 5:1 hexanes/ethyl acetate as the eluant to afford two products. The slower eluting component gave 4.4 g (36%) of the title compound as a white solid, mp 143-144° C. ¹H NMR (CDCl₃): δ 6.76 (s, 1H), 6.73 (s, 1H), 6.48 (s, 1H), 6.45 (s, 1H), 6.09 (ddt, 1H, J=21.9, 10.2, 5.1 Hz), 5.44 (dd, 1H, J=17.7, 1.5 Hz), 5.27 (dd, 1H, J=10.5, 1.5 Hz), 4.51 (d, 2H, J=5.1 Hz), 4.41 (s, 1H), 2.12 (s, 3H), 2.11 (s, 3H), 2.08-1.90 (m, 4H), 1.59 (s, 3H), 1.57 (s, 3H), 1.34 (s, 3H), 1.32 (s, 3H). Anal. Calcd for C₂₆H₃₂O₄: C, 76.44; H, 7.90. Found: C, 76.42; H, 7.98.

### Example 16

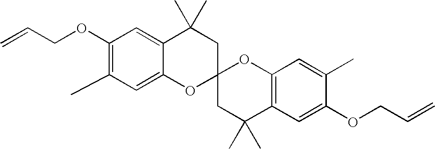

### (±)-6,6′-Bisallyloxy-4,4,4′,4′,7,7′-hexamethyl-2,2′-spirobichroman

To (±)-6,6′-dihydroxy-4,4,4′,4′,7,7′-hexamethyl-2,2′-spirobichroman (11.04 g, 30 mmol) in 300 mL of DMF was added potassium carbonate (8.4 g, 60 mmol), followed by allyl bromide (7.2 g, 60 mmol). This mixture was stirred at 85° C. for 10 minutes. The reaction mixture was poured into water and the precipitate was isolated by filtration. This crude mixture was purified by silica gel chromatography using 5:1 hexanes/ethyl acetate as the eluant to afford two products. The faster eluting component gave 4.8 g (36%) of the title compound as a white solid, mp 98-100° C. ¹H NMR (CDCl₃): δ 6.75 (s, 2H), 6.48 (s, 2H), 6.08 (ddt, 2H, J=22.8, 11.4, 5.1 Hz), 5.43 (dd, 2H, J=17.1, 1.5 Hz), 5.27 (dd, 2H, J=10.2, 1.5 Hz), 4.51 (d, 4H, J=5.1 Hz), 2.11 (s, 6H), 1.99 (ABq, 4H, J_{AB}=14.1 Hz, Δν_{AB}=34.7 Hz), 1.58 (s, 6H), 1.33 (s, 6H). Anal. Calcd for C₂₉H₃₆O₄: C, 77.64; H, 8.09. Found: C, 77.40; H, 8.05.

### Example 17

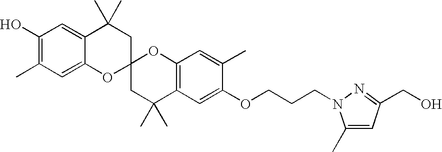

### (±)-6′-Hydroxy-6-[3-(3-hydroxymethyl-5-methyl-1H-pyrazol-1-yl)propoxy]-4,4,4′,4′,7,7′-hexamethyl-2,2′-spirobichroman

To (±)-6′-methanesulfonyloxy-6-(3-methanesulfonyloxypropoxy)-4,4,4′,4′,7,7′-hexa-methyl-2,2′-spirobichroman (Ex. 4a, 2 g, 3.4 mmol) and (5-methyl-1H-pyrazol-3-yl)-methanol (0.77 g, 6.8 mmol) in 50 mL of DMF at room temperature was added sodium hydride (0.41 g, 10.3 mmol, 60% dispersion in mineral oil) portion-wise. This reaction mixture was stirred at room temperature for 2 h and poured into water. The precipitate was isolated by filtration and re-dissolved in 60 mL of MeOH/THF (1:1 v/v). To this solution was added 5 mL of 5 N NaOH and the resulting mixture was stirred at 70° C. overnight. The reaction mixture was acidified with 3 N HCl to pH=6 and concentrated to 25 mL. This mixture was extracted with dichloromethane and the combined organic extracts were washed with brine and water. Purification by silica gel chromatography (EtOAc/Hexane, 1:2) afforded two products. The slower eluting component gave 0.22 g of the title compound as a white solid, mp 217-219° C. ¹H NMR (CDCl₃): δ 6.73 (s, 1H), 6.67 (s, 1H), 6.47 (s, 1H), 6.45 (s, 1H), 5.99 (s, 1H), 4.64 (d, 2H, J=5.2 Hz), 4.46 (s, 1H), 4.22 (t, 2H, J=7.3 Hz), 3.93-3.88 (m, 2H), 2.32-2.27 (m, 2H), 2.23 (s, 3H), 2.10 (s, 6H), 2.06-1.89 (m, 4H), 1.56 (s, 6H), 1.31 (s, 6H). HRMS (EI) Calcd for C₃₁H₄₀N₂O₅: 520.2937 (M⁺); found: 520.2932.

### Example 18

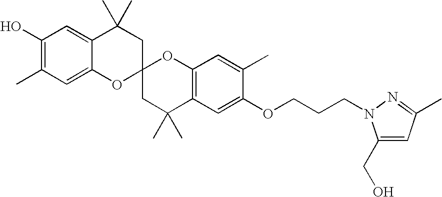

### (±)-6′-Hydroxy-6-[3-(5-hydroxymethyl-3-methyl-1H-pyrazol-1-yl)propoxy]-4,4,4′,4′,7,7′-hexamethyl-2,2′-spirobichroman

To (±)-6′-methanesulfonyloxy-6-(3-methanesulfonyloxypropoxy)-4,4,4′,4′,7,7′-hexa-methyl-2,2′-spirobichroman (Ex. 4a, 2 g, 3.4 mmol) and (5-methyl-1H-pyrazol-3-yl)-methanol (0.77 g, 6.8 mmol) in 50 mL of DMF at room temperature was added sodium hydride (0.41 g, 10.3 mmol, 60% dispersion in mineral oil) portion-wise. This reaction mixture was stirred at room temperature for 2 h and poured into water. The precipitate was isolated by filtration and re-dissolved in 60 mL of MeOH/THF (1:1 v/v). To this solution was added 5 mL of 5 N NaOH and the resulting mixture was stirred at 70° C. overnight. The reaction mixture was acidified with 3 N HCl to pH=6 and concentrated to 25 mL. This mixture was extracted with dichloromethane and the combined organic extracts were washed with brine and water. Purification by silica gel chromatography (EtOAc/Hexane, 1:2) afforded two products. The faster eluting component gave 0.095 g of the title compound as a white solid, mp 208-210° C. ¹H NMR (CDCl₃): δ 6.73 (s, 1H), 6.67 (s, 1H), 6.46 (s, 1H), 6.44 (s, 1H), 5.99 (s, 1H), 4.72-4.80 (br, 1H), 4.60 (s, 2H), 4.31 (t, 2H, J=7.2 Hz), 3.92 (t, 2H, J=5.1 Hz), 2.36-2.29 (m, 2H), 2.26 (s, 3H), 2.10 (s, 3H), 2.09 (s, 3H), 2.06-1.87 (m, 4H), 1.56 (s, 6H), 1.30 (s, 6H). HRMS (EI) Calcd for C₃₁H₄₀N₂O₅: 520.2937 (M⁺); found: 520.2940.

### Example 19

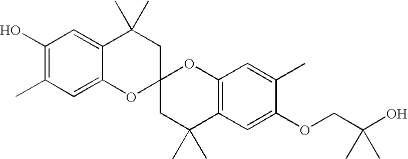

### (±)-6′-Hydroxy-6-(2-hydroxy-2-methylpropoxy)-4,4,4′,4′,7,7′-hexamethyl-2,2′-spirobichroman

Ex. 19a. A mixture of (±)-6,6′-dihydroxy-4,4,4′,4′,7,7′-hexamethyl-2,2′-spirobichroman (1.0 g, 2.71 mmol), ethyl bromoacetate (0.302 mL, 2.72 mmol) and K₂CO₃ (0.75 g, 5.43 mmol) in DMF (19 mL) was radiated under microwave at 85° C. for 10 min. After cooling to ambient temperature, the reaction mixture was poured into HCl (0.5 N). The resulting aqueous mixture was extracted with EtOAc. The combined EtOAc extracts were then washed with saturated NaHCO₃ and brine (10%), dried (Na₂SO₄), and concentrated. The residue was purified by ISCO CombiFlash system using a 40 g-size column with gradient 10-40% EtOAc/hexanes over 18 min to give (±)-(6′-hydroxy-4,4,4′,4′,7,7′-hexamethyl-2,2′-spirobichroman-6-yloxy)-acetic acid ethyl ester as a white foam (0.3 g, 24%). ¹H NMR (DMSO-d6): δ 8.62 (s, 1H), 6.76 (s, 1H), 6.66 (s, 1H), 6.35 (s, 1H), 6.24 (s, 1H), 4.70 (s, 2H), 4.13 (q, 2H, J=7.3 Hz), 2.01 (s, 3H), 1.97-1.81 (m, 4H), 1.92 (s, 3H), 1.46 (s, 3H), 1.45 (s, 3H), 1.22-1.11 (m, 9H). MS (ESI), 454 (M⁺). HRMS (ESI) Calcd. for C₂₇H₃₄O₆: 454.2355 (M⁺); found: 454.2363.

To a solution of 0.53 mL of 3.0 M methyl magnesium chloride in 1 mL of THF at room temperature was added a solution of (O)-(6′-hydroxy-4,4,4′,4′,7,7′-hexamethyl-2,2′-spirobichroman-6-yloxy)-acetic acid ethyl ester (Ex. 19a, 0.3 g) in 4 mL of THF. After stirring for 1.2 h, an additional 0.2 mL of 3.0 M methyl magnesium chloride was added to this reaction mixture. After another 50 min of stirring, the reaction mixture was carefully quenched with saturated ammonium chloride (aq.) solution. This mixture was extracted with ethyl acetate and the combined organic extracts were washed with 0.5 M HCl and saturated sodium bicarbonate solution. After drying over Na₂SO₄, filtration, and concentration, the crude material was passed through a pad of silica gel with 1:2 v/v EtOAc/hexanes. The filtrate was concentrated and recrystallized from 1:20 v/v THF/hexanes. The solid obtained was slurried in EtOH to remove residual THF. The title compound as obtained as a white solid (208 mg), mp 227-229° C. ¹H NMR (CDCl₃): δ 8.62 (s, 1H), 6.76 (s, 1H), 6.66 (s, 1H), 6.32 (s, 1H), 6.23 (s, 1H), 4.52 (s, 1H), 3.61 (s, 2H), 2.02-1.82 (m, 4H), 1.99 (s, 3H), 1.91 (s, 3H), 1.49 (s, 3H), 1.45 (s, 3H), 1.25 (s, 3H), 1.20 (s, 3H), 1.18 (s, 6H); Anal. Calcd for C₂₇H₃₆O₅.½H₂O: C, 72.13; H, 8.30. Found: C, 72.20; H, 8.26. HRMS calc 441.2641 ([M+H]⁺); found 441.2639.

### Example 20

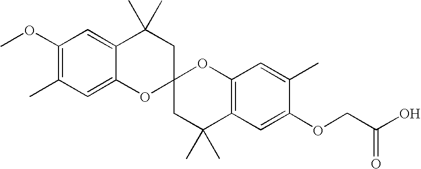

### (±)-(6′-Methoxy-4,4,4′,4′,7,7′-hexamethyl-2,2′-spirobichroman-6-yloxy)-acetic acid

To (±)-6,6′-dihydroxy-4,4,4′,4′,7,7′-hexamethyl-2,2′spirobichroman (3.7 g, 10 mmol) in 100 mL of DMF was added potassium carbonate (2.8 g, 20 mmol), followed by iodomethane (1.4 g, 10 mmol). This mixture was stirred at room temperature until HPLC showed a ratio of 26:11 of starting material and mono-methylated product. To this mixture was added ethyl bromoacetate (16.7 g, 100 mmol). The resultant mixture was stirred overnight at 85° C. The reaction mixture was poured into water and the precipitate was isolated by filtration. The crude mixture obtained was dissolved in 25 mL of THF and 10 mL of MeOH. Then 5 mL of 5 N NaOH was added. The resulting mixture was stirred 1 h at room temperature, acidified to pH=3, and extracted with dichloromethane. The combined organic extracts were washed with brine and water. After concentration, the crude product was purified via silica gel chromatography with a gradient elution from 1:1 hexanes/ethyl acetate to ethyl acetate as the eluant to afford two products. The faster eluting component gave 0.46 g (10%) of the title compound as a white solid, mp 195-197° C. ¹H NMR (CDCl₃): δ 6.73 (s, 1H), 6.72 (s, 1H), 6.50 (s, 1H), 6.46 (s, 1H), 4.63 (s, 2H), 3.81 (s, 3H), 2.15 (s, 3H), 2.08 (s, 3H), 2.08-1.90 (m, 4H), 1.60 (s, 3H), 1.58 (s, 3H), 1.35 (s, 3H), 1.32 (s, 3H). Calcd for C₂₆H₃₂O₆.¼H₂O: C, 70.17; H, 7.36. Found: C, 70.31; H, 7.30.

### Example 21

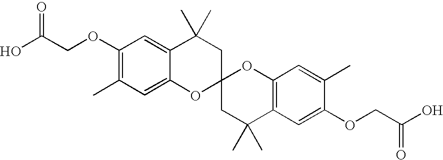

### (±)-[(4,4,4′,4′,7,7′-Hexamethyl-2,2′-spirobichroman-6,6′-diyl)bis(oxy)]-bis-acetic acid

To (±)-6,6′-dihydroxy-4,4,4′,4′,7,7′-hexamethyl-2,2-spirobichroman (3.7 g, 10 mmol) in 100 mL of DMF was added potassium carbonate (2.8 g, 20 mmol), followed by iodomethane (1.4 g, 10 mmol). This mixture was stirred at room temperature until HPLC showed a ratio of 26:11 of starting material and mono-methylated product. To this mixture was added ethyl bromoacetate (16.7 g, 100 mmol). The resultant mixture was stirred overnight at 85° C. The reaction mixture was poured into water and the precipitate was isolated by filtration. The crude mixture obtained was dissolved in 25 mL of THF and 10 mL of MeOH. Then 5 mL of 5 N NaOH was added. The resulting mixture was stirred 1 h at room temperature, acidified to pH=3, and extracted with dichloromethane. The combined organic extracts were washed with brine and water. After concentration, the crude product was purified via silica gel chromatography with a gradient eluant of 1:1 hexanes/ethyl acetate to ethyl acetate as the eluant to afford two products. The slower eluting component gave 0.55 g (11%) of the title compound as a white solid, mp>260° C. ¹H NMR (DMSO-d₆): δ 6.73 (s, 2H), 6.35 (s, 2H), 4.60 (s, 4H), 2.03-1.79 (m, 4H), 2.00 (s, 6H), 1.48 (s, 6H), 1.23 (s, 6H). Anal. Calcd for C₂₇H₃₂O₈: C, 66.93; H, 6.71. Found: C, 66.91; H, 6.71.

### Example 22

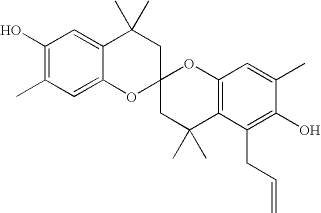

### (±)-6,6′-Dihydroxy-5-allyl-4,4,4′,4′,7,7′-hexamethyl-2,2′-spirobichroman

A solution of (±)-6-allyloxy-6′-hydroxy-4,4,4′,4′,7,7′-hexamethyl-2,2′-spirobichroman (Ex. 15, 0.7 g, 1.7 mmol) in 10 mL of N,N-dimethylaniline was irradiated in microwave at 230° C. for 30 min. The mixture was then poured into ice/water and extracted with dichlooromethane. The organic extracts were washed with 0.5 N HCl and brine, dried over MgSO₄, and concentrated. This crude product was then further purified by column chromatography (dichloromethane/hexanes, 1:1) to give 0.6 g the title compound as a white solid, mp 102-104° C. ¹H NMR (CDCl₃): δ 6.73 (s, 1H), 6.44 (s, 1H), 6.43 (s, 1H), 6.13-6.02 (m, 1H), 5.25-5.12 (m, 2H), 4.72 (s, 1H), 4.31 (s, 1H), 3.83 (ddd, 1H, J=16.9, 2.2, 2.2 Hz), 3.59 (dd, 1H, J=16.9, 5.9 Hz), 2.11 (s, 3H), 2.10 (s, 3H), 2.05-1.89 (m, 4H), 1.73 (s, 3H), 1.57 (s, 3H), 1.36 (s, 3H), 1.27 (s, 3H). Anal. Calcd for C₂₆H₃₂O₄: C, 76.44; H, 7.90. Found: C, 76.63; H, 8.29.

### Example 23

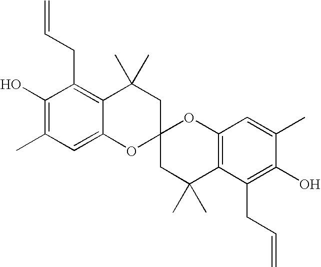

### (±)-6,6′-Dihydroxy-5,5′-diallyl-4,4,4′,4′,7,7′-hexamethyl-2,2′-spirobichroman

A solution of (±)-6,6′-bisallyloxy-4,4,4′,4′,7,7′-hexamethyl-2,2′-spirobichroman (Ex. 16, 1.0 g, 2.2 mmol) in 10 mL of N,N-dimethylaniline was irradiated in microwave at 245° C. for 15 min. The mixture was then poured into ice/water and extracted with dichloromethane. The organic extracts were washed with 0.5 N HCl and brine, dried over MgSO₄, and concentrated. This crude product was then further purified by column chromatography (dichloromethane/hexanes, 1:1) and recrystallized from dichloromethane and hexanes to give 0.12 g (12%) of the title compound as a white solid, mp 95-97° C. ¹H NMR (CDCl₃): δ 6.43 (s, 2H), 6.13-6.01 (m, 2H), 5.25-5.12 (m, 4H), 4.71 (s, 2H), 3.80 (ddd, 2H, J=16.9, 2.29, 2.2 Hz), 3.59 (dd, 2H, J=16.9, 5.9 Hz), 2.10 (s, 6H), 1.99 (s, 4H), 1.74 (s, 6H), 1.41 (s, 6H). HRMS (EI) Calcd for C₂₉H₃₆O₄ (M⁺): 448.2614; found 448.2616.

### Example 24

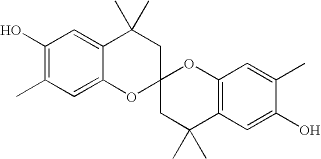

### (±)-6,6′-Dihydroxy-4,4,4′,4′,7,7′-hexamethyl-2,2′-spirobichroman

The title compound was purchased from TCI America.

### Example 25

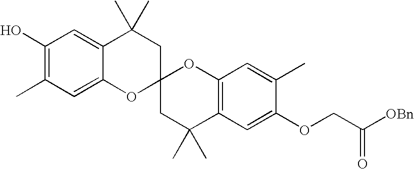

### (±)-(6′-Hydroxy-4,4,4′,4′,7,7′-hexamethyl-2,2′-spirobichroman-6-yloxy)-acetic acid Benzyl Ester

To (±)-6,6′-dihydroxy-4,4,4′,4′,7,7′-hexamethyl-2,2′-spirobichroman (2.0 g, 5.4 mmol) in 20 mL of DMF was added potassium carbonate (1.5 g, 10.8 mmol), followed by α-bromo-acetic acid benzyl ester (2.49 g, 10.8 mmol). This mixture was irradiated in microwave at 85° C. for 15 min. The reaction mixture was poured into water and the precipitate was filtered, washed with water, and dried. Silica gel chromatography (dichloromethane/hexanes, 1:4) gave 0.8 g (29%) of the title compound as a white solid, mp 60-65° C. ¹H NMR (CDCl₃): δ 7.36-7.32 (m, 5H), 6.72 (s, 1H) 6.65 (s, 1H), 6.47 (s, 1H), 6.42 (s, 1H), 5.24 (s, 2H), 4.66 (s, 1H), 4.64 (s, 2H), 2.13 (s, 3H), 2.09 (s, 3H), 1.96 (ABq, 2H, J_{ab}=24 Hz, Δν_{ab}=113 Hz), 1.95 (ABq, 2H, J_{ab}=24 Hz, Δν_{ab}=105 Hz), 1.55 (s, 3H), 1.52 (s, 3H), 1.29 (s, 3H), 1.24 (s, 3H). Anal. Calcd for C₃₂H₃₆O₆: C, 74.39; H, 7.02. Found: C, 74.19; H, 7.02.

### Example 26

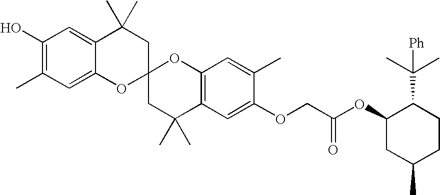

### (±)-(6′-Hydroxy-4,4,4′,4′,7,7′-hexamethyl-2,2′-spirobichroman-6-yloxy)-acetic acid 5(R)-Methyl-2(S)-(1-methyl-1-phenylethyl)-1(R)-cyclohexyl Ester

To (±)-6,6-dihydroxy-4,4,4,4′,7,7′-hexamethyl-2,2′-spirobichroman (0.37 g, 1.0 mmol) in 10 mL of DMF was added potassium carbonate (0.28 g, 2.0 mmol), followed by (1R,2S,5R)-5-methyl-2-(1-methyl-1-phenylethyl)cyclohexyl-chloroacetate (0.62 g, 2.0 mmol). This mixture was irradiated in microwave at 85° C. for 15 min. The reaction mixture was poured into water and the precipitate was filtered, washed with water, and dried. Silica gel chromatography (EtOAc/hexanes, 1:5) gave 0.23 g (19%) of the title compound as a white solid, mp 88-93° C. ¹H NMR (CDCl₃, two diasteromers): δ 7.31-7.23 (m, 8H), 7.15-7.09 (m, 2H), 6.733 (s, 1H), 6.730 (s, 1H), 6.53 (s, 1H), 6.50 (s, 1H), 6.48 (s, 2H), 6.44 (s, 1H), 6.42 (s, 1H), 4.96-4.91 (m, 2H), 4.51 (s, 1H), 4.50 (s, 1H), 3.99 (d, J=6 Hz, 1H), 3.98 (d, J=6 Hz, 1H), 3.73, (d, J=6 Hz, 1H). 3.69 (d, J=6 Hz, 1H), 2.11-2.02 (m, 20H), 1.94-1.90 (m, 4H), 1.82-1.78 (m, 2H), 1.70-1.67 (m, 2H), 1.581 (s, 3H), 1.575 (s, 3H), 1.565 (s, 3H), 1.563 (s, 3H), 1.53-1.48 (m, 2H), 1.33 (s, 12H), 1.31 (s, 12H), 1.29-1.26 (m, 4H), 1.224 (s, 3H), 1.219 (s, 3H), 1.17-1.13 (m, 2H). Anal. Calcd for C₄₁H₅₂O₆.⅔H₂O: C, 75.43; H, 8.23. Found: C, 75.19; H, 8.15.

### Example 27

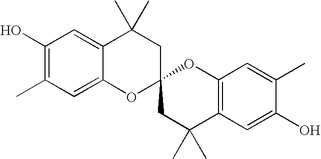

### (−)-(S)-6,6′-Dihydroxy-4,4,4′,4′,7,7′-hexamethyl-2,2′-spirobichroman

(±)-6,6′-Dihydroxy-4,4,4′,4′,7,7′-hexamethyl-2,2′-spirobichroman (from TCI America, 20 g) was separated via SFC (supercritical fluid chromatography) on a Chiralpak OD (250 mm×30 mm) column running (88/22 v/v CO₂/EtOH) isocratic with a flow rate of 180 mL/min (220 nm) to afford two separate enantiomers (9 g each). Analytical chiral HPLC (Chiralpak OD-H; 250 mm×4.6 mm; 5 μM particle size; 95:5:0.1 hexanes/EtOH/AcOH; flow rate: 2 mL/min; 5 μL injection; 254 nM, room temperature) showed the faster-eluting enantiomer (retention time=8.1 min) and slower-eluting enantiomer (retention time=10.7 min) to have enantiomeric purity of 100% e.e. and 99% e.e., respectively.

The slower-eluting enantiomer exhibited a negative optical rotation. To this enantiomer (0.50 g, 1.36 mmol) in 1.75 ml of DMF was added t-BuOK (0.183 g, 1.63 mmol). The mixture was heated to 40° C. and stirred for 15 minutes. Ethyl 2-bromoisobutyrate (0.3 mL, 2.04 mmol) was added and the reaction mixture was stirred at 40° C. for 1 hour. After cooling to room temperature, the mixture was diluted with 8 mL of a 1:1 mixture of methyl tert-butyl ether and hexanes. NaOH (8 mL, 0.5 N) was added and the mixture was stirred for 15 minutes. The layers were cut, and the organic layer was washed with 0.5 N NaOH (twice), 0.5 N HCl, and brine. The organic solution was then concentrated to dryness. Further purification by silica gel chromatography (1:4 EtOAc:hexanes) gave 0.08 g (12%) of an enantiomer of (6′-hydroxy-4,4,4′,4′,7,7′-hexamethyl-2,2′-spirobi-chroman-6-yloxy)-dimethylacetic acid ethyl ester as a white solid, mp 165-166° C. ¹H NMR (300 MHz, CDCl₃): δ 6.73 (s, 1H) 6.69 (s, 1H), 6.44 (s, 1H), 6.42 (s, 1H), 4.33 (s, 1H), 4.29-4.24 (m, 2H), 2.11 (s, 3H), 2.08 (s, 3H), 1.962 (ABq, 2H, J_{AB}=13.9 Hz, Δν_{AB}=44.3 Hz), 1.958 (ABq, 2H, J_{AB}=13.9 Hz, Δν_{AB}=48.1 Hz), 1.56 (s, 3H), 1.55 (s, 3H), 1.524 (s, 3H), 1.519 (s, 3H), 1.31 (s, 3H), 1.31 (t, 3H, J=7.04 Hz), 1.26 (s, 3H). Anal. Calcd for C₂₉H₃₈O₆: C, 72.17; H, 7.94. Found: C, 72.12; H, 7.99. This enantiomer was identified as (−)-(S)-(6′-hydroxy-4,4,4′,4′,7,7′-hexamethyl-2,2′-spirobi-chroman-6-yloxy)-dimethylacetic acid ethyl ester based on the comparison with an authentic sample (Ex. 10a) obtained by chiral HPLC (ChiralPak IA 0.46 cm×25 cm; hexanes/EtOH/TFA (95/5/0.1); 1 mL/min; 10 μl injection; 25° C.; 280 nm): R_{T}=7.05 min. Therefore, the title compound (the slower-eluting enantiomer) was determined as (−)-(S)-6,6′-dihydroxy-4,4,4′,4′,7,7′-hexamethyl-2,2′-spirobichroman.

### Example 28

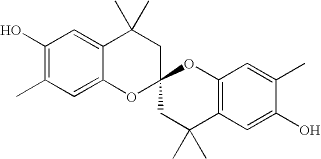

### (+)-(R)-6,6′-Dihydroxy-4,4,4′,4′,7,7′-hexamethyl-2,2′-spirobichroman

(±)-6,6′-Dihydroxy-4,4,4′,4′, 7,7′-hexamethyl-2,2′-spirobichroman (from TCI America, 20 g) was separated via SFC (supercritical fluid chromatography) on a Chiralpak OD (250 mm×30 mm) column running (88/22 v/v CO₂/EtOH) isocratic with a flow rate of 180 mL/min (220 nm) to afford two separate enantiomers (9 g each). Analytical chiral HPLC (Chiralpak OD-H; 250 mm×4.6 mm; 5 μM particle size; 95:5:0.1 hexanes/EtOH/AcOH; flow rate: 2 mL/min; 5 μL injection; 254 nM, room temperature) showed the faster-eluting enantiomer (retention time=8.1 min) and slower-eluting enantiomer (retention time=10.7 min) to have enantiomeric purity of 100% e.e. and 99% e.e., respectively. The faster-eluting enantiomer (a white powder) showed a positive optical rotation. Based on the correlation study on the slower-eluting enantiomer (Ex. 27), the faster-eluting enantiomer was determined as (+)-(R)-6,6′-dihydroxy-4,4,4′,4′,7,7′-hexamethyl-2,2′-spirobichroman, white solid, mp 93-99° C.; ¹H NMR identical to Ex. 27.

### Example 29

### In Vitro IL-6 and IL-8 Assays

Compounds at dosing concentrations (with 0.2% DMSO vehicle) were incubated in EGM2MV cell media at 37° C. for overnight, added to 96-well plates seeded with confluent human pulmonary artery endothelial cells (HPAEC) for 20 hrs at 37° C., and then replaced with fresh dosing media containing 2 ng/ml tumor necrosis factor alpha for additional 4 hrs at 37° C. Afterwards, media was removed and used in ELISA assay to quantify the amount of IL-6 and IL-8 secreted by the cells using R&D Systems Duoset ELISA kits. Data of compounds from this assay are reported in Table 1.

### Example 30

### Protocol for Testing Compounds in a Mouse Model of Asthma

Male 5-6 week old Balb/CJ mice were obtained from Jackson Laboratories (Bar Harbor, Me.). All experimental animals were used in accordance to Institutional Animal Care and Use Committee of AtheroGenics, Inc. Mice were sensitized by administering an intraperitoneal injection of 20 μg of ovalbumin (Calbiochem, La Jolla, Calif.) adsorbed in 2 mg of alum (Imject Alum; Pierce, Rockford, Ill.) on day 0 and 14. A group of mice received saline and served as negative control animals. The mice were challenged by aerosol exposure to ovalbumin (1% [wt/vol]) for 25 minutes on 3 consecutive days (days 28, 29, and 30) in a plexiglas exposure chamber coupled to an Aeroneb nebulizer (Buxco Electronics, Wilmington, N.C.). Experimental compounds were dissolved in Glycofurol/PEG 300/Tween (35%/55%/10%) (Sigma-aldrich; Milwaukee, Wis.). Animals were dosed orally with either test compound or vehicle (6 ml/kg dosing volume) on days 26-32 of study. Compound or vehicle was administered 2 hour before the aerosol challenge on days 28, 29, and 30 and 1 hour before airway reactivity measurement on day 32.

Methacholine-induced airway reactivity was assessed on day 32. Methacholine was administered in increasing concentrations (0.375, 0.77, 1.5, 3, 6, 12, 25, and 50 mg/ml) to unrestrained mice. Increases in airway resistance to Methacholine were determined as enhanced pause, (Penh) values, during and after the exposure (6-minute total analysis time). Mice were then humanely euthanized with an overdose of ketamine/xylazine and plasma samples collected for determination of drug levels. The data is presented as the % inhibition of the PenH vs McH dose AUC compared with the vehicle control.

Data analysis was conducting using the software package, JMP (SAS Institute Inc; Cary, N.C.). The Dunnett's multiple comparison test was used to compare treatment group means to the vehicle control group. P values of less than 0.05 were considered statistically significant. The data (Table 1) are presented as the % inhibition of the PenH vs McH dose AUC compared with the vehicle control.

TABLE 1

| | | Inhibition | Inhibition | Reduction |
|---|---|---|---|---|
| | | (%) of IL- | (%) of IL- | of Penh |
| Example | | 8 at 10 | 6 at 10 | AUC at 50 |
| No. | Compound Structure | microM | microM | mg/kg p.o. |
| 1 | 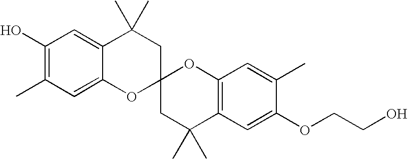 | 0 | 20 | 3 |
| 2 | 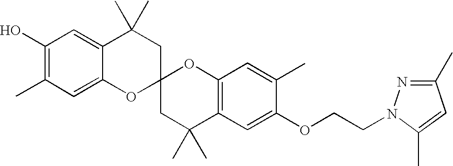 | 12 | 9 | 3 |
| 3 | 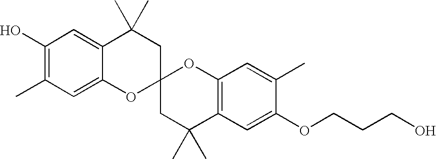 | 13 | 53 | 2 |
| 4 | 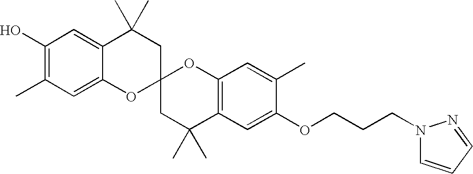 | 0 | 44 | 3 |
| 5 | 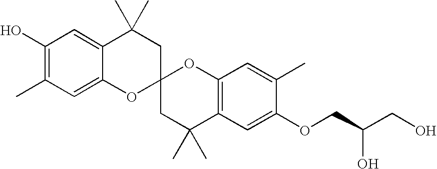 | 11 | 3 | 2 |
| 6 | 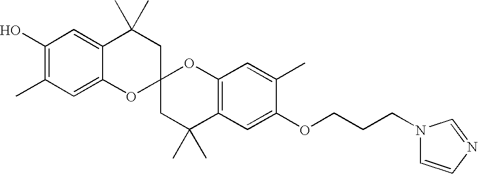 | 5 | 24 | not tested |
| 7 | 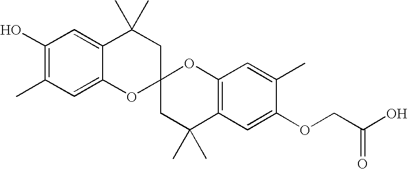 | 0 | 12 | 1 |
| 8 | 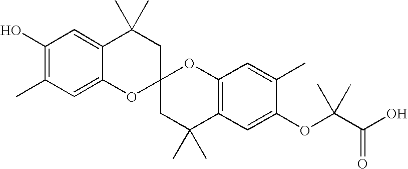 | 25 | 42 | 1 |
| 9 | 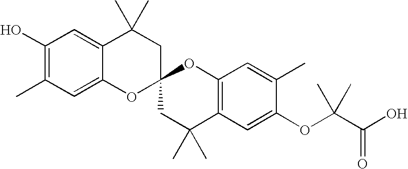 | not tested | not tested | 1 |
| 10 | 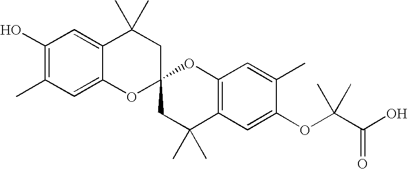 | not tested | not tested | 3 |
| 11 | 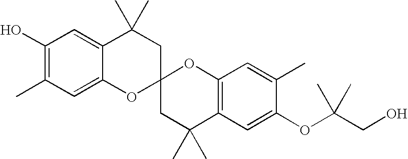 | not tested | not tested | 3 |
| 12 | 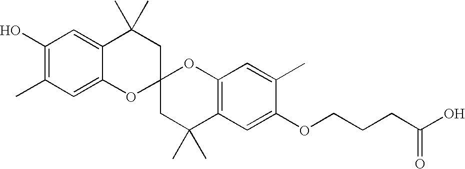 | 0 | 0 | 3 |
| 13 | 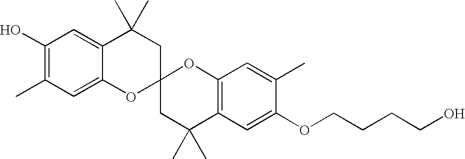 | not tested | not tested | not tested |
| 14 | 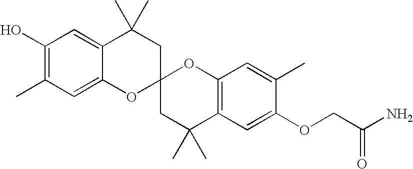 | not tested | not tested | 3 |
| 15 | 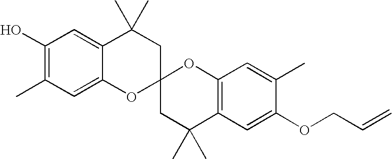 | not tested | not tested | not tested |
| 16 | 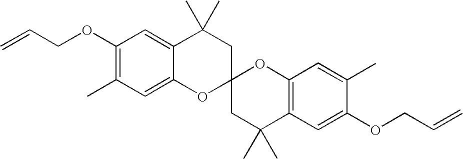 | not tested | not tested | not tested |
| 17 | 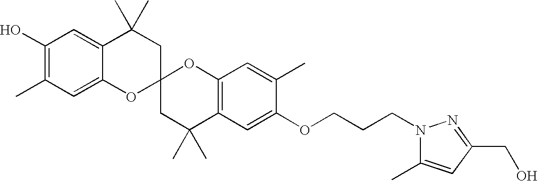 | not tested | not tested | 2 |
| 18 | 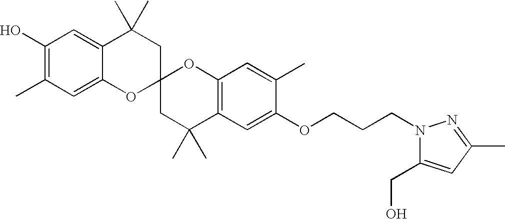 | 5 | 41 | not tested |
| 19 | 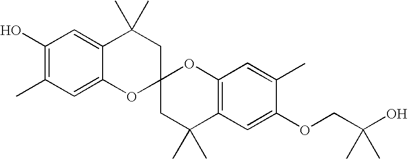 | not tested | not tested | 3 |
| 20 | 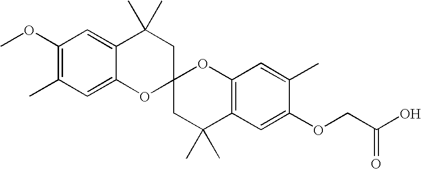 | not tested | not tested | 3 |
| 21 | 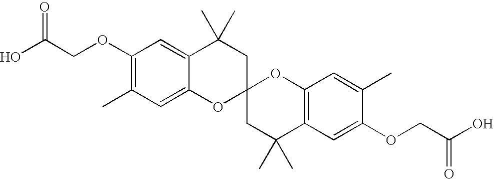 | 19 | 25 | 2 |
| 22 | 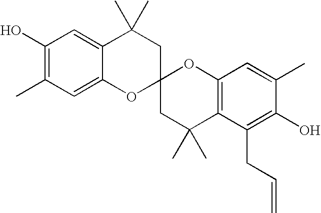 | not tested | not tested | 3 |
| 23 | 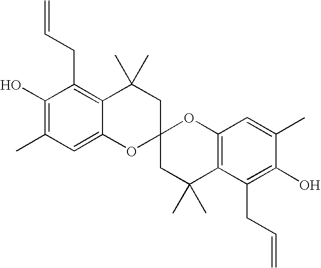 | not tested | not tested | 3 |
| 24 | 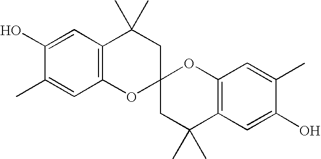 | 51 | 65 | 2 |
| 25 | 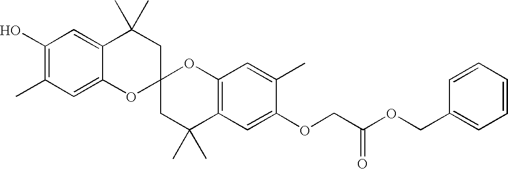 | not tested | not tested | not tested |
| 26 | 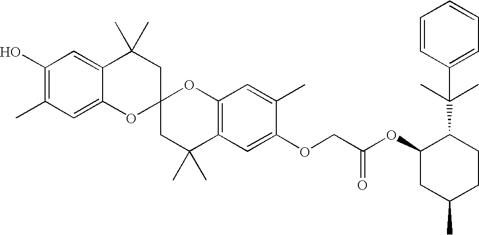 | not tested | not tested | not tested |
| 27 | 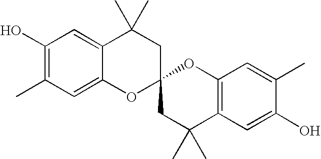 | not tested | not tested | 1 |
| 28 | 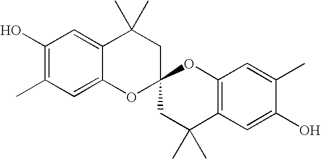 | not tested | not tested | 2 |

1: >40% and statistically significant
2: 20-40% and either statistically significant or not
3: <20% (no significant activity)

We claim:

1. A compound of Formula IV: 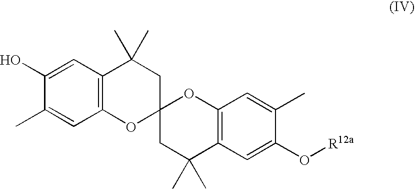
or a pharmaceutically acceptable salt or prodrug thereof, wherein:
R^{12a} is selected from the group consisting of carboxy-C₁-C₆ straight alkyl, carboxy-C₃-C₆ branched alkyl, carboxy-C₂-C₆ alkenyl, carboxy-C₃-C₈ cyclic alkyl, (C(O)NHR^{13a})—C₁-C₆ straight alkyl, (C(O)NHR^{13a})—C₃-C₆ branched alkyl, (C(O)NHR^{13a})—C₂-C₆ alkenyl, (C(O)NHR^{13a})—C₃-C₈ cyclic alkyl, heteroaralkyl, heterocyclicalkyl, and aralkyl, wherein all may be optionally substituted by one or more independently selected from the group consisting of halo, alkyl, lower alkyl, alkenyl, cycloalkyl, acyl, hydroxy, hydroxyalkyl, heterocyclic, heteroaryl, aryl, amino, aminoalkyl, —NR^{14a}R^{15a}, oxo, cyano, carboxy, carboxyalkyl, alkoxycarbonyl, OR^{16a}, —C(O)R^{16a}, —C(O)—NH₂, —C(O)—N(H)R^{14a}, —C(O)—N(H)OR^{14a}—C(O)NR^{14a}R^{5a}, NR^{15a}C(O)R^{14a}—NR^{15a}C(O)NR^{14a}R^{15a}, —OC(O)NR^{14a}R^{15a}, —NR^{15a}C(O)OR^{16a}, —S(O)ₙ—R^{16a}, —S(O)₂—NH₂, —S(O)₂—N(H)R^{14a} and —S(O)₂—NR^{14a}R^{15a}; or
R^{12a} is selected from the group consisting of hydroxy-C₁-C₆ straight alkyl, hydroxy-C₃-C₆ branched alkyl, hydroxy-C₂-C₆ alkenyl, and hydroxy-C₃-C₈ cyclic alkyl, wherein all may be optionally substituted by one or more independently selected from the group consisting of halo, alkyl, lower alkyl, alkenyl, cycloalkyl, acyl, hydroxy, hydroxyalkyl, heterocyclic, heteroaryl, aryl, amino, aminoalkyl, —NR^{14a}R^{15a}, OXO, cyano, carboxy, carboxyalkyl, alkoxycarbonyl, —OR^{16a}, —C(O)R^{16a}, —C(O)—NH₂, —C(O)—N(H)R^{14a}, —C(O)—N(H)OR^{14a}—C(O)—NR^{14a}R^{15a}, —NR^{15a}C(O)R^{14a}, —NR^{15a}C(O)NR^{14a}R^{15a}, —OC(O)NR^{14a}R^{15a}, —NR^{15a}C(O)OR^{16a}, —S(O)ₙ—R^{16a}, —S(O)₂—NH₂, —S(O)₂—N(H)R^{14a} and —S(O)₂—NR^{14a}R^{15a};
each n is independently 0, 1, or 2;
R^{13a} is selected from the group consisting of hydrogen, hydroxy, C₁-C₆ straight alkyl, C₁-C₆ branched alkyl, C₂-C₆ alkenyl, C₃-C₈ cyclic alkyl, heterocyclic, heteroaryl and aryl, wherein all may be optionally substituted by one or more independently selected from the group consisting of halo, alkyl, lower alkyl, acyl, oxo, hydroxy, hydroxyalkyl, heterocyclic, heteroaryl, cyano, amino, aminoalkyl, and carboxy;
R^{14a} and R^{15a} are independently selected from the group consisting of hydrogen, C₁-C₆ straight alkyl, C₁-C₆ branched alkyl, C₂-C₆ alkenyl, C₃-C₈ cyclic alkyl, aryl, heteroaryl, heterocycle, and acyl, wherein all may be optionally substituted by one or more independently selected from the group consisting of halo, alkyl, lower alkyl, alkenyl, cycloalkyl, acyl, hydroxy, hydroxyalkyl, heterocyclic, heteroaryl, aryl, amino, aminoalkyl, oxo, cyano, carboxy, carboxyalkyl, alkoxycarbonyl, and —OR^{16a};
R^{14a} and R^{15a} taken together may form a 4- to 12-membered monocyclic, bicyclic, tricyclic or benzofused ring;
R^{16a} is selected from the group consisting of C₁-C₆ straight alkyl, C₁-C₆ branched alkyl, C₂-C₆ alkenyl, C₃-C₈ cyclic alkyl, heterocyclic, heteroaryl and aryl, wherein all may be substituted by one or more independently selected from the group consisting of halo, alkyl, lower alkyl, acyl, oxo, hydroxy, hydroxyalkyl, heterocyclic, heteroaryl, aryl, amino, aminoalkyl, cyano, and carboxy.

2. The compound of claim 1 in the form of an isolated enantiomer.

3. The compound of claim 1 wherein R^{12a} includes a chiral center.

4. The compound of claim 3 in the form of an isolated diastereomer.

5. The compound of claim 1 wherein R^{12a} is selected from the group consisting of carboxy-C₁-C₆ straight alkyl, carboxy-C₃-C₆ branched alkyl, carboxy-C₃-C₈ cyclic alkyl, (C(O)NHR^{13a})—C₁-C₆ straight alkyl, (C(O)NHR^{13a})—C₃-C₆ branched alkyl, (C(O)NHR^{13a})—C₃-C₈ cyclic alkyl, heteroaralkyl, and heterocyclicalkyl, wherein all may be optionally substituted by one or more independently selected from the group consisting of halo, lower alkyl, cycloalkyl, acyl, hydroxy, hydroxyalkyl, heterocyclic, heteroaryl, amino, aminoalkyl, —NR^{14a}R^{15a}, oxo, cyano, carboxy, carboxyalkyl, alkoxycarbonyl, —OR^{16a}, —C(O)R^{16a}, —C(O)—NH₂, —C(O)—N(H)R^{14a}, —C(O)—N(H)OR^{14a}, C(O)—NR^{14a}R^{15a} and —NR^{15a}C(O)R^{14a}.

6. The compound of claim 1 wherein R^{12a} is selected from the group consisting of hydroxy-C₁-C₆ straight alkyl, hydroxy-C₃-C₆ branched alkyl, hydroxy-C₂-C₆ alkenyl, hydroxy-C₃-C₈ cyclic alkyl, wherein all may be optionally substituted by one or more independently selected from the group consisting of halo, lower alkyl, cycloalkyl, acyl, hydroxy, hydroxyalkyl, heterocyclic, heteroaryl, amino, aminoalkyl, —NR^{14a}R^{15a}, oxo, cyano, carboxy, carboxyalkyl, alkoxycarbonyl, —OR^{16a}, —C(O)R^{16a}, —C(O)—NH₂, —C(O)—N(H)R^{14a}, C(O)—N(H)OR^{14a}, —C(O)—NR^{14a}R^{15a} and NR^{15a}C(O)R^{14a}.

7. The compound of claim 1 wherein R^{12a} is selected from the group consisting of carboxy-C₁-C₄ straight alkyl and carboxy-C₃-C₆ branched alkyl, wherein all may be optionally substituted by one or more independently selected from the group consisting of halo, lower alkyl, acyl, hydroxy, hydroxyalkyl, heterocyclic, heteroaryl, and amino.

8. The compound of claim 1 wherein R^{12a} is selected from the group consisting of hydroxy-C₁-C₆ straight alkyl, hydroxy-C₃-C₆ branched alkyl, hydroxy-C₂-C₆ alkenyl, hydroxy-C₃-C₈ cyclic alkyl, wherein all may be optionally substituted by one or more independently selected from the group consisting of halo, lower alkyl, acyl, hydroxy, hydroxyalkyl, heterocyclic, heteroaryl, amino, aminoalkyl, oxo, cyano, and alkoxycarbonyl.

9. The compound of claim 1 wherein the compound is 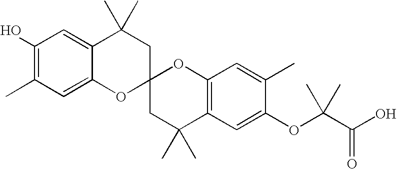
or an enantiomer thereof, or a pharmaceutically acceptable salt or prodrug thereof.

10. The compound of claim 1 wherein the compound is: 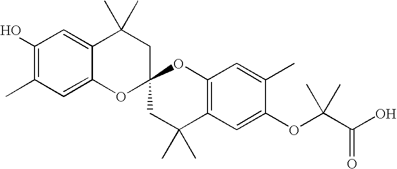
or a pharmaceutically acceptable salt or prodrug thereof.

11. The compound of claim 1 wherein the compound is 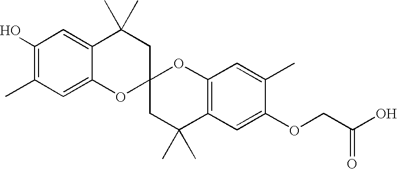
or an enantiomer thereof, or a pharmaceutically acceptable salt or prodrug thereof.

12. A pharmaceutical composition of Formula I 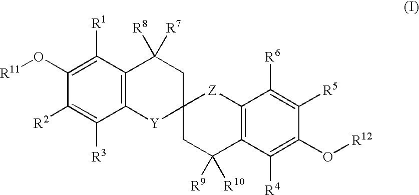
or a pharmaceutically acceptable salt, ester or prodrug thereof, and a pharmaceutically acceptable carrier wherein:
Y and Z are independently O, S(O)_{q}, Se(O)_{q} or N(R¹³);
each q is independently 0, 1 or 2;
R¹, R², R³, R⁴, R⁵ and R⁶ are independently selected from the group consisting of hydrogen, halo, C₁-C₆ straight alkyl, C₁-C₆ branched alkyl, C₂-C₆ alkenyl, C₃-C₈ cyclic alkyl, aryl and —OR⁴, wherein all may be optionally substituted by a hydroxy group;
R⁷, R⁸, R⁹ and R¹⁰ are independently selected from the group consisting of hydrogen, halo, C₁-C₆ straight alkyl, C₁-C₆ branched alkyl, and C₃-C₈ cyclic alkyl, wherein all may be optionally substituted by a hydroxy group;
R¹¹ and R¹² are independently selected from the group consisting of hydrogen, C₁-C₆ straight alkyl, C₁-C₆ branched alkyl, C₂-C₆ alkenyl, C₃-C₈ cyclic alkyl, and acyl, wherein all may be optionally substituted by one or more independently selected from the group consisting of halo, alkyl, lower alkyl, alkenyl, cycloalkyl, acyl, hydroxy, hydroxyalkyl, carboxy, alkoxy, heterocyclic, heteroaryl, aryl, amino, aminoalkyl, —NR¹⁴R¹⁵, oxo, cyano, alkoxycarbonyl, —OR¹⁶, —C(O)R¹⁶, —C(O)—NH₂, —C(O)—N(H)R¹⁴, —C(O)—N(H)OR¹⁴, —C(O)—NR¹⁴R¹⁵, —NR¹⁵C(O)R¹⁴, —NR¹⁵C(O)NR¹⁴R¹⁵, —OC(O)NR¹⁴R¹⁵, —NR¹⁵C(O)OR¹⁶, —S(O)ₙ—R¹⁶, —S(O)₂—NH₂, —S(O)₂—N(H)R¹⁴ and —S(O)₂—NR¹⁴R¹⁵;
or R¹¹ and R¹² are independently selected from the group consisting of hydrogen, aryl, heteroaryl and heterocycle, wherein all may be optionally substituted by one or more independently selected from the group consisting of halo, alkyl, lower alkyl, alkenyl, cycloalkyl, acyl, hydroxy, hydroxyalkyl, alkoxy, heterocyclic, heteroaryl, aryl, amino, aminoalkyl, —NR¹⁴R¹⁵, oxo, cyano, carboxy, carboxyalkyl, alkoxycarbonyl, —OR¹⁶, —C(O)R¹⁶, —C(O)—NH₂, —C(O)—N(H)R¹⁴, —C(O)—N(H)OR¹⁴, —C(O)—NR¹⁴R¹⁵, —NR¹⁵C(O)R¹⁴, —NR¹⁵C(O)NR¹⁴R¹⁵, —OC(O)NR¹⁴R¹⁵, —NR¹⁵C(O)OR¹⁶, —S(O)ₙ—R¹⁶, —S(O)₂—NH₂, —S(O)₂—N(H)R¹⁴ and —S(O)₂—NR¹⁴R¹⁵;
each n is independently 0, 1, or 2;
R¹³ is independently selected from the group consisting of hydrogen, hydroxy, C₁-C₆ straight alkyl, C₁-C₆ branched alkyl, C₂-C₆ alkenyl, C₃-C₈ cyclic alkyl, heterocyclic, heteroaryl and aryl, wherein all may be optionally substituted by one or more independently selected from the group consisting of halo, alkyl, lower alkyl, acyl, oxo, hydroxy, hydroxyalkyl, alkoxy, heterocyclic, heteroaryl, cyano, amino, aminoalkyl, and carboxy;
R¹⁴ and R¹⁵ are independently selected from the group consisting of hydrogen, C₁-C₆ straight alkyl, C₁-C₆ branched alkyl, C₂-C₆ alkenyl, C₃-C₈ cyclic alkyl, aryl, heteroaryl, heterocycle, and acyl, wherein all may be optionally substituted by one or more independently selected from the group consisting of halo, alkyl, lower alkyl, alkenyl, cycloalkyl, acyl, hydroxy, hydroxyalkyl, alkoxy heterocyclic, heteroaryl, aryl, amino, aminoalkyl, oxo, cyano, carboxy, carboxyalkyl, alkoxycarbonyl, and —OR¹⁶;
R¹⁴ and R¹⁵ taken together may form a 4- to 12-membered monocyclic, bicyclic, tricyclic or benzofused ring;
R¹⁶ is independently selected from the group consisting of C₁-C₆ straight alkyl, C₁-C₆ branched alkyl, C₂-C₆ alkenyl, C₃-C₈ cyclic alkyl, heterocyclic, heteroaryl and aryl, wherein all may be substituted by one or more independently selected from the group consisting of halo, alkyl, lower alkyl, acyl, oxo, hydroxy, hydroxyalkyl, heterocyclic, heteroaryl, aryl, amino, aminoalkyl, cyano, and carboxy;
with the proviso, that when R¹¹ and R¹² are heteroaryl, R¹¹ and R¹² cannot be 2-furyl.

13. The pharamaceutical composition of claim 12 wherein at least one of R¹¹ and R¹² is not hydrogen.

14. The pharamaceutical composition of claim 12, wherein the compound is of Formula II: 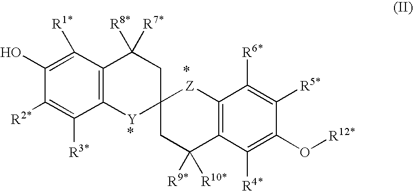
or a pharmaceutically acceptable salt, ester or prodrug thereof, wherein:
Y* and Z* are independently O, S(O)_{q}, Se(O)_{q} or N(R^{13*});
each q is independently 0, 1 or 2;
R^{1*}, R^{2*}, R^{3*}, R^{4*}, R^{15*} and R^{6*} are independently selected from the group consisting of hydrogen, halo, C₁-C₆ straight alkyl, C₁-C₆ branched alkyl, C₂-C₆ alkenyl, C₃-C₈ cyclic alkyl, aryl and —OR^{14*}, wherein all may be optionally substituted by a hydroxy group;
R^{7*}, R^{8*}, R^{9*} and R^{10*} are independently selected from the group consisting of hydrogen, halo, C₁-C₆ straight alkyl, C₁-C₆ branched alkyl, and C₃-C₈ cyclic alkyl, wherein all may be optionally substituted by a hydroxy group;
R^{12*} is selected from the group consisting of C₁-C₆ straight alkyl, hydroxy-C₁-C₆ straight alkyl, polyhydroxy-C₁-C₆ straight alkyl, carboxy-C₁-C₆ straight alkyl, carboxy-C₃-C₆ branched alkyl, carboxy-C₂-C₆ alkenyl, carboxy-C₃-C₈ cyclic alkyl, (C(O)NHR^{13*})—C₁-C₆ straight alkyl, (C(O)NHR^{13*})—C₃-C₆ branched alkyl, (C(O)NHR^{13*})—C₂-C₆ alkenyl, (C(O)NHR^{13*})—C₃-C₈ cyclic alkyl, heteroaralkyl, heterocyclicalkyl, and aralkyl, wherein all may be optionally substituted by one or more independently selected from the group consisting of halo, alkyl, lower alkyl, alkenyl, cycloalkyl, acyl, hydroxy, hydroxyalkyl, heterocyclic, heteroaryl, aryl, amino, aminoalkyl, —NR^{14*}R^{15*}, oxo, cyano, carboxy, carboxyalkyl, alkoxycarbonyl, —OR^{16*}, —C(O)R^{16*}, —C(O)—NH₂, —C(O)—N(H)R^{14*}, C(O)—N(H)OR^{14*}, C(O)NR^{14*}R^{15*}, —NR^{15*}C(O)R^{14*}, —NR^{15*}C(O)NR^{14*}R^{15*}, —OC(O)NR^{14*}R^{15*}, NR^{15*}C(O)OR^{16*}, —S(O)ₙ—R^{16*}, —S(O)₂—NH₂, —S(O)₂—N(H)R^{14*} and —S(O)₂—NR^{14*}R^{15*};
each n is independently 0, 1, or 2;
R^{13*} is selected from the group consisting of hydrogen, hydroxy, C₁-C₆ straight alkyl, C₁-C₆ branched alkyl, C₂-C₆ alkenyl, C₃-C₈ cyclic alkyl, heterocyclic, heteroaryl and aryl, wherein all may be optionally substituted by one or more independently selected from the group consisting of halo, alkyl, lower alkyl, acyl, oxo, hydroxy, hydroxyalkyl, heterocyclic, heteroaryl, cyano, amino, aminoalkyl, and carboxy;
R^{14*} and R^{15*} are independently selected from the group consisting of hydrogen, C₁-C₆ straight alkyl, C₁-C₆ branched alkyl, C₂-C₆ alkenyl, C₃-C₈ cyclic alkyl, aryl, heteroaryl, heterocycle, and acyl, wherein all may be optionally substituted by one or more independently selected from the group consisting of halo, alkyl, lower alkyl, alkenyl, cycloalkyl, acyl, hydroxy, hydroxyalkyl, heterocyclic, heteroaryl, aryl, amino, aminoalkyl, oxo, cyano, carboxy, carboxyalkyl, alkoxycarbonyl, and —OR^{16*};
R^{14*} and R^{15*} taken together may form a 4- to 12-membered monocyclic, bicyclic, tricyclic or benzofused ring;
R^{16*} is independently selected from the group consisting of C₁-C₆ straight alkyl, C₁-C₆ branched alkyl, C₂-C₆ alkenyl, C₃-C₈ cyclic alkyl, heterocyclic, heteroaryl and aryl, wherein all may be substituted by one or more independently selected from the group consisting of halo, alkyl, lower alkyl, acyl, oxo, hydroxy, hydroxyalkyl, heterocyclic, heteroaryl, aryl, amino, aminoalkyl, cyano, and carboxy.

15. The pharamaceutical composition of claim 12, wherein the compound is of Formula III: 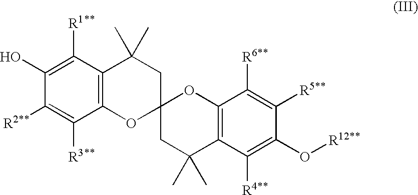
or a pharmaceutically acceptable salt, ester or prodrug thereof wherein:
R^{1**}, R^{2**}, R^{3**}, R^{4**}, R^{15**} and R^{6**} are independently selected from the group consisting of hydrogen, halo, C₁-C₄ straight alkyl, C₁-C₄ branched alkyl, C₂-C₆ alkenyl, aryl and —OR⁴;
R^{12**}is selected from the group consisting of C₁-C₆ straight alkyl, hydroxy-C₁-C₆ straight alkyl, polyhydroxy-C₁-C₆ straight alkyl, carboxy-C₁-C₆ straight alkyl, carboxy-C₃-C₆ branched alkyl, carboxy-C₂-C₆ alkenyl, carboxy-C₃-C₈ cyclic alkyl, (C(O)NHR^{13**})—C₁-C₆ straight alkyl, (C(O)NHR^{13**})—C₃-C₆ branched alkyl, (C(O)NHR^{13**})—C₂-C₆ alkenyl, (C(O)NHR^{13**})—C₃-C₈ cyclic alkyl, heteroaralkyl, heterocyclicalkyl, and aralkyl, wherein all may be optionally substituted by one or more independently selected from the group consisting of halo, alkyl, lower alkyl, alkenyl, cycloalkyl, acyl, hydroxy, hydroxyalkyl, heterocyclic, heteroaryl, aryl, amino, aminoalkyl, NR^{14**}R^{15**}, oxo, cyano, carboxy, carboxyalkyl, alkoxycarbonyl, —OR^{16**}, —C(O)R^{16**}, —C(O)—NH₂, —C(O)—N(H)R^{14**}, —C(O)—N(H)OR^{14**}, —C(O)—NR^{14**}R^{15**}, NR^{15**}C(O)R^{14**}, —NR^{15**}C(O)NR^{14**}R^{15**}, OC(O)NR^{14**}R^{15**}, —NR^{15**}C(O)OR^{16**}, —S(O)ₙ—R^{16**}, —S(O)₂—NH₂, —S(O)₂—N(H)R^{14**}and —S(O)₂NR^{14**}R^{15**};
each n is independently 0, 1, or 2;
R^{13**}is selected from the group consisting of hydrogen, hydroxy, C₁-C₆ straight alkyl, C₁-C₆ branched alkyl, C₂-C₆ alkenyl, C₃-C₈ cyclic alkyl, heterocyclic, heteroaryl and aryl, wherein all may be optionally substituted by one or more independently selected from the group consisting of halo, alkyl, lower alkyl, acyl, oxo, hydroxy, hydroxyalkyl, heterocyclic, heteroaryl, cyano, amino, aminoalkyl, and carboxy;
R^{14**}and R^{15**}are independently selected from the group consisting of hydrogen, C₁-C₆ straight alkyl, C₁-C₆ branched alkyl, C₂-C₆ alkenyl, C₃-C₈ cyclic alkyl, aryl, heteroaryl, heterocycle, and acyl, wherein all may be optionally substituted by one or more independently selected from the group consisting of halo, alkyl, lower alkyl, alkenyl, cycloalkyl, acyl, hydroxy, hydroxyalkyl, heterocyclic, heteroaryl, aryl, amino, aminoalkyl, oxo, cyano, carboxy, carboxyalkyl, alkoxycarbonyl, and —OR¹⁶;
R^{14**}and R^{15**}taken together may form a 4- to 12-membered monocyclic, bicyclic, tricyclic or benzofused ring;
R^{16**}is independently selected from the group consisting of C₁-C₆ straight alkyl, C₁-C₆ branched alkyl, C₂-C₆ alkenyl, C₃-C₈ cyclic alkyl, heterocyclic, heteroaryl and aryl, wherein all may be substituted by one or more independently selected from the group consisting of halo, alkyl, lower alkyl, acyl, oxo, hydroxy, hydroxyalkyl, heterocyclic, heteroaryl, aryl, amino, aminoalkyl, cyano, and carboxy.

16. The pharmaceutical composition of claim 12, wherein the compound is of Formula IV: 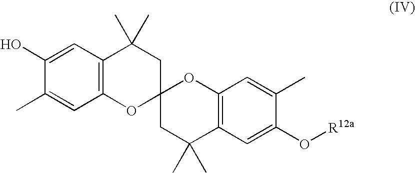
or a pharmaceutically acceptable salt, ester or prodrug thereof, wherein:
R^{12a} is selected from the group consisting of carboxy-C₁-C₆ straight alkyl, carboxy-C₃-C₆ branched alkyl, carboxy-C₂-C₆ alkenyl, carboxy-C₃-C₈ cyclic alkyl, (C(O)NHR^{13a})—C₁-C₆ straight alkyl, (C(O)NHR^{13a})—C₃-C₆ branched alkyl, (C(O)NHR^{13a})—C₂-C₆ alkenyl, (C(O)NHR^{13a})—C₃-C₈ cyclic alkyl, heteroaralkyl, heterocyclicalkyl, and aralkyl, wherein all may be optionally substituted by one or more independently selected from the group consisting of halo, alkyl, lower alkyl, alkenyl, cycloalkyl, acyl, hydroxy, hydroxyalkyl, heterocyclic, heteroaryl, aryl, amino, aminoalkyl, —NR^{14a}R^{15a}, oxo, cyano, carboxy, carboxyalkyl, alkoxycarbonyl, —OR^{16a}, —C(O)R^{16a}, —C(O)—NH₂, —C(O)—N(H)R^{14a}, —C(O)—N(H)OR^{14a}, C(O)—NR^{14a}R^{15a}, NR^{15a}C(O)R^{14a}, —NR^{15a}C(O)NR^{14a}R^{15a}, —OC(O)NR^{14a}R^{15a}, —NR^{15a}C(O)OR^{16a}, —S(O)ₙ—R^{16a}, —S(O)₂—NH₂, —S(O)₂—N(H)R^{14a} and —S(O)₂—NR^{14a}R^{15a}; or
R^{12a} is selected from the group consisting of C₁-C₆ straight alkyl, C₃-C₆ branched alkyl, C₂-C₆ alkenyl, C₃-C₈ cyclic alkyl, hydroxy-C₁-C₆ straight alkyl, hydroxy-C₃-C₆ branched alkyl, hydroxy-C₂-C₆ alkenyl, hydroxy-C₃-C₈ cyclic alkyl, wherein all may be optionally substituted by one or more independently selected from the group consisting of halo, alkyl, lower alkyl, alkenyl, cycloalkyl, acyl, hydroxy, hydroxyalkyl, heterocyclic, heteroaryl, aryl, amino, aminoalkyl, —NR^{14a}R^{15a}, oxo, cyano, carboxy, carboxyalkyl, alkoxycarbonyl, —OR^{16a}, —C(O)R^{16a}, —C(O)—, NH₂, —C(O)—N(H)R^{14a}, —C(O)—N(H)OR^{14a}, —C(O)—NR^{14a}R^{15a}, —NR^{15a}C(O)R^{14a}, —NR^{15a}C(O)NR^{14a}R^{15a}, OC(O)NR^{14a}R^{15a}, —NR^{15a}C(O)OR^{16a}, —S(O)ₙ—R^{16a}, —S(O)₂—NH₂, —S(O)₂—N(H)R^{14a} and —S(O)₂—NR^{14a}R^{15a};
each n is independently 0, 1, or 2;
R^{13a} is selected from the group consisting of hydrogen, hydroxy, C₁-C₆ straight alkyl, C₁-C₆ branched alkyl, C₂-C₆ alkenyl, C₃-C₈ cyclic alkyl, heterocyclic, heteroaryl and aryl, wherein all may be optionally substituted by one or more independently selected from the group consisting of halo, alkyl, lower alkyl, acyl, oxo, hydroxy, hydroxyalkyl, heterocyclic, heteroaryl, cyano, amino, aminoalkyl, and carboxy;
R^{14a} and R^{15a} are independently selected from the group consisting of hydrogen, C₁-C₆ straight alkyl, C₁-C₆ branched alkyl, C₂-C₆ alkenyl, C₃-C₈ cyclic alkyl, aryl, heteroaryl, heterocycle, and acyl, wherein all may be optionally substituted by one or more independently selected from the group consisting of halo, alkyl, lower alkyl, alkenyl, cycloalkyl, acyl, hydroxy, hydroxyalkyl, heterocyclic, heteroaryl, aryl, amino, aminoalkyl, oxo, cyano, carboxy, carboxyalkyl, alkoxycarbonyl, and —OR^{16a};
R^{14a} and R^{15a} taken together may form a 4- to 12-membered monocyclic, bicyclic, tricyclic or benzofused ring;
R^{16a} is selected from the group consisting of C₁-C₆ straight alkyl, C₁-C₆ branched alkyl, C₂-C₆ alkenyl, C₃-C₈ cyclic alkyl, heterocyclic, heteroaryl and aryl, wherein all may be substituted by one or more independently selected from the group consisting of halo, alkyl, lower alkyl, acyl, oxo, hydroxy, hydroxyalkyl, heterocyclic, heteroaryl, aryl, amino, aminoalkyl, cyano, and carboxy.

17. The pharmaceutical composition of claim 12 wherein the compound is in the form of an isolated enantiomer.

18. The pharmaceutical composition of claim 12 wherein R^{12a} includes a chiral center.

19. The pharmaceutical composition of claim 12 wherein the compound is in the form of an isolated diastereomer.

20. The pharmaceutical composition of claim 12 wherein R^{12a} is selected from the group consisting of carboxy-C₁-C₆ straight alkyl, carboxy-C₃-C₆ branched alkyl, carboxy-C₃-C₈ cyclic alkyl, (C(O)NHR^{13a})—C₁-C₆ straight alkyl, (C(O)NHR^{13a})—C₃-C₆ branched alkyl, (C(O)NHR^{13a})—C₃-C₈ cyclic alkyl, heteroaralkyl, and heterocyclicalkyl, wherein all may be optionally substituted by one or more independently selected from the group consisting of halo, lower alkyl, cycloalkyl, acyl, hydroxy, hydroxyalkyl, heterocyclic, heteroaryl, amino, aminoalkyl, —NR^{14a}R^{15a}, oxo, cyano, carboxy, carboxyalkyl, alkoxycarbonyl, —OR^{16a}, —C(O)R^{16a}, —C(O)—NH₂, —C(O)—N(H)R^{14a}, —C(O)—N(H)OR^{14a}, C(O)—NR^{14a}R^{15a} and NR^{15a}C(O)R^{14a}.

21. The pharmaceutical composition of claim 12 wherein R^{12a} is selected from the group consisting of C₁-C₆ straight alkyl, C₃-C₆ branched alkyl, C₂-C₆ alkenyl, C₃-C₈ cyclic alkyl, hydroxy-C₁-C₆ straight alkyl, hydroxy-C₃-C₆ branched alkyl, hydroxy-C₂-C₆ alkenyl, hydroxy-C₃-C₈ cyclic alkyl, wherein all may be optionally substituted by one or more independently selected from the group consisting of halo, lower alkyl, cycloalkyl, acyl, hydroxy, hydroxyalkyl, heterocyclic, heteroaryl, amino, aminoalkyl, —NR^{14a}R^{15a}, oxo, cyano, carboxy, carboxyalkyl, alkoxycarbonyl, —OR^{16a}, —C(O)R^{16a}, —C(O)—NH₂, —C(O)—N(H)R^{14a}, —C(O)—N(H)OR^{14a}, —C(O)—NR^{14a}R^{15a} and NR^{15a}C(O)R^{14a}.

22. The pharmaceutical composition of claim 12 wherein R^{12a} is selected from the group consisting of carboxy-C₁-C₄ straight alkyl and carboxy-C₃-C₆ branched alkyl, wherein all may be optionally substituted by one or more independently selected from the group consisting of halo, lower alkyl, acyl, hydroxy, hydroxyalkyl, heterocyclic, heteroaryl, and amino.

23. The pharmaceutical composition of claim 12 wherein R^{12a} is selected from the group consisting of C₁-C₆ straight alkyl, C₃-C₆ branched alkyl, C₂-C₆ alkenyl, C₃-C₈ cyclic alkyl, hydroxy-C₁-C₆ straight alkyl, hydroxy-C₃-C₆ branched alkyl, hydroxy-C₂-C₆ alkenyl, hydroxy-C₃-C₈ cyclic alkyl, wherein all may be optionally substituted by one or more independently selected from the group consisting of halo, lower alkyl, acyl, hydroxy, hydroxyalkyl, heterocyclic, heteroaryl, amino, aminoalkyl, oxo, cyano, and alkoxycarbonyl.

24. The pharmaceutical composition of claim 12 wherein the compound is 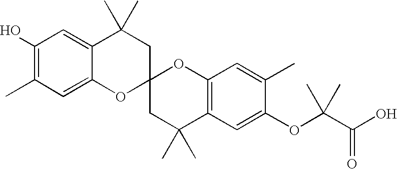
or an enantiomer thereof, or a pharmaceutically acceptable salt, ester or prodrug thereof.

25. The pharmaceutical composition of claim 12 wherein the compound is: 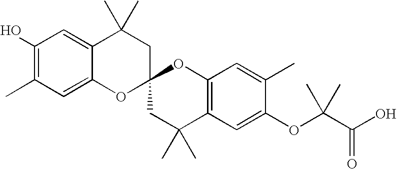
or a pharmaceutically acceptable salt, ester or prodrug thereof.

26. The pharmaceutical composition of claim 12 wherein the compound is 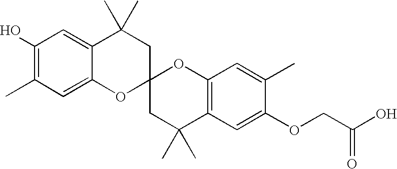
or an enantiomer thereof, or a pharmaceutically acceptable salt, ester or prodrug thereof.

27. A method of treatment or prophylaxis of an inflammatory condition comprising administering a compound of Formula I, optionally in a pharmaceutically acceptable carrier, to a host at risk of, or suffering from, an inflammatory condition 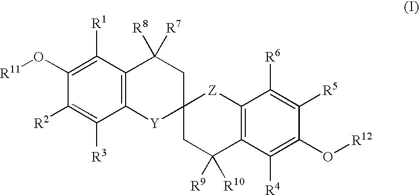
or a pharmaceutically acceptable salt, ester or prodrug thereof, and a pharmaceutically acceptable carrier wherein:
Y and Z are independently O, S(O)_{q}, Se(O)_{q} or N(R¹³);
each q is independently 0, 1 or 2;
R¹, R², R³, R⁴, R⁵ and R⁶ are independently selected from the group consisting of hydrogen, halo, C₁-C₆ straight alkyl, C₁-C₆ branched alkyl, C₂-C₆ alkenyl, C₃-C₈ cyclic alkyl, aryl and —OR⁴, wherein all may be optionally substituted by a hydroxy group;
R⁷, R⁸, R⁹ and R¹⁰ are independently selected from the group consisting of hydrogen, halo, C₁-C₆ straight alkyl, C₁-C₆ branched alkyl, and C₃-C₈ cyclic alkyl, wherein all may be optionally substituted by a hydroxy group;
R¹¹ and R¹² are independently selected from the group consisting of hydrogen, C₁-C₆ straight alkyl, C₁-C₆ branched alkyl, C₂-C₆ alkenyl, C₃-C₈ cyclic alkyl, and acyl, wherein all may be optionally substituted by one or more independently selected from the group consisting of halo, alkyl, lower alkyl, alkenyl, cycloalkyl, acyl, hydroxy, hydroxyalkyl, carboxy, alkoxy, heterocyclic, heteroaryl, aryl, amino, aminoalkyl, —NR¹⁴R¹⁵, oxo, cyano, alkoxycarbonyl, —OR¹⁶, —C(O)R¹⁶, —C(O)—NH₂, —C(O)—N(H)R¹⁴, —C(O)—N(H)OR¹⁴, —C(O)—NR¹⁴R¹⁵, —NR¹⁵C(O)R¹⁴, —NR¹⁵C(O)NR¹⁴R¹⁵, —OC(O)NR¹⁴R¹⁵, —NR¹⁵C(O)OR¹⁶, —S(O)ₙ—R¹⁶, —S(O)₂—NH₂, —S(O)₂—N(H)R¹⁴ and —S(O)₂—NR¹⁴R¹⁵;
or R¹¹ and R¹² are independently selected from the group consisting of hydrogen, aryl, heteroaryl and heterocycle, wherein all may be optionally substituted by one or more independently selected from the group consisting of halo, alkyl, lower alkyl, alkenyl, cycloalkyl, acyl, hydroxy, hydroxyalkyl, alkoxy, heterocyclic, heteroaryl, aryl, amino, aminoalkyl, —NR¹⁴R¹⁵, oxo, cyano, carboxy, carboxyalkyl, alkoxycarbonyl, —OR¹⁶, —C(O)R¹⁶, —C(O)—NH₂, —C(O)—N(H)R¹⁴, —C(O)—N(H)OR¹⁴, —C(O)—NR¹⁴R¹⁵, —NR¹⁵C(O)R¹⁴, —NR¹⁵C(O)NR¹⁴R¹⁵, —OC(O)NR¹⁴R¹⁵, —NR¹⁵C(O)OR¹⁶, —S(O)ₙ—R¹⁶, —S(O)₂—NH₂, —S(O)₂—N(H)R¹⁴ and —S(O)₂—NR¹⁴R¹⁵;
each n is independently 0, 1, or 2;
R¹³ is independently selected from the group consisting of hydrogen, hydroxy, C₁-C₆ straight alkyl, C₁-C₆ branched alkyl, C₂-C₆ alkenyl, C₃-C₈ cyclic alkyl, heterocyclic, heteroaryl and aryl, wherein all may be optionally substituted by one or more independently selected from the group consisting of halo, alkyl, lower alkyl, acyl, oxo, hydroxy, hydroxyalkyl, alkoxy, heterocyclic, heteroaryl, cyano, amino, aminoalkyl, and carboxy;
R¹⁴ and R¹⁵ are independently selected from the group consisting of hydrogen, C₁-C₆ straight alkyl, C₁-C₆ branched alkyl, C₂-C₆ alkenyl, C₃-C₈ cyclic alkyl, aryl, heteroaryl, heterocycle, and acyl, wherein all may be optionally substituted by one or more independently selected from the group consisting of halo, alkyl, lower alkyl, alkenyl, cycloalkyl, acyl, hydroxy, hydroxyalkyl, alkoxy heterocyclic, heteroaryl, aryl, amino, aminoalkyl, oxo, cyano, carboxy, carboxyalkyl, alkoxycarbonyl, and —OR¹⁶;
R¹⁴ and R¹⁵ taken together may form a 4- to 12-membered monocyclic, bicyclic, tricyclic or benzofused ring;
R¹⁶ is independently selected from the group consisting of C₁-C₆ straight alkyl, C₁-C₆ branched alkyl, C₂-C₆ alkenyl, C₃-C₈ cyclic alkyl, heterocyclic, heteroaryl and aryl, wherein all may be substituted by one or more independently selected from the group consisting of halo, alkyl, lower alkyl, acyl, oxo, hydroxy, hydroxyalkyl, heterocyclic, heteroaryl, aryl, amino, aminoalkyl, cyano, and carboxy;
with the proviso, that when R¹¹ and R¹² are heteroaryl, R¹¹ and R¹² cannot be 2-furyl.

28. The method of claim 27 wherein at least one of R¹¹ and R¹² is not hydrogen.

29. The method of claim 27, wherein the compound is of Formula II: 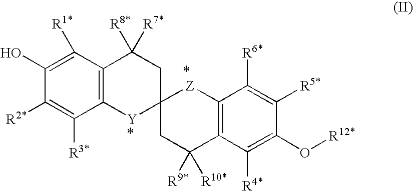
or a pharmaceutically acceptable salt, ester or prodrug thereof, wherein:
Y* and Z* are independently O, S(O)_{q}, Se(O)_{q} or N(R^{13*});
each q is independently 0, 1 or 2;
R^{1*}, R^{2*}, R^{3*}, R^{4*}, R^{5*} and R^{6*} are independently selected from the group consisting of hydrogen, halo, C₁-C₆ straight alkyl, C₁-C₆ branched alkyl, C₂-C₆ alkenyl, C₃-C₈ cyclic alkyl, aryl and —OR¹⁴, wherein all may be optionally substituted by a hydroxy group;
R^{7*}, R^{8*}, R^{9*} and R^{10*} are independently selected from the group consisting of hydrogen, halo, C₁-C₆ straight alkyl, C₁-C₆ branched alkyl, and C₃-C₈ cyclic alkyl, wherein all may be optionally substituted by a hydroxy group;
R^{12*} is selected from the group consisting of C₁-C₆ straight alkyl, hydroxy-C₁-C₆ straight alkyl, polyhydroxy-C₁-C₆ straight alkyl, carboxy-C₁-C₆ straight alkyl, carboxy-C₃-C₆ branched alkyl, carboxy-C₂-C₆ alkenyl, carboxy-C₃-C₈ cyclic alkyl, (C(O)NHR^{13*})—C₁-C₆ straight alkyl, (C(O)NHR^{13*})—C₃-C₆ branched alkyl, (C(O)NHR^{13*})—C₂-C₆ alkenyl, (C(O)NHR^{13*})—C₃-C₈ cyclic alkyl, heteroaralkyl, heterocyclicalkyl, and aralkyl, wherein all may be optionally substituted by one or more independently selected from the group consisting of halo, alkyl, lower alkyl, alkenyl, cycloalkyl, acyl, hydroxy, hydroxyalkyl, heterocyclic, heteroaryl, aryl, amino, aminoalkyl, —NR^{14*}R^{15*}, oxo, cyano, carboxy, carboxyalkyl, alkoxycarbonyl, —OR^{16*}, —C(O)R^{16*}, —C(O)—NH₂, —C(O)—N(H)R^{14*}, C(O)—N(H)OR^{14*}, —C(O)—NR^{14*}R^{15*}, —NR^{15*}, C(O)R^{14*}, —NR^{15*}C(O)NR^{14*}R^{15*}, —OC(O)NR^{14*}R^{15*}, NR^{15*}C(O)OR^{16*}, —S(O)ₙ—R^{16*}, —S(O)₂—NH₂, —S(O)₂—N(H)R^{14*} and —S(O)₂—NR^{14*}R^{15*};
each n is independently 0, 1, or 2;
R^{13*} is selected from the group consisting of hydrogen, hydroxy, C₁-C₆ straight alkyl, C₁-C₆ branched alkyl, C₂-C₆ alkenyl, C₃-C₈ cyclic alkyl, heterocyclic, heteroaryl and aryl, wherein all may be optionally substituted by one or more independently selected from the group consisting of halo, alkyl, lower alkyl, acyl, oxo, hydroxy, hydroxyalkyl, heterocyclic, heteroaryl, cyano, amino, aminoalkyl, and carboxy;
R^{14*} and R^{15*} are independently selected from the group consisting of hydrogen, C₁-C₆ straight alkyl, C₁-C₆ branched alkyl, C₂-C₆ alkenyl, C₃-C₈ cyclic alkyl, aryl, heteroaryl, heterocycle, and acyl, wherein all may be optionally substituted by one or more independently selected from the group consisting of halo, alkyl, lower alkyl, alkenyl, cycloalkyl, acyl, hydroxy, hydroxyalkyl, heterocyclic, heteroaryl, aryl, amino, aminoalkyl, oxo, cyano, carboxy, carboxyalkyl, alkoxycarbonyl, and —OR^{16*};
R^{14*} and R^{15*} taken together may form a 4- to 12-membered monocyclic, bicyclic, tricyclic or benzofused ring;
R^{16*} is independently selected from the group consisting of C₁-C₆ straight alkyl, C₁-C₆ branched alkyl, C₂-C₆ alkenyl, C₃-C₈ cyclic alkyl, heterocyclic, heteroaryl and aryl, wherein all may be substituted by one or more independently selected from the group consisting of halo, alkyl, lower alkyl, acyl, oxo, hydroxy, hydroxyalkyl, heterocyclic, heteroaryl, aryl, amino, aminoalkyl, cyano, and carboxy.

30. The method of claim 27 wherein the compound is of Formula III: 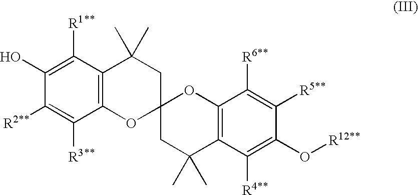
or a pharmaceutically acceptable salt, ester or prodrug thereof wherein:
R^{1**}, R^{2**}, R^{3**}, R^{4**}, R^{5**} and R^{6**}are independently selected from the group consisting of hydrogen, halo, C₁-C₄ straight alkyl, C₁-C₄ branched alkyl, C₂-C₆ alkenyl, aryl and —OR¹⁴;
R^{12**}is selected from the group consisting of C₁-C₆ straight alkyl, hydroxy-C₁-C₆ straight alkyl, polyhydroxy-C₁-C₆ straight alkyl, carboxy-C₁-C₆ straight alkyl, carboxy-C₃-C₆ branched alkyl, carboxy-C₂-C₆ alkenyl, carboxy-C₃-C₈ cyclic alkyl, (C(O)NHR^{3**})—C₁-C₆ straight alkyl, (C(O)NHR^{3**})—C₃-C₆ branched alkyl, (C(O)NHR^{13**})—C₂-C₆ alkenyl, (C(O)NHR^{13**})—C₃-C₈ cyclic alkyl, heteroaralkyl, heterocyclicalkyl, and aralkyl, wherein all may be optionally substituted by one or more independently selected from the group consisting of halo, alkyl, lower alkyl, alkenyl, cycloalkyl, acyl, hydroxy, hydroxyalkyl, heterocyclic, heteroaryl, aryl, amino, aminoalkyl, —NR^{14**}R^{15**}, oxo, cyano, carboxy, carboxyalkyl, alkoxycarbonyl, OR¹⁶, —C(O)R⁶, —C(O)—NH₂, —C(O)—N(H)R^{14**}, —C(O)—N(H)OR^{14**}, —C(O)NR^{14**}R^{15**}, —NR^{15**}C(O)R^{14**}, —NR^{15**}C(O)NR^{14**}R^{15**}, —OC(O)NR^{14**}R^{15**}, —NR^{15**}C(O)OR^{16**}, —S(O)ₙ—R^{16**}, S(O)₂—NH₂, —S(O)₂—N(H)R^{14**}and —S(O)₂—NR^{14**}R^{15**};
each n is independently 0, 1, or 2;
R^{13**} is selected from the group consisting of hydrogen, hydroxy, C₁-C₆ straight alkyl, C₁-C₆ branched alkyl, C₂-C₆ alkenyl, C₃-C₈ cyclic alkyl, heterocyclic, heteroaryl and aryl, wherein all may be optionally substituted by one or more independently selected from the group consisting of halo, alkyl, lower alkyl, acyl, oxo, hydroxy, hydroxyalkyl, heterocyclic, heteroaryl, cyano, amino, aminoalkyl, and carboxy;
R^{14**}and R^{15**} are independently selected from the group consisting of hydrogen, C₁-C₆ straight alkyl, C₁-C₆ branched alkyl, C₂-C₆ alkenyl, C₃-C₈ cyclic alkyl, aryl, heteroaryl, heterocycle, and acyl, wherein all may be optionally substituted by one or more independently selected from the group consisting of halo, alkyl, lower alkyl, alkenyl, cycloalkyl, acyl, hydroxy, hydroxyalkyl, heterocyclic, heteroaryl, aryl, amino, aminoalkyl, oxo, cyano, carboxy, carboxyalkyl, alkoxycarbonyl, and —OR¹⁶;
R^{14**}and R^{15**}taken together may form a 4- to 12-membered monocyclic, bicyclic, tricyclic or benzofused ring;
R^{16**}is independently selected from the group consisting of C₁-C₆ straight alkyl, C₁-C₆ branched alkyl, C₂-C₆ alkenyl, C₃-C₈ cyclic alkyl, heterocyclic, heteroaryl and aryl, wherein all may be substituted by one or more independently selected from the group consisting of halo, alkyl, lower alkyl, acyl, oxo, hydroxy, hydroxyalkyl, heterocyclic, heteroaryl, aryl, amino, aminoalkyl, cyano, and carboxy.

31. The method of claim 27 wherein the compound is of Formula IV: 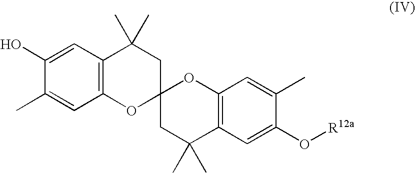
or a pharmaceutically acceptable salt, ester or prodrug thereof, wherein:
R^{12a} is selected from the group consisting of carboxy-C₁-C₆ straight alkyl, carboxy-C₃-C₆ branched alkyl, carboxy-C₂-C₆ alkenyl, carboxy-C₃-C₈ cyclic alkyl, (C(O)NHR^{13a})—C₁-C₆ straight alkyl, (C(O)NHR^{13a})—C₃-C₆ branched alkyl, (C(O)NHR^{13a})—C₂-C₆ alkenyl, (C(O)NHR^{13a})—C₃-C₈ cyclic alkyl, heteroaralkyl, heterocyclicalkyl, and aralkyl, wherein all may be optionally substituted by one or more independently selected from the group consisting of halo, alkyl, lower alkyl, alkenyl, cycloalkyl, acyl, hydroxy, hydroxyalkyl, heterocyclic, heteroaryl, aryl, amino, aminoalkyl, —NR^{14a}R^{15a}, oxo, cyano, carboxy, carboxyalkyl, alkoxycarbonyl, —OR^{16a}, —C(O)R^{16a}, —C(O)—NH₂, —C(O)—N(H)R^{14a}, —C(O)—N(H)OR^{14a}, —C(O)—NR^{14a}R^{15a}NR^{15a}C(O)R^{14a}, —NR^{15a}C(O)NR^{14a}R^{15a}, —OC(O)NR^{14a}R^{15a}, —NR^{15a}C(O)OR^{16a}, —S(O)ₙ—R^{16a}, —S(O)₂—NH₂, —S(O)₂—N(H)R^{14a} and —S(O)₂—NR^{14a}R^{15a}; or
R^{12a} is selected from the group consisting of C₁-C₆ straight alkyl, C₃-C₆ branched alkyl, C₂-C₆ alkenyl, C₃-C₈ cyclic alkyl, hydroxy-C₁-C₆ straight alkyl, hydroxy-C₃-C₆ branched alkyl, hydroxy-C₂-C₆ alkenyl, hydroxy-C₃-C₈ cyclic alkyl, wherein all may be optionally substituted by one or more independently selected from the group consisting of halo, alkyl, lower alkyl, alkenyl, cycloalkyl, acyl, hydroxy, hydroxyalkyl, heterocyclic, heteroaryl, aryl, amino, aminoalkyl, —NR^{14a}R^{15a}, oxo, cyano, carboxy, carboxyalkyl, alkoxycarbonyl, —OR^{16a}, —C(O)R^{16a}, —C(O)—NH₂, —C(O)—N(H)R^{14a}, —C(O)—N(H)OR^{14a}, C(O)—NR^{14a}R^{15a}, —NR^{15a}C(O)R^{14a}, —NR^{15a}C(O)NR^{14a}R^{15a}, —OC(O)NR^{14a}R^{15a}, —NR^{15a}C(O)OR^{16a}, —S(O)ₙ—R^{16a}, —S(O)₂—NH₂, —S(O)₂—N(H)R^{14a} and —S(O)₂—NR^{14a}R^{15a};
each n is independently 0, 1, or 2;
R^{13a} is selected from the group consisting of hydrogen, hydroxy, C₁-C₆ straight alkyl, C₁-C₆ branched alkyl, C₂-C₆ alkenyl, C₃-C₈ cyclic alkyl, heterocyclic, heteroaryl and aryl, wherein all may be optionally substituted by one or more independently selected from the group consisting of halo, alkyl, lower alkyl, acyl, oxo, hydroxy, hydroxyalkyl, heterocyclic, heteroaryl, cyano, amino, aminoalkyl, and carboxy;
R^{14a} and R^{15a} are independently selected from the group consisting of hydrogen, C₁-C₆ straight alkyl, C₁-C₆ branched alkyl, C₂-C₆ alkenyl, C₃-C₈ cyclic alkyl, aryl, heteroaryl, heterocycle, and acyl, wherein all may be optionally substituted by one or more independently selected from the group consisting of halo, alkyl, lower alkyl, alkenyl, cycloalkyl, acyl, hydroxy, hydroxyalkyl, heterocyclic, heteroaryl, aryl, amino, aminoalkyl, oxo, cyano, carboxy, carboxyalkyl, alkoxycarbonyl, and —OR^{16a};
R^{14a} and R^{15a} taken together may form a 4- to 12-membered monocyclic, bicyclic, tricyclic or benzofused ring;
R^{16a} is selected from the group consisting of C₁-C₆ straight alkyl, C₁-C₆ branched alkyl, C₂-C₆ alkenyl, C₃-C₈ cyclic alkyl, heterocyclic, heteroaryl and aryl, wherein all may be substituted by one or more independently selected from the group consisting of halo, alkyl, lower alkyl, acyl, oxo, hydroxy, hydroxyalkyl, heterocyclic, heteroaryl, aryl, amino, aminoalkyl, cyano, and carboxy.

32. The method of claim 27 wherein the compound is in the form of an isolated enantiomer.

33. The method of claim 27 wherein R^{12a} is selected from the group consisting of carboxy-C₁-C₆ straight alkyl, carboxy-C₃-C₆ branched alkyl, carboxy-C₃-C₈ cyclic alkyl, (C(O)NHR^{13a})—C₁-C₆ straight alkyl, (C(O)NHR^{13a})—C₃-C₆ branched alkyl, (C(O)NHR^{13a})—C₃-C₈ cyclic alkyl, heteroaralkyl, and heterocyclicalkyl, wherein all may be optionally substituted by one or more independently selected from the group consisting of halo, lower alkyl, cycloalkyl, acyl, hydroxy, hydroxyalkyl, heterocyclic, heteroaryl, amino, aminoalkyl, —NR^{14a}R^{15a}, oxo, cyano, carboxy, carboxyalkyl, alkoxycarbonyl, —OR^{16a}, —C(O)R^{16a}, —C(O)—NH₂, —C(O)—N(H)R^{14a}, —C(O)—N(H)OR^{14a}, —C(O)—NR^{14a}R^{15a} and —NR^{15a}C(O)R^{14a}.

34. The method of claim 27 wherein R^{12a} is selected from the group consisting of C₁-C₆ straight alkyl, C₃-C₆ branched alkyl, C₂-C₆ alkenyl, C₃-C₈ cyclic alkyl, hydroxy-C₁-C₆ straight alkyl, hydroxy-C₃-C₆ branched alkyl, hydroxy-C₂-C₆ alkenyl, hydroxy-C₃-C₈ cyclic alkyl, wherein all may be optionally substituted by one or more independently selected from the group consisting of halo, lower alkyl, cycloalkyl, acyl, hydroxy, hydroxyalkyl, heterocyclic, heteroaryl, amino, aminoalkyl, —NR^{14a}R^{15a}, oxo, cyano, carboxy, carboxyalkyl, alkoxycarbonyl, —OR^{16a}, —C(O)R^{16a}, —C(O)—NH₂, —C(O)—N(H)R^{14a}, —C(O)—N)OR^{14a}, —C(O)—NR^{14a}R^{15a} and NR^{15a}C(O)R^{14a}.

35. The method of claim 27 wherein the compound is 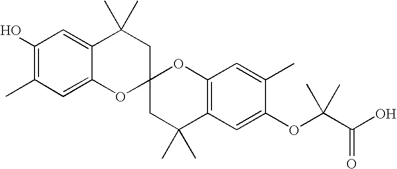
or an enantiomer thereof, or a pharmaceutically acceptable salt, ester or prodrug thereof.

36. The method of claim 27 wherein the compound is: 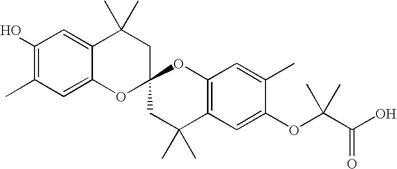
or a pharmaceutically acceptable salt, ester or prodrug thereof.

37. The method of claim 27 wherein the compound is: 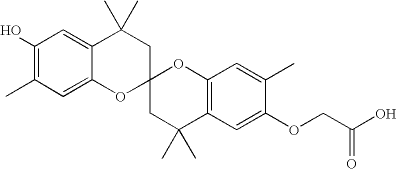
or an enantiomer thereof, or a pharmaceutically acceptable salt, ester or prodrug thereof.

38. The method of claim 27 wherein the inflammatory disorder is a respiratory disorder.

39. The method of claim 27 wherein the inflammatory disorder is asthma or COPD. w
